(19) United States
(12) Patent Application Publication
(10) Pub. No.: US 2023/0174935 A1
(43) Pub. Date: Jun. 8, 2023

(54) REPEAT DOSING OF HYPOIMMUNOGENIC CELLS

(71) Applicant: Sana Biotechnology, Inc., Seattle, WA (US)

(72) Inventor: Sonja SCHREPFER, South San Francisco, CA (US)

(21) Appl. No.: 17/997,103

(22) PCT Filed: Apr. 27, 2021
(86) PCT No.: PCT/US2021/029443

§ 371 Date: Oct. 25, 2022

Related U.S. Application Data
(60) Provisional application No. 63/052,360, filed on Jul. 15, 2020.
(60) Provisional application No. 63/016,190, filed on Apr. 27, 2020.

(51) Int. Cl. C12N 5/0783; C07K 14/705; A61P 37/02

(52) U.S. Cl. CPC C12N 5/0636; A61P 37/02; C07K 14/705; C07K 2319/02; C07K 2319/03; C12N 2506/03

(57) ABSTRACT

Disclosed herein are methods of treating a disorder in a patient by administering immune evading cells. In some embodiments, the patient receives more than one administration of such cells. In some embodiments, the cells disclosed herein have reduced levels or activities of MHC I and/or MHC II human leukocyte antigens. In some embodiments, the cells are derived from primary T cells or pluripotent stem cells that evade immune recognition. In some embodiments, the cells comprise a chimeric antigen receptor.

### CROSS REFERENCE TO RELATED APPLICATIONS

This application claims priority under 35 U.S.C. § 119(e) to U.S. Provisional Application Nos. 63/016,190 filed Apr. 27, 2020 and 63/052,360 filed Jul. 15, 2020, the disclosures of which are herein incorporated by reference in their entireties.

### BACKGROUND

Regenerative cell therapy is an important potential treatment for regenerating injured organs and tissue. With the low availability of organs for transplantation and the accompanying lengthy wait, the possibility of regenerating tissue by transplanting readily available cell lines into patients is understandably appealing. Regenerative cell therapy has shown promising initial results for rehabilitating damaged tissues after transplantation in animal models (e.g., after myocardial infarction). The propensity for the transplant recipient’s immune system to reject allogeneic material, however, greatly reduces the potential efficacy of therapeutics and diminishes the possible positive effects surrounding such treatments.

There is substantial evidence in both animal models and human patients that hypoimmunogenic cell transplantation is a scientifically feasible and clinically promising approach to the treatment of numerous disorders and conditions.

There remains a need for novel approaches, compositions and methods for producing cell-based therapies that avoid detection by the recipient’s immune system.

### SUMMARY

In some embodiments, provided herein is a method for treating a disorder in a patient comprising administering to the patient a therapeutically effective amount of a population of hypoimmunogenic cells comprising exogenous CD47 polypeptides and reduced expression of MHC class I and/or class II human leukocyte antigens, wherein an initial population of such hypoimmunogenic cells had previously been administered to the patient.

In some embodiments, the hypoimmunogenic cells comprise reduced expression of MHC class I and class II human leukocyte antigens. In some embodiments, the hypoimmunogenic cells express the exogenous CD47 polypeptides and reduced expression levels of B2M and/or CIITA. In some embodiments, the hypoimmunogenic cells express the exogenous CD47 polypeptides and reduced expression levels of B2M and CIITA.

In some embodiments, the hypoimmunogenic cells are differentiated cells derived from pluripotent stem cells. In some embodiments, the pluripotent stem cells comprise induced pluripotent stem cells. In some embodiments, the differentiated cells are selected from the group consisting of cardiac cells, neural cells, endothelial cells, pancreatic islet cells, retinal pigmented epithelium cells, hepatocytes, thyroid cells, and T cells.

In some embodiments, the hypoimmunogenic cells comprise cells derived from primary T cells. In certain embodiments, the cells derived from primary T cells are derived from a pool of T cells comprising primary T cells from one or more (e.g., two or more, three or more, four or more, five or more, ten or more, twenty or more, fifty or more, or one hundred or more) subjects different from the patient.

In some embodiments, the cells derived from primary T cells comprise a chimeric antigen receptor.

In some embodiments, the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the antigen binding domain is selected from the group consisting of: (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the antigen binding domain is selected from the group consisting of an antibody, an antigen-binding portion or fragment thereof, an scFv, and a Fab. In some embodiments, the antigen binding domain binds to CD19, CD20, CD22, or BCMA. In some embodiments, the antigen binding domain is an anti-CD19 scFv such as but not limited to FMC63.

In some embodiments, the transmembrane domain comprises one selected from the group consisting of a transmembrane region of TCRα, TCRβ TCRζ,CD3ε CD3y, CD3δ, CD3ζ, CD4,CD5, CD8α, CD8β CD9, CD16, CD28, CD45, CD22, CD33, CD34, CD37, CD40, CD40L/CD154, CD45, CD64, CD80, CD86, OX40/CD134, 4-1BB/CD137, CD154, FcεRIVEGFR2, FAS, FGFR2B, and functional variant thereof.

In some embodiments, the signaling domain(s) comprises a costimulatory domain(s). In certain embodiments, the signaling domain comprises a costimulatory domain. In other embodiments, the signaling domains comprise costimulatory domains. In some cases, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR T cell proliferation, and/or CAR T cell persistence during T cell activation. In some embodiments, the costimulatory domains enhance cytokine production, CAR T cell proliferation, and/or CAR T cell persistence during T cell activation. In some instances, the cytokine gene is an endogenous or exogenous cytokine gene to the hypoimmunogenic cells. In some cases, the cytokine gene encodes a pro-inflammatory cytokine. In some embodiments, the pro-inflammatory cytokine is selected from the group consisting of IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In other embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In certain embodiments, the CAR comprises (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8a hinge and transmembrane domain or functional variant thereof; (iii) a 4-1BB costimulatory domain or functional variant thereof; and (iv) a CD3^ signaling domain or functional variant thereof. In some embodiments, the CAR comprises a (i) an anti-CD19 scFv, an anti-CD20 scFv, an anti-CD22 scFv, or an anti-BCMA scFv; (ii) a CD8a hinge and transmembrane domain or functional variant thereof; (iii) a 4-1BB costimulatory domain or functional variant thereof; and (iv) a CD3ζsignaling domain or functional variant thereof.

In some embodiments, the cells derived from primary T cells comprise reduced expression of an endogenous T cell receptor. In some embodiments, the cells derived from primary T cells comprise reduced expression of cytotoxic T-lymphocyte-associated protein 4 (CTLA4) and/or programmed cell death (PD1).

In some embodiments, the population of the hypoimmunogenic cells is administered at least 3 days or more after the initial administration, optionally at least 4 days, 5 days, 6 days, 7 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 1 month, 2 months, 3 months, 4 months, 5 months, 6 months, 12 months, 18 months, 24 months, 30 months, 36 months, 48 months, 54 months, or 60 months or more. In some embodiments, the population of the hypoimmunogenic cells is administered at least 3 days to at least 7 days or more after the initial administration. In some embodiments, the population of the hypoimmunogenic cells is administered at least 1 month or more after the initial administration. In certain embodiments, the population of the hypoimmunogenic cells is administered at least 2 months or more after the initial administration.

In some embodiments, upon the initial and/or subsequent administrations, the population of hypoimmunogenic cells elicits a reduced level of immune activation or no immune activation in the patient. In some embodiments, upon the initial and/or subsequent administrations, the population of hypoimmunogenic cells elicits a reduced level of systemic TH1 activation or no systemic TH1 activation in the patient. In some embodiments, upon the initial and/or subsequent administrations, the population of hypoimmunogenic cells elicits a reduced level of immune activation of peripheral blood mononuclear cells (PBMCs) or no immune activation of PBMCs in the patient. In some embodiments, upon the initial and/or subsequent administrations, the population of hypoimmunogenic cells elicits a reduced level of donor-specific IgG antibodies or no donor specific IgG antibodies against the hypoimmunogenic cells in the patient. In some embodiments, upon the initial and/or subsequent administrations, the population of hypoimmunogenic cells elicits a reduced level of IgM and IgG antibody production or no IgM and IgG antibody production against the hypoimmunogenic cells in the patient. In some embodiments, upon administration, the population of hypoimmunogenic cells elicits a reduced level of cytotoxic T cell killing or no cytotoxic T cell killing of the hypoimmunogenic cells of the hypoimmunogenic cells in the patient.

In some embodiments, the population of hypoimmunogenic cells of the initial administration are no longer present in the patient at the subsequent administration.

In some embodiments, the patient is not administered an immunosuppressive agent at least 3 days or more before or after the initial administration of the population of hypoimmunogenic cells. In some embodiments, the patient is not administered an immunosuppressive agent at least 3 days or more before or after the initial subsequent administration of the population of hypoimmunogenic cells.

In some embodiments, the hypoimmunogenic cell is selected from the group consisting a pluripotent stem cell, an induced pluripotent stem cell, a T cell differentiated from an induced pluripotent stem cell, a primary T cell, and a cell derived from a primary T cell, and the hypoimmunogenic cell is a B2M^{indel/indel}, CIITA^{Indel/indel} cell comprising the exogenous CD47 polypeptides and optionally a CAR

In some embodiments, provided herein is a method for treating a disorder in a patient comprising administering to the patient therapeutically effective amounts of a population of hypoimmunogenic cells in a dosing regimen comprising a first administration, a recovery period and a second administration, wherein the hypoimmunogenic cells comprise exogenous CD47 polypeptides and reduced expression of MHC class I and/or class II human leukocyte antigens.

In some embodiments, the hypoimmunogenic cells further comprise reduced expression of MHC class I and II human leukocyte antigens. In certain embodiments, the hypoimmunogenic cells express the exogenous CD47 polypeptide and reduced expression levels of B2M and/or CIITA. In other embodiments, the hypoimmunogenic cells express the exogenous CD47 polypeptide and reduced expression levels of B2M and CIITA.

In some embodiments, the hypoimmunogenic cells are differentiated cells derived from pluripotent stem cells. In some embodiments, the pluripotent stem cells comprise induced pluripotent stem cells. In certain embodiments, the differentiated cells are selected from the group consisting of cardiac cells, neural cells, endothelial cells, pancreatic islet cells, retinal pigmented epithelium cells, hepatocytes, thyroid cells, and T cells.

In some embodiments, the hypoimmunogenic cells comprise cells derived from primary T cells.

In some embodiments, the hypoimmunogenic cells comprise cells derived from primary T cells. In certain embodiments, the cells derived from primary T cells are derived from a pool of T cells comprising primary T cells from one or more (e.g., two or more, three or more, four or more, five or more, ten or more, twenty or more, fifty or more, or one hundred or more) subjects different from the patient.

In some embodiments, the cells derived from primary T cells comprise a chimeric antigen receptor.

In some embodiments, the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the antigen binding domain is selected from the group consisting of: (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the antigen binding domain is selected from the group consisting of an antibody, an antigen-binding portion or fragment thereof, an scFv, and a Fab. In some embodiments, the antigen binding domain binds to CD19, CD20, CD22, or BCMA. In some embodiments, the antigen binding domain is an anti-CD19 scFv such as but not limited to FMC63.

In some embodiments, the transmembrane domain comprises one selected from the group consisting of a transmembrane region of TCRα, TCRβ TCRζ,CD3ε CD3y, CD3δ, CD3ζCD4, CD5, CD8α, CD8β CD9, CD16, CD28, CD45, CD22, CD33, CD34, CD37, CD40, CD40L/CD154, CD45, CD64, CD80, CD86, OX40/CD134, 4-1BB/CD137, CD154, FcεRIVEGFR2, FAS, FGFR2B, and functional variant thereof.

In some embodiments, the signaling domain(s) comprises a costimulatory domain(s). In certain embodiments, the signaling domain comprises a costimulatory domain. In other embodiments, the signaling domains comprise costimulatory domains. In some cases, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR T cell proliferation, and/or CAR T cell persistence during T cell activation. In some embodiments, the costimulatory domains enhance cytokine production, CAR T cell proliferation, and/or CAR T cell persistence during T cell activation. In some instances, the cytokine gene is an endogenous or exogenous cytokine gene to the hypoimmunogenic cells. In some cases, the cytokine gene encodes a pro-inflammatory cytokine. In some embodiments, the pro-inflammatory cytokine is selected from the group consisting of IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In other embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In certain embodiments, the CAR comprises (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8a hinge and transmembrane domain or functional variant thereof; (iii) a 4-1BB costimulatory domain or functional variant thereof; and (iv) a CD3ζsignaling domain or functional variant thereof. In some embodiments, the CAR comprises a (i) an anti-CD19 scFv, an anti-CD20 scFv, an anti-CD22 scFv, or an anti-BCMA scFv; (ii) a CD8a hinge and transmembrane domain or functional variant thereof; (iii) a 4-1BB costimulatory domain or functional variant thereof; and (iv) a CD3ζsignaling domain or functional variant thereof.

In some embodiments, the cells derived from primary T cells comprise reduced expression of an endogenous T cell receptor. In some embodiments, the cells derived from primary T cells comprise reduced expression of cytotoxic T-lymphocyte-associated protein 4 (CTLA4) and/or programmed cell death (PD1).

In some embodiments, the recovery period comprises at least 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 1 month, 2 months, 3 months, 4 months, 5 months, 6 months, 12 months, 18 months, 24 months, 30 months, 36 months, 48 months, 54 months, or 60 months or more. In some embodiments, the the recovery period comprises at least 3 days to at least 7 days or more. In some embodiments, the recovery period comprises at least 1 month or more. In some embodiments, the recovery period comprises at least 2 months or more.

In some embodiments, the second administration is initiated when the population of hypoimmunogenic cells from the first administration is no longer detectable in the patient.

In some embodiments, upon the first and/or second administrations, the population of hypoimmunogenic cells elicits a reduced level of immune activation or no immune activation in the patient. In some embodiments, upon the first and/or second administrations, the population of hypoimmunogenic cells elicits a reduced level of systemic TH1 activation or no systemic TH1 activation in the patient. In some embodiments, upon the first and/or second administrations, the population of hypoimmunogenic cells elicits a reduced level of immune activation of peripheral blood mononuclear cells (PBMCs) or no immune activation of PBMCs in the patient. In some embodiments, upon the first and/or second administrations, the population of hypoimmunogenic cells elicits a reduced level of donor-specific IgG antibodies or no donor-specific IgG antibodies against the hypoimmunogenic cells in the patient. In some embodiments, upon the first and/or second administrations, the population of hypoimmunogenic cells elicits a reduced level of IgM and IgG antibody production or no IgM and IgG antibody production against the hypoimmunogenic cells in the patient. In some embodiments, upon the first and/or second administrations, the population of hypoimmunogenic cells elicits a reduced level of cytotoxic T cell killing or no cytotoxic T cell killing of the hypoimmunogenic cells in the patient.

In some embodiments, the patient is not administered an immunosuppressive agent at least 3 days or more before or after the first administration of the population of hypoimmunogenic cells. In some embodiments, the patient is not administered an immunosuppressive agent at least 3 days or more before or after the second administration of the population of hypoimmunogenic cells. In some embodiments, the patient is not administered an immunosuppressive agent during the recovery period.

In some embodiments, the hypoimmunogenic cell is selected from the group consisting a pluripotent stem cell, an induced pluripotent stem cell, a T cell differentiated from an induced pluripotent stem cell, a primary T cell, and a cell derived from a primary T cell, and the hypoimmunogenic cell is a B2M^{indel/indel}, CIITA ^{indel/indel} cell comprising the exogenous CD47 polypeptides and optionally a CAR.

In some embodiments, provided herein is method for treating a disorder in a patient by administering cells that do not trigger a systemic acute cellular immune response in the patient, the method comprising: (a) administering a therapeutically effective amount of a first population of cells to the patient; and (b) administering a therapeutically effective amount of a second population of cells to the patient following a recovery period after step (a), wherein the cells of the first and second populations of cells comprise exogenous CD47 polypeptides and reduced expression of MHC class I and/or II human leukocyte antigens, and wherein the recovery period comprises at least 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 1 month, 2 months, 3 months, 4 months, 5 months, 6 months, 12 months, 18 months, 24 months, 30 months, 36 months, 48 months, 54 months, or 60 months or more.

In some embodiments, the cells of the first and second populations comprise reduced expression of MHC class I and MHC class II human leukocyte antigens. In some embodiments, the cells of the first and second populations comprise the exogenous CD47 polypeptides and reduced expression levels of B2M and/or CIITA. In many embodiments, the cells of the first and second populations comprise the exogenous CD47 polypeptides and reduced expression levels of B2M and CIITA.

In some embodiments, the first and second populations of cells comprise differentiated cells derived from pluripotent stem cells. In many embodiments, the pluripotent stem cells comprise induced pluripotent stem cells. In one embodiment, the differentiated cells are selected from the group consisting of cardiac cells, neural cells, endothelial cells, pancreatic islet cells, retinal pigmented epithelium cells, hepatocytes, thyroid cells, and T cells.

In some embodiments, the first and second populations of cells comprises cells derived from primary T cells. In certain embodiments, the cells derived from primary T cells are derived from a pool of T cells comprising primary T cells from one or more (e.g., two or more, three or more, four or more, five or more, ten or more, twenty or more, fifty or more, or one hundred or more) subjects different from the patient.

In some embodiments, the cells derived from primary T cells comprise a chimeric antigen receptor.

In some embodiments, the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the antigen binding domain is selected from the group consisting of: (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the antigen binding domain is selected from the group consisting of an antibody, an antigen-binding portion or fragment thereof, an scFv, and a Fab. In some embodiments, the antigen binding domain binds to CD19, CD20, CD22, or BCMA. In some embodiments, the antigen binding domain is an anti-CD19 scFv such as but not limited to FMC63.

In some embodiments, the transmembrane domain comprises one selected from the group consisting of a transmembrane region of TCRα, TCRβ TCRζ,CD3ε CD3y, CD3δ, CD3ζCD4, CD5, CD8α, CD8β CD9, CD16, CD28, CD45, CD22, CD33, CD34, CD37, CD40, CD40L/CD154, CD45, CD64, CD80, CD86, OX40/CD134, 4-1BB/CD137, CD154, FcεRIVEGFR2, FAS, FGFR2B, and functional variant thereof.

In some embodiments, the signaling domain(s) comprises a costimulatory domain(s). In certain embodiments, the signaling domain comprises a costimulatory domain. In other embodiments, the signaling domains comprise costimulatory domains. In some cases, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR T cell proliferation, and/or CAR T cell persistence during T cell activation. In some embodiments, the costimulatory domains enhance cytokine production, CAR T cell proliferation, and/or CAR T cell persistence during T cell activation. In some instances, the cytokine gene is an endogenous or exogenous cytokine gene to the hypoimmunogenic cells. In some cases, the cytokine gene encodes a pro-inflammatory cytokine. In some embodiments, the pro-inflammatory cytokine is selected from the group consisting of IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In other embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In certain embodiments, the CAR comprises (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8a hinge and transmembrane domain or functional variant thereof; (iii) a 4-1BB costimulatory domain or functional variant thereof; and (iv) a CD3ζsignaling domain or functional variant thereof. In some embodiments, the CAR comprises a (i) an anti-CD19 scFv, an anti-CD20 scFv, an anti-CD22 scFv, or an anti-BCMA scFv; (ii) a CD8a hinge and transmembrane domain or functional variant thereof; (iii) a 4-1BB costimulatory domain or functional variant thereof; and (iv) a CD3ζsignaling domain or functional variant thereof.

In some embodiments, the cells derived from primary T cells comprise reduced expression of an endogenous T cell receptor. In some embodiments, the cells derived from primary T cells comprise reduced expression of cytotoxic T-lymphocyte-associated protein 4 (CTLA4) and/or programmed cell death (PD1).

In some embodiments, the recovery period comprises at least 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 1 month, 2 months, 3 months, 4 months, 5 months, 6 months, 12 months, 18 months, 24 months, 30 months, 36 months, 48 months, 54 months, or 60 months or more. In some embodiments, the recovery period comprises at least 2 months or more. In some embodiments, the step (b) is performed when the first population of cells is no longer detectable in the patient.

In some embodiments, the first and/or second population of cells elicits a reduced level of immune activation or no immune activation in the patient. In some embodiments, the first and/or second population of cells elicits a reduced level of systemic TH1 activation or no systemic TH1 activation in the patient. In some embodiments, the first and/or second population of cells elicits a reduced level of immune activation of peripheral blood mononuclear cells (PBMCs) or no immune activation of PBMCs in the patient. In some embodiments, the first and/or second population of cells elicits a reduced level of donor-specific IgG antibodies or no donor-specific IgG antibodies against the administered cells in the patient. In some embodiments, the first and/or second population of cells elicits a reduced level of IgM and IgG antibody production or no IgM and IgG antibody production against the c administered ells in the patient. In some embodiments, the first and/or second population of cells elicits a reduced level of cytotoxic T cell killing or no cytotoxic T cell killing of the administered cells in the patient.

In some embodiments, the patient is not administered an immunosuppressive agent at least 3 days or more before or after the administration of the first population of cells. In some embodiments, the patient is not administered an immunosuppressive agent at least 3 days or more before or after the administration of the second population of cells. In some embodiments, the patient is not administered an immunosuppressive agent during the recovery period.

In some embodiments, the hypoimmunogenic cell is selected from the group consisting a pluripotent stem cell, an induced pluripotent stem cell, a T cell differentiated from an induced pluripotent stem cell, a primary T cell, and a cell derived from a primary T cell, and the hypoimmunogenic cell is a B2M^{indel/indel}, CIITA^{indel/indel} cell comprising the exogenous CD47 polypeptides and optionally a CAR.

In some embodiments, the present disclosure describes use of a population of hypoimmunogenic cells comprising exogenous CD47 polypeptides and reduced expression of MHC class I and/or class II human leukocyte antigens for treatment of a disorder in a patient, wherein the patient has previously received an initial population of such hypoimmunogenic cells.

In some embodiments, the present disclosure describes use of a population of hypoimmunogenic cells comprising exogenous CD47 polypeptides and reduced expression of MHC class I and class II human leukocyte antigens for treatment of a disorder in a patient, wherein the patient has previously received an initial population of such hypoimmunogenic cells.

In some embodiments, the present disclosure describes use of a population of hypoimmunogenic cells comprising exogenous CD47 polypeptides and reduced levels of B2M and CIITA polypeptides for treatment of a disorder in a patient, wherein the patient has previously received an initial population of such hypoimmunogenic cells.

In some embodiments, the present disclosure describes use of a population of hypoimmunogenic cells comprising exogenous CD47 polypeptides, a genomic modification of the B2M gene, and a genomic modification of the CIITA gene for treatment of a disorder in a patient, wherein the patient has previously received an initial population of such hypoimmunogenic cells.

In some embodiments, the population of hypoimmunogenic cells comprises differentiated cells derived from pluripotent stem cells.

In some embodiments, the pluripotent stem cells comprise induced pluripotent stem cells.

In some embodiments, the differentiated cells are selected from the group consisting of cardiac cells, neural cells, endothelial cells, pancreatic islet cells, retinal pigmented epithelium cells, hepatocytes, thyroid cells, and T cells.

In some embodiments, the population of hypoimmunogenic cells comprises cells derived from primary T cells. In certain embodiments, the cells derived from primary T cells are derived from a pool of T cells comprising primary T cells from one or more (e.g., two or more, three or more, four or more, five or more, ten or more, twenty or more, fifty or more, or one hundred or more) subjects different from the patient.

In some embodiments, the cells derived from primary T cells comprise a chimeric antigen receptor.

In some embodiments, the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the antigen binding domain is selected from the group consisting of: (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the antigen binding domain is selected from the group consisting of an antibody, an antigen-binding portion or fragment thereof, an scFv, and a Fab. In some embodiments, the antigen binding domain binds to CD19, CD20, CD22, or BCMA. In some embodiments, the antigen binding domain is an anti-CD19 scFv such as but not limited to FMC63.

In some embodiments, the transmembrane domain comprises one selected from the group consisting of a transmembrane region of TCRα, TCRβ,TCRζ,CD3ε CD3y, CD3δ, CD3ζCD4, CD5, CD8α, CD8β CD9, CD16, CD28, CD45, CD22, CD33, CD34, CD37, CD40, CD40L/CD154, CD45, CD64, CD80, CD86, OX40/CD134, 4-1BB/CD137, CD154, FcεRIVEGFR2, FAS, FGFR2B, and functional variant thereof.

In some embodiments, the signaling domain(s) comprises a costimulatory domain(s). In certain embodiments, the signaling domain comprises a costimulatory domain. In other embodiments, the signaling domains comprise costimulatory domains. In some cases, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR T cell proliferation, and/or CAR T cell persistence during T cell activation. In some embodiments, the costimulatory domains enhance cytokine production, CAR T cell proliferation, and/or CAR T cell persistence during T cell activation. In some instances, the cytokine gene is an endogenous or exogenous cytokine gene to the hypoimmunogenic cells. In some cases, the cytokine gene encodes a pro-inflammatory cytokine. In some embodiments, the pro-inflammatory cytokine is selected from the group consisting of IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In other embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In certain embodiments, the CAR comprises (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8a hinge and transmembrane domain or functional variant thereof; (iii) a 4-1BB costimulatory domain or functional variant thereof; and (iv) a CD3ζsignaling domain or functional variant thereof. In some embodiments, the CAR comprises a (i) an anti-CD19 scFv, an anti-CD20 scFv, an anti-CD22 scFv, or an anti-BCMA scFv; (ii) a CD8a hinge and transmembrane domain or functional variant thereof; (iii) a 4-1BB costimulatory domain or functional variant thereof; and (iv) a CD3ζsignaling domain or functional variant thereof.

In some embodiments, the cells derived from primary T cells comprise reduced expression of an endogenous T cell receptor. In some embodiments, the cells derived from primary T cells comprise reduced expression of cytotoxic T-lymphocyte-associated protein 4 (CTLA4) and/or programmed cell death (PD1).

In some embodiments, the present disclosure provides the use of the population of hypoimmunogenic T cells as described herein, wherein the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the present disclosure provides the use of the population of hypoimmunogenic T cells as described herein, wherein the antigen binding domain is selected from the group consisting of: (a) an antigen binding domain targets an antigen characteristic of a neoplastic cell; (b) an antigen binding domain that targets an antigen characteristic of a T cell, (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the present disclosure provides the use of the population of hypoimmunogenic T cells as described herein, wherein the antigen binding domain is selected from the group consisting of an antibody, an antigen-binding portion thereof, an scFv, and a Fab.

In some embodiments, the present disclosure provides the use of the population of hypoimmunogenic T cells as described herein, wherein the antigen binding domain binds to CD19, CD20, CD22, or BCMA.

In some embodiments, the present disclosure provides the use of the population of hypoimmunogenic T cells as described herein, wherein the transmembrane domain comprises one selected from the group consisting of a transmembrane region of TCRα, TCRβ,TCRζ, CD3ε CD3y, CD3δ,CD3ζCD4, CD5, CD8α, CD8β CD9, CD16, CD28, CD45, CD22, CD33, CD34, CD37, CD40, CD40L/CD154, CD45, CD64, CD80, CD86, OX40/CD134, 4-1BB/CD137, CD154, FcεRIVEGFR2, FAS, FGFR2B, and functional variant thereof.

In some embodiments, the present disclosure provides the use of the population of hypoimmunogenic T cells as described herein, wherein the signaling domain(s) comprises a costimulatory domain(s). In some embodiments, the present disclosure provides the use of the population of hypoimmunogenic T cells as described herein, wherein the costimulatory domains comprise two costimulatory domains that are not the same. In some embodiments, the present disclosure provides the use of the population of hypoimmunogenic cells as described herein, wherein the costimulatory domain(s) enhances cytokine production, CAR T cell proliferation, and/or CAR T cell persistence during T cell activation. In some embodiments, the present disclosure provides the use of the population of hypoimmunogenic cells as described herein, wherein the cytokine gene is an endogenous or exogenous cytokine gene to the hypoimmunogenic cells. In some embodiments, the present disclosure provides the use of the population of hypoimmunogenic T cells as described herein, wherein the cytokine gene encodes a pro-inflammatory cytokine. In some embodiments, the present disclosure provides the use of the population of hypoimmunogenic T cells as described herein, wherein the pro-inflammatory cytokine is selected from the group consisting of IL-1, IL-2, IL-9, IL-12, IL-18, TNF, IFN-gamma, and a functional fragment thereof.

In some embodiments, the present disclosure provides the use of the population of hypoimmunogenic T cells as described herein, wherein the domain which upon successful signaling of the CAR induces expression of the cytokine gene comprises a transcription factor or functional domain or fragment thereof. In some embodiments, the present disclosure provides the use of the population of hypoimmunogenic T cells as described herein, wherein the CAR comprises a CD3 zeta domain or an immunoreceptor tyrosine-based activation motif (ITAM), or functional variant thereof. In some embodiments, the present disclosure provides the use of the population of hypoimmunogenic T cells as described herein, wherein the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the present disclosure provides the use of the population of hypoimmunogenic T cells as described herein, wherein the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the present disclosure provides the use of the population of hypoimmunogenic T cells as described herein, wherein the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the present disclosure provides the use of the population of hypoimmunogenic T cells as described herein, wherein the CAR comprises a (i) an anti-CD19 scFv; (ii) a CD8a hinge and transmembrane domain or functional variant thereof; (iii) a 4-1BB costimulatory domain or functional variant thereof; and (iv) a CD3ζsignaling domain.

In some embodiments, the present disclosure provides the use of the population of hypoimmunogenic cells, wherein the hypoimmunogenic cell is selected from the group consisting a pluripotent stem cell, an induced pluripotent stem cell, a T cell differentiated from an induced pluripotent stem cell, a primary T cell, and a cell derived from a primary T cell, and the hypoimmunogenic cell comprises exogenous CD47 and/or optionally a CAR, and wherein the hypoimmunogenic cell is optionally a B2M^{indel/indel} cell and/or optionally CIITA^{indel/indel} cell.

In some embodiments, provided herein is a population of hypoimmunogenic cells comprising exogenous CD47 polypeptides and reduced expression of MHC class I or class II human leukocyte antigens, wherein the population of hypoimmunogenic cells comprises cells derived from primary T cells. In some embodiments, provided herein is a population of hypoimmunogenic cells comprising exogenous CD47 polypeptides and reduced expression of MHC class I and class II human leukocyte antigens, wherein the population of hypoimmunogenic cells comprises cells derived from primary T cells.

In some embodiments, provided herein is a population of hypoimmunogenic cells comprising exogenous CD47 polypeptides and reduced levels of B2M and CIITA polypeptides, wherein the population of hypoimmunogenic cells comprises cells derived from primary T cells.

In some embodiments, provided herein is a population of hypoimmunogenic cells comprising exogenous CD47 polypeptides, a genomic modification of the B2M gene, and a genomic modification of the CIITA gene, wherein the population of hypoimmunogenic cells comprises cells derived from primary T cells.

In some embodiments, the population of hypoimmunogenic cells comprises differentiated cells derived from pluripotent stem cells. In some embodiments, the pluripotent stem cells comprise induced pluripotent stem cells. In some embodiments, the differentiated cells are selected from the group consisting of cardiac cells, neural cells, endothelial cells, pancreatic islet cells, retinal pigmented epithelium cells, hepatocytes, thyroid cells, and T cells.

In some embodiments, the population of hypoimmunogenic cells comprises cells derived from primary T cells. In certain embodiments, the cells derived from primary T cells are derived from a pool of T cells comprising primary T cells from one or more (e.g., two or more, three or more, four or more, five or more, ten or more, twenty or more, fifty or more, or one hundred or more) subjects different from the patient.

In some embodiments, the cells derived from primary T cells comprise a chimeric antigen receptor.

In some embodiments, the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transme+mbrane domain, three or four signaling domains, and a domain which upon successful signaling of the CAR induces expression of a cytokine gene.

In some embodiments, the antigen binding domain is selected from the group consisting of: (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the antigen binding domain is selected from the group consisting of an antibody, an antigen-binding portion or fragment thereof, an scFv, and a Fab. In some embodiments, the antigen binding domain binds to CD19, CD20, CD22, or BCMA. In some embodiments, the antigen binding domain is an anti-CD19 scFv such as but not limited to FMC63.

In some embodiments, the transmembrane domain comprises one selected from the group consisting of a transmembrane region of TCRα, TCRβ,TCRζ,CD3ε CD3y, CD3δ, CD3ζCD4, CD5, CD8α, CD8β CD9, CD16, CD28, CD45, CD22, CD33, CD34, CD37, CD40, CD40L/CD154, CD45, CD64, CD80, CD86, OX40/CD134, 4-1BB/CD137, CD154, FcεRIVEGFR2, FAS, FGFR2B, and functional variant thereof.

In some embodiments, the signaling domain(s) comprises a costimulatory domain(s). In certain embodiments, the signaling domain comprises a costimulatory domain. In other embodiments, the signaling domains comprise costimulatory domains. In some cases, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR T cell proliferation, and/or CAR T cell persistence during T cell activation. In some embodiments, the costimulatory domains enhance cytokine production, CAR T cell proliferation, and/or CAR T cell persistence during T cell activation. In some instances, the cytokine gene is an endogenous or exogenous cytokine gene to the hypoimmunogenic cells. In some cases, the cytokine gene encodes a pro-inflammatory cytokine. In some embodiments, the pro-inflammatory cytokine is selected from the group consisting of IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In other embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In certain embodiments, the CAR comprises (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8a hinge and transmembrane domain or functional variant thereof; (iii) a 4-1BB costimulatory domain or functional variant thereof; and (iv) a CD3ζsignaling domain or functional variant thereof. In some embodiments, the CAR comprises a (i) an anti-CD19 scFv, an anti-CD20 scFv, an anti-CD22 scFv, or an anti-BCMA scFv; (ii) a CD8a hinge and transmembrane domain or functional variant thereof; (iii) a 4-1BB costimulatory domain or functional variant thereof; and (iv) a CD3ζsignaling domain or functional variant thereof.

In some embodiments, the cells derived from primary T cells comprise reduced expression of an endogenous T cell receptor. In some embodiments, the cells derived from primary T cells comprise reduced expression of cytotoxic T-lymphocyte-associated protein 4 (CTLA4) and/or programmed cell death (PD1).

In some embodiments, the present disclosure provides the use of a population of hypoimmunogenic cells comprising exogenous CD47 polypeptides and reduced expression of MHC class I and/or class II human leukocyte antigens, wherein the population of hypoimmunogenic cells comprises cells derived from primary T cells.

In some embodiments, the present disclosure provides the use of a population of hypoimmunogenic cells comprising exogenous CD47 polypeptides and reduced expression of MHC class I and class II human leukocyte antigens, wherein the population of hypoimmunogenic cells comprises cells derived from primary T cells.

In some embodiments, the present disclosure provides the use of a population of hypoimmunogenic cells comprising exogenous CD47 polypeptides and reduced levels of B2M and CIITA polypeptides, wherein the population of hypoimmunogenic cells comprises cells derived from primary T cells.

In some embodiments, the present disclosure provides the use of a population of hypoimmunogenic cells comprising exogenous CD47 polypeptides, a genomic modification of the B2M gene, and a genomic modification of the CIITA gene, wherein the population of hypoimmunogenic cells comprises cells derived from primary T cells.

In some embodiments, the population of hypoimmunogenic cells comprise any such cells disclosed herein. In some embodiments, the population of hypoimmunogenic cells are for use in treatment of a disorder in a patient, wherein the patient has previously received an initial population of such hypoimmunogenic cells.

In some embodiments of the population of hypoimmunogenic cells, the hypoimmunogenic cell or cells is selected from the group consisting a pluripotent stem cell, an induced pluripotent stem cell, a T cell differentiated from an induced pluripotent stem cell, a primary T cell, and a cell derived from a primary T cell, and the hypoimmunogenic cell comprises exogenous CD47 and/or optionally a CAR, and wherein the cell or cells is optionally a B2M^{indel/indel} cell and/or optionally CIITA^{indel/indel} cell.

In some embodiments, the present disclosure provides the use of a population of hypoimmunogenic cells comprising exogenous CD47 polypeptides and reduced expression of MHC class I and/or class II human leukocyte antigens, wherein the population of hypoimmunogenic cells comprises cells derived from primary T cells.

In some embodiments, the present disclosure provides the use of a population of hypoimmunogenic cells comprising exogenous CD47 polypeptides and reduced expression of MHC class I and class II human leukocyte antigens, wherein the population of hypoimmunogenic cells comprises cells derived from primary T cells.

In some embodiments, the present disclosure provides the use of a population of hypoimmunogenic cells comprising exogenous CD47 polypeptides and reduced levels of B2M and CIITA polypeptides, wherein the population of hypoimmunogenic cells comprises cells derived from primary T cells.

In some embodiments, the present disclosure provides the use of a population of hypoimmunogenic cells comprising exogenous CD47 polypeptides, a genomic modification of the B2M gene, and a genomic modification of the CIITA gene, wherein the population of hypoimmunogenic cells comprises cells derived from primary T cells.

In some embodiments, the present disclosure provides the use of the population of hypoimmunogenic cells described herein, wherein the cells derived from primary T cells are derived from a pool of T cells comprising T cells from one or more subjects different from the patient.

In some embodiments, the present disclosure provides the use of the population of hypoimmunogenic cells described herein, wherein the cells derived from primary T cells comprise a chimeric antigen receptor (CAR).

In some embodiments, the present disclosure provides the use of the population of hypoimmunogenic cells described herein, wherein the cells derived from primary T cells comprise reduced expression of an endogenous T cell receptor.

In some embodiments, the present disclosure provides the use of the population of hypoimmunogenic cells described herein, wherein the cells derived from primary T cells comprise reduced expression of cytotoxic T-lymphocyte-associated protein 4 (CTLA4) and/or programmed cell death (PD1).

In some embodiments, the present disclosure provides the use of the population of hypoimmunogenic T cells described herein, wherein the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the present disclosure provides the use of the population of hypoimmunogenic T cells described herein, wherein the antigen binding domain is selected from the group consisting of: (a) an antigen binding domain targets an antigen characteristic of a neoplastic cell; (b) an antigen binding domain that targets an antigen characteristic of a T cell, (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the present disclosure provides the use of the population of hypoimmunogenic T cells described herein, wherein the antigen binding domain is selected from the group consisting of an antibody, an antigen-binding portion thereof, an scFv, and a Fab.

In some embodiments, the present disclosure provides the use of the population of hypoimmunogenic T cells described herein, wherein the antigen binding domain binds to CD19, CD20, CD22, or BCMA.

In some embodiments, the present disclosure provides the use of the population of hypoimmunogenic T cells described herein, wherein the transmembrane domain comprises one selected from the group consisting of a transmembrane region of TCRα, TCRβ TCRζ, CD3ε CD3y, CD3δ,CD3ζ,CD4, CD5, CD8α, CD8β CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

In some embodiments, the present disclosure provides the use of the population of hypoimmunogenic T cells described herein, wherein the signaling domain(s) comprises a costimulatory domain(s). In some embodiments, the present disclosure provides the use of the population of hypoimmunogenic cells described herein, wherein the costimulatory domains comprise two costimulatory domains that are not the same. In some embodiments, the present disclosure provides the use of the population of hypoimmunogenic cells described herein, wherein the costimulatory domain(s) enhances cytokine production, CAR T cell proliferation, and/or CAR T cell persistence during T cell activation. In some embodiments, the present disclosure provides the use of the population of hypoimmunogenic cells described herein, wherein the cytokine gene is an endogenous or exogenous cytokine gene to the hypoimmunogenic cells. In some embodiments, the present disclosure provides the use of the population of hypoimmunogenic cells described herein, wherein the cytokine gene encodes a pro-inflammatory cytokine.

In some embodiments, the present disclosure provides the use of the population of hypoimmunogenic T cells described herein, wherein the pro-inflammatory cytokine is selected from the group consisting of IL-1, IL-2, IL-9, IL-12, IL-18, TNF, IFN-gamma, and a functional fragment thereof.

In some embodiments, the present disclosure provides the use of the population of hypoimmunogenic T cells described herein, wherein the domain which upon successful signaling of the CAR induces expression of the cytokine gene comprises a transcription factor or functional domain or fragment thereof. In some embodiments, the present disclosure provides the use of the population of hypoimmunogenic cells described herein, wherein the CAR comprises a CD3 zeta domain or an immunoreceptor tyrosine-based activation motif (ITAM), or functional variant thereof. In some embodiments, the present disclosure provides the use of the population of hypoimmunogenic cells described herein, wherein the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the present disclosure provides the use of the population of hypoimmunogenic cells described herein, wherein the CAR comprises a (i) a CD3 zeta domain, or an immunoreceptor tyrosine-based activation motif (ITAM), or functional variant thereof; (ii) a CD28 domain or functional variant thereof; and (iii) a 4-1BB costimulatory domain, or a CD134 domain, or functional variant thereof. In some embodiments, the present disclosure provides the use of the population of hypoimmunogenic cells described herein, wherein the CAR comprises a (i) a CD3 zeta domain, or an immunoreceptor tyrosine-based activation motif (ITAM), or functional variant thereof; (ii) a CD28 domain or functional variant thereof; (iii) a 4-1BB costimulatory domain, or a CD134 domain, or functional variant thereof; and (iv) a cytokine or costimulatory ligand transgene. In some embodiments, the present disclosure provides the use of the population of hypoimmunogenic cells described herein, wherein the CAR comprises a (i) an anti-CD19 scFv; (ii) a CD8a hinge and transmembrane domain or functional variant thereof; (iii) a 4-1BB costimulatory domain or functional variant thereof; and (iv) a CD3ζsignaling domain. In some embodiments, the CAR comprises a (i) an anti-CD19 scFv, an anti-CD20 scFv, an anti-CD22 scFv, or an anti-BCMA scFv; (ii) a CD8a hinge and transmembrane domain or functional variant thereof; (iii) a 4-1BB costimulatory domain or functional variant thereof; and (iv) a CD3ζsignaling domain or functional variant thereof.

In some embodiments, the present disclosure provides the use of the population of hypoimmunogenic cells described herein, for use in treatment of a disorder in a patient, wherein the patient has previously received an initial population of such hypoimmunogenic cells.

In some embodiments, the present disclosure provides the use of the population of hypoimmunogenic cells described herein, wherein the hypoimmunogenic cell is selected from the group consisting a pluripotent stem cell, an induced pluripotent stem cell, a T cell differentiated from an induced pluripotent stem cell, a primary T cell, and a cell derived from a primary T cell, and the hypoimmunogenic cell comprises exogenous CD47 and/or optionally a CAR, and wherein the hypoimmunogenic cell is optionally a B2M^{indel/indel} cell and/or optionally CIIT A^{indel/indel} cell.

In some embodiments, the present disclosure provides a method for treating a disorder in a patient comprising administering to the patient a therapeutically effective amount of a population of hypoimmunogenic T cells derived from primary T cells and which comprise exogenous CD47 polypeptides and exhibit reduced expression of MHC class I and/or class II human leukocyte antigens, wherein an initial population of such hypoimmunogenic T cells had previously been administered to the patient. In some embodiments, the present disclosure provides a method for treating a disorder in a patient comprising administering to the patient a therapeutically effective amount of a population of hypoimmunogenic T cells derived from primary T cells and which comprise exogenous CD47 polypeptides and exhibit reduced expression of MHC class I and/or class II human leukocyte antigens, wherein an initial population of such hypoimmunogenic T cells or an initial population of hypoimmunogenic cells differentiated from hypoimmune induced pluripotent stem cells had previously been administered to the patient.

In some embodiments, the hypoimmunogenic T cells comprise reduced expression of MHC class I and class II human leukocyte antigens.

In some embodiments, the hypoimmunogenic T cells express the exogenous CD47 polypeptides and reduced expression levels of B2M and/or CIITA.

In some embodiments, the hypoimmunogenic T cells express the exogenous CD47 polypeptides and reduced expression levels of B2M and CIITA.

In some embodiments, the hypoimmunogenic T cells comprise a chimeric antigen receptor.

In some embodiments, the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the antigen binding domain is selected from the group consisting of: (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the antigen binding domain is selected from the group consisting of an antibody, an antigen-binding portion thereof, an scFv, and a Fab.

In some embodiments, the antigen binding domain binds to CD19, CD20, CD22, or BCMA.

In some embodiments, the transmembrane domain comprises one selected from the group consisting of a transmembrane region of TCRα, TCRβ,TCRζ,CD3ε CD3y, CD3δ, CD3ζCD4, CD5, CD8α, CD8β CD9, CD16, CD28, CD45, CD22, CD33, CD34, CD37, CD40, CD40L/CD154, CD45, CD64, CD80, CD86, OX40/CD134, 4-1BB/CD137, CD154, FcεRIVEGFR2, FAS, FGFR2B, and functional variant thereof.

In some embodiments, the signaling domain(s) comprises a costimulatory domain(s).

In some embodiments, the costimulatory domains comprise two costimulatory domains that are not the same.

In some embodiments, the costimulatory domain(s) enhances cytokine production, CAR T cell proliferation, and/or CAR T cell persistence during T cell activation.

In some embodiments, the cytokine gene is an endogenous or exogenous cytokine gene to the hypoimmunogenic T cells.

In some embodiments, the cytokine gene encodes a pro-inflammatory cytokine.

In some embodiments, the pro-inflammatory cytokine is selected from the group consisting of IL-1, IL-2, IL-9, IL-12, IL-18, TNF, IFN-gamma, and a functional fragment thereof.

In some embodiments, the domain which upon successful signaling of the CAR induces expression of the cytokine gene comprises a transcription factor or functional domain or fragment thereof.

In some embodiments, the CAR comprises a CD3 zeta domain or an immunoreceptor tyrosine-based activation motif (ITAM), or functional variant thereof.

In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAR comprises a (i) an anti-CD19 scFv; (ii) a CD8a hinge and transmembrane domain or functional variant thereof; (iii) a 4-1BB costimulatory domain or functional variant thereof; and (iv) a CD3ζsignaling domain or functional variant thereof. In some embodiments, the CAR comprises a (i) an anti-CD19 scFv, an anti-CD20 scFv, an anti-CD22 scFv, or an anti-BCMA scFv; (ii) a CD8a hinge and transmembrane domain or functional variant thereof; (iii) a 4-1BB costimulatory domain or functional variant thereof; and (iv) a CD3ζsignaling domain or functional variant thereof.

In some embodiments, the hypoimmunogenic T cells comprise reduced expression of an endogenous T cell receptor.

In some embodiments, the hypoimmunogenic T cells comprise reduced expression of cytotoxic T-lymphocyte-associated protein 4 (CTLA4) and/or programmed cell death (PD1).

In some embodiments, the population of the hypoimmunogenic T cells is administered at least 3 days or more after the initial administration, optionally at least 4 days, 5 days, 6 days, 7 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 1 month, 2 months, 3 months, 4 months, 5 months, 6 months, 12 months, 18 months, 24 months, 30 months, 36 months, 48 months, 54 months, or 60 months or more.

In some embodiments, the population of the hypoimmunogenic T cells is administered at least 3 days to at least 7 days or more after the initial administration.

In some embodiments, the population of the hypoimmunogenic T cells is administered least 1 month or more after the initial administration.

In some embodiments, the population of the hypoimmunogenic T cells is administered at least 2 months or more after the initial administration.

In some embodiments, upon the initial and/or subsequent administrations, the population of hypoimmunogenic T cells elicits a reduced level of immune activation or no immune activation in the patient.

In some embodiments, upon the initial and/or subsequent administrations, the population of hypoimmunogenic T cells elicits a reduced level of systemic TH1 activation or no systemic TH1 activation in the patient.

In some embodiments, upon the initial and/or subsequent administrations, the population of hypoimmunogenic T cells elicits a reduced level of immune activation of peripheral blood mononuclear cells (PBMCs) or no immune activation of PBMCs in the patient.

In some embodiments, upon the initial and/or subsequent administrations, the population of hypoimmunogenic T cells elicits a reduced level of donor-specific IgG antibodies or no donor specific IgG antibodies against the hypoimmunogenic cells in the patient.

In some embodiments, upon the initial and/or subsequent administrations, the population of hypoimmunogenic T cells elicits a reduced level of IgM and IgG antibody production or no IgM and IgG antibody production against the hypoimmunogenic cells in the patient.

In some embodiments, upon administration, the population of hypoimmunogenic T cells elicits a reduced level of cytotoxic T cell killing or no cytotoxic T cell killing of the hypoimmunogenic cells of the hypoimmunogenic cells in the patient.

In some embodiments, the population of hypoimmunogenic T cells of the initial administration are no longer present in the patient at the subsequent administration.

In some embodiments, the patient is not administered an immunosuppressive agent at least 3 days or more before or after the initial administration of the population of hypoimmunogenic cells.

In some embodiments, the patient is not administered an immunosuppressive agent at least 3 days or more before or after the initial subsequent administration of the population of hypoimmunogenic cells.

In some embodiments, the hypoimmunogenic T cell is a B2M^{indel/indel}, CIITA^{indel/indel} cell comprising the exogenous CD47 polypeptides and optionally a CAR.

The present disclosure is related to U.S. Provisional Application Ser. No. 63/016,190, filed Apr. 27, 2020, and 63/052,360 filed Jul. 15, 2020, the contents of which are hereby incorporated by references in their entireties. Detailed descriptions of hypoimmunogenic cells, methods of producing thereof, and methods of using thereof are found in WO2016183041 filed May 9, 2015, WO2018132783 filed Jan. 14, 2018, WO2020018615 filed Jul. 17, 2019, and WO2020018620 filed Jul. 17, 2019, the disclosures including the examples, sequence listings and figures are incorporated herein by reference in their entirety.

### BRIEF DESCRIPTION OF THE DRAWINGS

FIG. 1 is a set of ELISPOT images and quantitations from serum of NHPs administered wild-type human iPSCs at days 0, 7, 13, and 7X5 following first injection and days 7 and 13 following re-injection of wild-type human iPSCs. * = p < 0.01 by one-way ANOVA, Bonferroni.

FIG. 2 is a set of ELISPOT images and quantitations from serum of NHPs administered human HIP cells at days 0, 7, 13, and 75 following first injection and days 7 and 13 following re-injection of human HIP cells.

FIG. 3 is a set of cell survival traces of wild-type human iPSCs incubated with PBMC isolated from NHP at days 7 and 13 following re-injection of wild-type human iPSCs.

FIG. 4 is a set of cell survival traces of human HIP cells incubated with PBMC isolated from NHP at days 7 and 13 following re-injection of human HIP cells.

FIG. 5 is a set of cell survival traces of wild-type human iPSCs incubated with cytotoxic T cells (CD3+CD8+) isolated from NHP at days 7 and 13 following re-injection of wild-type human iPSCs.

FIG. 6 is a set of cell survival traces of human HIP cells incubated with cytotoxic T cells (CD3+CD8+) isolated from NHP at days 7 and 13 following re-injection of human HIP cells.

FIG. 7 is a set of graphs showing donor-specific IgM antibody binding in serum of NHPs administered wild-type human iPSCs at days 0, 7, 13, and 75 following first injection and days 7 and 13 following re-injection of wild-type human iPSCs.

FIG. 8 is a set of graphs showing donor-specific IgG antibody binding in serum of NHPs administered wild-type human iPSCs at days 0, 7, 13, and 75 following first injection and days 7 and 13 following re-injection of wild-type human iPSCs.

FIG. 9 is a set of graphs showing donor-specific IgM antibody binding in serum of NHPs administered human HIP cells at days 0, 7, 13, and 75 following first injection and days 7 and 13 following re-injection of human HIP cells.

FIG. 10 is a set of graphs showing donor-specific IgG antibody binding in serum of NHPs administered human HIP cells at days 0, 7, 13, and 75 following first injection and days 7 and 13 following re-injection of human HIP cells.

FIG. 11 is a set of graphs showing total IgM antibodies in serum of NHPs administered wild-type human iPSCs at days 0, 7, 13, and 75 following first injection and days 7 and 13 following re-injection of wild-type human iPSCs.

FIG. 12 is a set of graphs showing total IgG antibodies in serum of NHPs administered wild-type human iPSCs at days 0, 7, 13, and 75 following first injection and days 7 and 13 following re-injection of wild-type human iPSCs.

FIG. 13 is a set of graphs showing total IgM antibodies in serum of NHPs administered human HIP cells at days 0, 7, 13, and 75 following first injection and days 7 and 13 following re-injection of human HIP cells.

FIG. 14 is a set of graphs showing total IgG antibodies in serum of NHPs administered human HIP cells at days 0, 7, 13, and 75 following first injection and days 7 and 13 following re-injection of human HIP cells.

Other objects, advantages and embodiments of the present technology will be apparent from the detailed description following.

### DETAILED DESCRIPTION

### I. Introduction

The present technology is related methods of treat disorders and conditions comprising administering more than one dose of hypoimmunogenic cells. To overcome the problem of a subject’s immune rejection of these stem cell-derived transplants, the inventors have developed and disclose herein a methods for administering hypoimmunogenic cells (e.g., hypoimmunogenic T cells and differentiated cells derived from hypoimmunogenic pluripotent stem cells) that represent a viable source for any transplantable cell type. Such cells are protected from adaptive and innate immune rejection upon administration to a recipient subject. Advantageously, the cells disclosed herein are not rejected by the recipient subject’s immune system, regardless of the subject’s genetic make-up. Such cells are protected from adaptive and innate immune rejection upon administration to a recipient subj ect.

In some embodiments, hypoimmunogenic cells outlined herein are not subject to an innate immune cell rejection. In some instances, hypoimmunogenic ells are not susceptible to NK cell-mediated lysis. In some instances, hypoimmunogenic cells are not susceptible to macrophage engulfment. In some embodiments, hypoimmunogenic cells are useful as a source of universally compatible cells or tissues (e.g., universal donor cells or tissues) that are transplanted into a recipient subject with little to no immunosuppressant agent needed. Such hypoimmunogenic cells retain cell-specific characteristics and features upon transplantation.

In some embodiments, provided herein are methods for treating a disorder comprising administering cells (e.g., primary T cells and differentiated derivatives of stem cells) that evade immune rejection in an MHC-mismatched allogenic recipient. In some instances, differentiated cells produced from the stem cells outlined herein evade immune rejection when repeatedly administered (e.g., transplanted or grafted) to MHC-mismatched allogenic recipient.

In some embodiments, provided herein are cells derived from primary T cells that are hypoimmunogenic and differentiated cells derived from hypoimmunogenic pluripotent stem cells that are also hypoimmunogenic. In some embodiments, such hypoimmunogenic outlined herein have reduced immunogenicity (such as, at least 2.5%-99% less immunogenicity) compared to wild-type immunogeneic cells. In some instances, the hypoimmunogenic T cells lack immunogenicity compared to wild-type T cells. The differentiated derivatives thereof are suitable as universal donor cells for transplantation or engrafting into a recipient patient. In some embodiments, such cells are nonimmunogenic to a subj ect.

In some embodiments, cells disclosed herein fail to elicit a systemic immune response upon administration to a subject. In some cases, the cells do not elicit immune activation of peripheral blood mononuclear cells and serum factors upon administration to a subject. In some instances, the cells do not activate the immune system. In other words, cells described herein exhibit immune evading characteristics and properties. In some embodiments, cells described herein exhibit immunoprivileged characteristics and properties.

### II. Definitions

As used herein to characterize a cell, the term “hypoimmunogenic” generally means that such cell is less prone to immune rejection by a subject into which such cells are transplanted. For example, relative to an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. In some embodiments, genome editing technologies are used to modulate the expression of MHC I and MHC II genes, and thus, generate a hypoimmunogenic cell. In some embodiments, a hypoimmunogenic cell evades immune rejection in an MHC-mismatched allogenic recipient. In some instance, differentiated cells produced from the hypoimmunogenic stem cells outlined herein evade immune rejection when administered (e.g., transplanted or grafted) to an MHC-mismatched allogenic recipient. In some embodiments, a hypoimmunogenic cell is protected from T cell-mediated adaptive immune rejection and/or innate immune cell rejection.

As used herein, the phrases “hypoimmunogenic cells differentiated from hypoimmune induced pluripotent stem cells” and/or “hypoimmunogenic cells differentiated from hypoimmune iPSCs” can be used interchangeably and encompass wherein such differentiated cells including, for example, but are not limited to T cells, neural/neuronal cells, beta cells, hepatocytes, cardiomyocytes, endothelial cells, thyroid cells, pancreatic islet cell types, retinal pigmented epithelium (RPE) cells, and/or any other cells of the subject, e.g., a human subject.

Hypoimmunogencity of a cell can be determined by evaluating the immunogenicity of the cell such as the cell’s ability to elicit adaptive and innate immune responses. Such immune response can be measured using assays recognized by those skilled in the art. In some embodiments, an immune response assay measures the effect of a hypoimmunogenic cell on T cell proliferation, T cell activation, T cell killing, NK cell proliferation, NK cell activation, and macrophage activity. In some cases, hypoimmunogenic cells and derivatives thereof undergo decreased killing by T cells and/or NK cells upon administration to a subject. In some instances, the cells and derivatives thereof show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

“Pluripotent stem cells” as used herein have the potential to differentiate into any of the three germ layers: endoderm (e.g., the stomach linking, gastrointestinal tract, lungs, etc), mesoderm (e.g., muscle, bone, blood, urogenital tissue, etc) or ectoderm (e.g. epidermal tissues and nervous system tissues). The term “pluripotent stem cells,” as used herein, also encompasses “induced pluripotent stem cells”, or “iPSCs”, “embryonic stem cells”, or “ESCs”, a type of pluripotent stem cell derived from a non-pluripotent cell. In some embodiments, a pluripotent stem cell is produced or generated from a cell that is not a pluripotent cell. In other words, pluripotent stem cells can be direct or indirect progeny of a non-pluripotent cell. Examples of parent cell+s include somatic cells that have been reprogrammed to induce a pluripotent, undifferentiated phenotype by various means. Such “ESC”, “ESC”, “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The- generation of induced pluripotent stem cells (iPSCs) is outlined below. As used herein, “hiPSCs” are human induced pluripotent stem cells.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with (β-microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

As used herein, the terms “grafting”, “administering,” “introducing”, “implanting” and “transplanting” as well as grammatical variations thereof are used interchangeably in the context of the placement of cells (e.g. cells described herein) into a subject, by a method or route which results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years. In some embodiments, the cells can also be administered (e.g., injected) a location other than the desired site, such as in the brain or subcutaneously, for example, in a capsule to maintain the implanted cells at the implant location and avoid migration of the implanted cells.

As used herein, the term “treating” and “treatment” includes administering to a subject an effective amount of cells described herein so that the subject has a reduction in at least one symptom of the disease or an improvement in the disease, for example, beneficial or desired clinical results. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a condition, disease or disorder are alleviated by at least 5%, at least 10%, at least 20%, at least 30%, at least 40%, or at least 50% upon treatment of the condition, disease or disorder.

The term “cancer” as used herein is defined as a hyperproliferation of cells whose unique trait (e.g., loss of normal controls) results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and urinary bladder cancer. As used herein, the term “tumor” refers to an abnormal growth of cells or tissues of the malignant type, unless otherwise specifically indicated and does not include a benign type tissue.

The term “chronic infectious disease” refers to a disease caused by an infectious agent wherein the infection has persisted. Such a disease may include hepatitis (A, B, or C), herpes virus (e.g., VZV, HSV-1, HSV-6, HSV-II, CMV, and EBV), and HIV/AIDS. Non-viral examples may include chronic fungal diseases such Aspergillosis, Candidiasis, Coccidioidomycosis, and diseases associated with Cryptococcus and Histoplasmosis. None limiting examples of chronic bacterial infectious agents may be Chlamydia pneumoniae, Listeria monocytogenes, and Mycobacterium tuberculosis. In some embodiments, the disorder is human immunodeficiency virus (HIV) infection. In some embodiments, the disorder is acquired immunodeficiency syndrome (AIDS).

The term “autoimmune disease” refers to any disease or disorder in which the subject mounts a destructive immune response against its own tissues. Autoimmune disorders can affect almost every organ system in the subject (e.g., human), including, but not limited to, diseases of the nervous, gastrointestinal, and endocrine systems, as well as skin and other connective tissues, eyes, blood and blood vessels. Examples of autoimmune diseases include, but are not limited to Hashimoto’s thyroiditis, Systemic lupus erythematosus, Sjogren’s syndrome, Graves’ disease, Scleroderma, Rheumatoid arthritis, Multiple sclerosis, Myasthenia gravis and Diabetes.

In some embodiments, the present technology contemplates treatment of non-sensitized subjects. For example, subjects contemplated for the present treatment methods are not sensitized to or against one or more alloantigens. In some embodiments, the patient is not sensitized from a previous pregnancy or a previous allogeneic transplant (including, for example but not limited to an allogeneic cell transplant, an allogeneic blood transfusion, an allogeneic tissue transplant, and an allogeneic organ transplant). In some embodiments, the one or more alloantigens the patent is not sensitized against comprise human leukocyte antigens. In some embodiments, the patient does not exhibit memory B cells and/or memory T cells reactive against the one or more alloantigens. In some embodiments, sensitization could include sensitization to at least a portion of an autologous CAR T cell, such as the CAR expressed by the autologous T cell, and in the present methods the patient is not sensitized against any portion of such autologous CAR T cells.

In some embodiments, the present technology contemplates altering target polynucleotide sequences in any manner which is available to the skilled artisan, e.g., utilizing a TALEN system or RNA-guided transposases. It should be understood that although examples of methods utilizing CRISPR/Cas (e.g., Cas9 and Cas12A) and TALEN are described in detail herein, the technology is not limited to the use of these methods/systems. Other methods of targeting, e.g., B2M, to reduce or ablate expression in target cells known to the skilled artisan can be utilized herein.

The methods of the present technology can be used to alter a target polynucleotide sequence in a cell. The present technology contemplates altering target polynucleotide sequences in a cell for any purpose. In some embodiments, the target polynucleotide sequence in a cell is altered to produce a mutant cell. As used herein, a “mutant cell” refers to a cell with a resulting genotype that differs from its original genotype. In some instances, a “mutant cell” exhibits a mutant phenotype, for example when a normally functioning gene is altered using the CRISPR/Cas systems. In other instances, a “mutant cell” exhibits a wild-type phenotype, for example when a CRISPR/Cas system is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A CRISPR/Cas system can be used to induce an indel of any length or a point mutation in a target polynucleotide sequence.

As used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use the CRISPR/Cas systems to knock out a target polynucleotide sequence or a portion thereof based upon the details described herein.

In some embodiments, the alteration results in a knock out of the target polynucleotide sequence or a portion thereof. Knocking out a target polynucleotide sequence or a portion thereof using a CRISPR/Cas system can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

By “knock in” herein is meant a process that adds a genetic function to a host cell. This causes increased levels of the knocked in gene product, e.g., an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

In some embodiments, the alteration results in reduced expression of the target polynucleotide sequence. The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “exogenous” in intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell.

The term “endogenous” refers to a referenced molecule or polypeptide that is present in the cell. Similarly, the term when used in reference to expression of an encoding nucleic acid refers to expression of an encoding nucleic acid contained within the cell and not exogenously introduced.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat′1. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states, which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the present technology. Any recited method may be carried out in the order of events recited or in any other order that is logically possible. Although any methods and materials similar or equivalent to those described herein may also be used in the practice or testing of the present technology, representative illustrative methods and materials are now described.

As described in the present technology, the following terms will be employed, and are defined as indicated below.

Before the present technology is further described, it is to be understood that this technology is not limited to some embodiments described, as such may, of course, vary. It is also to be understood that the terminology used herein is for the purpose of describing some embodiments only, and is not intended to be limiting, since the scope of the present technology will be limited only by the appended claims.

Unless defined otherwise, all technical and scientific terms used herein have the same meaning as commonly understood by one of ordinary skill in the art to which this technology belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the present technology. The upper and lower limits of these smaller ranges may independently be included in the smaller ranges and are also encompassed within the present technology, subject to any specifically excluded limit in the stated range. Where the stated range includes one or both of the limits, ranges excluding either or both of those included limits are also included in the present technology. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the present technology described herein is not entitled to antedate such publication by virtue of prior technology. Further, the dates of publication provided might be different from the actual publication dates, which may need to be independently confirmed.

### III. Detailed Description of the Embodiments

### A. Methods for Administering Hypoimmunogenic Cells

As is described in further detail herein, provided herein are methods for treating a patient with a disorder through multiple administrations of cells, particularly hypoimmunogenic cells derived from primary T cells and hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells). As will be appreciated, for all the multiple embodiments described herein related to the timing and/or combinations of therapies, the administering of the cells are accomplished by a method or route which results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable.

Provided herein are methods for treating a patient with a disorder comprising providing at least two administrations of a population(s) of hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) to the patient. In some embodiments, the method comprises providing at least two administrations of a population(s) of hypoimmunogenic cells derived from primary T cells. In some embodiments, the first administration of hypoimmunogenic cells is provided to the patient at least 1 month (e.g., 1 month, 2 months, 3 months, 4 months, 5 months, 6 months, or more) or more before the second administration of hypoimmunogenic cells. In other embodiments, the first administration of hypoimmunogenic cells is provided to the patient at least 2 months (e.g., 2 months, 3 months, 4 months, 5 months, 6 months, or more) or more before the second administration of hypoimmunogenic cells. In certain embodiments, the first administration of hypoimmunogenic cells is provided to the patient at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more before the second administration of hypoimmunogenic cells. In certain embodiments, the first administration of hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) is provided to the patient at least 3 days, 4 days, 5 days, 6 days, or 7 days or more before the second administration of hypoimmunogenic cells. In many embodiments, the first population of hypoimmunogenic cells differentiated from hypoimmune iPSCs (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) and the second population of hypoimmunogenic cells differentiated from hypoimmune iiPSCs (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) comprises the same cell types. In some embodiments, the first population of cells and the second population of hypoimmunogenic cells differentiated from hypoimmune iPSCs comprises the same cell types. In some embodiments, the first population of hypoimmunogenic cells differentiated from hypoimmune iPSCs and the second population of hypoimmunogenic cells differentiated from hypoimmune iPSCs comprises the same percentages of cell types. In other embodiments, the first population of hypoimmunogenic cells differentiated from hypoimmune iPSCs and the second population of hypoimmunogenic cells differentiated from hypoimmune iPSCs comprises different percentages of cell types. In certain embodiments, the first administration of hypoimmunogenic cells derived from primary T cells is provided to the patient at least 3 days, 4 days, 5 days, 6 days, or 7 days or more before the second administration of hypoimmunogenic cells. In many embodiments, the first population of hypoimmunogenic cells derived from primary T cells and the second population of hypoimmunogenic derived from primary T cells comprises the same cell types. In some embodiments, the first population of cells and the second population of hypoimmunogenic cells derived from primary T cells comprises the same cell types. In some embodiments, the first population of hypoimmunogenic cells derived from primary T cells and the second population of hypoimmunogenic cells derived from primary T cells comprises the same percentages of cell types. In other embodiments, the first population of hypoimmunogenic cells derived from primary T cells and the second population of hypoimmunogenic cells derived from primary T cells comprises different percentages of cell types.

In some embodiments, an immunosuppressive and/or immunomodulatory agent is not administered to the patient before the first administration of the population of hypoimmunogenic cells derived from primary T cells or the population of hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells). In many embodiments, an immunosuppressive and/or immunomodulatory agent is administered to the patient before the first administration of the population of hypoimmunogenic cells derived from primary T cells or the population of hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells). In some embodiments, an immunosuppressive and/or immunomodulatory agent is administered at least 1, 2, 3, 4, 5, 6, 7, 8, 9, 10, 11, 12, 13, 14 days or more before the first administration of the hypoimmunogenic cells, such as but not limited to, the hypoimmunogenic cells derived from primary T cells or the hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells). In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first administration of the hypoimmunogenic cells, such as but not limited to, the hypoimmunogenic cells derived from primary T cells or the hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells). In some embodiments, an immunosuppressive and/or immunomodulatory agent is not administered to the patient after the first administration of the hypoimmunogenic cells, such as but not limited to, the hypoimmunogenic cells derived from primary T cells or the hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells), or is administered at least 1, 2, 3, 4, 5, 6, 7, 8, 9, 10, 11, 12, 13, 14 days or more after the first administration of the hypoimmunogenic cells, such as but not limited to, the hypoimmunogenic cells derived from primary T cells or the hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells). In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first administration of the hypoimmunogenic cells, such as but not limited to, the hypoimmunogenic cells derived from primary T cells or the hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells). Non-limiting examples of an immunosuppressive and/or immunomodulatory agent include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-a and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40, CD45, IFN-gamma, TNF-.alpha., IL-4, IL-5, IL-6R, IL-6, IGF, IGFR1, IL-7, IL-8, IL-10, CD1 1a,or CD58, and antibodies binding to any of their ligands. In some embodiments where an immunosuppressive and/or immunomodulatory agent is administered to the patient before or after the first administration of the hypoimmunogenic cells, such as but not limited to, the hypoimmunogenic cells derived from primary T cells or the hypoimmunogenic cells differentiated from hypoimmune iPSCs, the administration is at a lower dosage than would be required for hypoimmunogenic cells, such as but not limited to, hypoimmunogenic cells derived from primary T cells or hypoimmunogenic cells differentiated from differentiated from hypoimmune iPSCs with MHC I and/or MHC II expression and without exogenous expression of CD47. In some embodiments where an immunosuppressive and/or immunomodulatory agent is administered to the patient before or after the first administration of the cells, the administration is at a lower dosage than would be required for hypoimmunogenic cells, such as but not limited to, the hypoimmunogenic cells derived from primary T cells or hypoimmunogenic cells differentiated from hypoimmune iPSCs with MHC I and/or MHC II expression and without exogenous expression of CD24.

In one embodiment, such an immunosuppressive and/or immunomodulatory agent may be selected from soluble IL-15R, IL-10, B7 molecules (e.g., B7-1, B7-2, variants thereof, and fragments thereof), ICOS, and OX40, an inhibitor of a negative T cell regulator (such as an antibody against CTLA4) and similar agents.

In some embodiments, an immunosuppressive and/or immunomodulatory agent is not administered to the patient before the second administration of the population of hypoimmunogenic cells, such as but not limited to, hypoimmunogenic cells derived from primary T cells or hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells). In many embodiments, an immunosuppressive and/or immunomodulatory agent is administered to the patient before the second administration of the population of hypoimmunogenic cells, such as but not limited to, hypoimmunogenic cells derived from primary T cells or hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells). In some embodiments, an immunosuppressive and/or immunomodulatory agent is administered at least 1, 2, 3, 4, 5, 6, 7, 8, 9, 10, 11, 12, 13, 14 days or more before the second administration of the hypoimmunogenic cells, such as but not limited to, hypoimmunogenic cells derived from primary T cells or hypoimmunogenic cells differentiated from hypoimmune iPSCs. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the second administration of the hypoimmunogenic cells, such as but not limited to, hypoimmunogenic cells derived from primary T cells or hypoimmunogenic cells differentiated from hypoimmune iPSCs. In some embodiments, an immunosuppressive and/or immunomodulatory agent is not administered to the patient after the second administration of the cells or is administered at least 1, 2, 3, 4, 5, 6, 7, 8, 9, 10, 11, 12, 13, 14 days or more after the second administration of the hypoimmunogenic cells, such as but not limited to, hypoimmunogenic cells derived from primary T cells or hypoimmunogenic cells differentiated from hypoimmune iPSCs. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the second administration of the hypoimmunogenic cells, such as but not limited to, the hypoimmunogenic cells derived from primary T cells or the hypoimmunogenic cells differentiated from hypoimmune iPSCs. In some embodiments where an immunosuppressive and/or immunomodulatory agent is administered to the patient before or after the second administration of the hypoimmunogenic cells, such as but not limited to, the hypoimmunogenic cells derived from primary T cells or the hypoimmunogenic cells differentiated from hypoimmune iPSCs, the administration is at a lower dosage than would be required for hypoimmunogenic cells, such as but not limited to, the hypoimmunogenic cells derived from primary T cells or the hypoimmunogenic cells differentiated from hypoimmune induced pluripotent stem cells with MHC I and/or MHC II expression and without exogenous expression of CD47. In some embodiments where an immunosuppressive and/or immunomodulatory agent is administered to the patient before or after the second administration of the hypoimmunogenic cells, such as but not limited to, the hypoimmunogenic cells derived from primary T cells or the hypoimmunogenic cells differentiated from hypoimmune iPSCs, the administration is at a lower dosage than would be required for cells with MHC I and/or MHC II expression and without exogenous expression of CD24.

In some embodiments, the treatment method comprises administering a first population of hypoimmunogenic cells, such as but not limited to, the hypoimmunogenic cells derived from primary T cells or the hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) to a patient, followed by a recovery period wherein the patient is not administered hypoimmunogenic cells, such as but not limited to, the hypoimmunogenic cells derived from primary T cells or the hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells), and then administering a second population of hypoimmunogenic cells, such as but not limited to, the hypoimmunogenic cells derived from primary T cells or the hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) to the patient. In some embodiments, the recovery period comprises at least 1 month (e.g., 1 month, 2 months, 3 months, 4 months, 5 months, 6 months, 12 months, 18 months, 24 months, 30 months, 36 months, 48 months, 54 months, or 60 months or more) or more. In certain embodiments, the recovery period comprises at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more. In certain embodiments, the recovery period comprises at least 3 days, 4 days, 5 days, 6 days, 7 days, or 1 week. In certain embodiments, the recovery period comprises at least 3 days. In some embodiments, the recovery period begins following the first administration of the population of hypoimmunogenic cells derived from primary T cells or the population of hypoimmunogenic cells differentiated from hypoimmune iPSCs and ends when such cells are no longer present or detectable in the patient. In some embodiments, the recovery period is based on the administration route. In some embodiments, the recovery period comprises about 3 days when the administration route is intramuscular. In some embodiments, the recovery period is based on the timing of administration of a further therapy. In some embodiments, the recovery period is based on the timing of administration of a further therapy wherein in the further therapy does not comprise hypoimmunogenic cells, such as but not limited to, hypoimmunogenic cells derived from primary T cells or hypoimmunogenic cells differentiated from hypoimmune iPSCs.

In some embodiments, the first population of hypoimmunogenic cells differentiated from hypoimmune iPSCs administered to a patient is the same as the second population of hypoimmunogenic cells differentiated from hypoimmune iPSCs administered to the patient. In some embodiments, the first population of hypoimmunogenic cells differentiated from hypoimmune iPSCs administered to a patient is different from the second population of hypoimmunogenic cells differentiated from hypoimmune iPSCs administered to the patient. In some embodiments, the second population of hypoimmunogenic cells differentiated from hypoimmune iPSCs administered to the patient is a mixture of the first population of hypoimmunogenic cells administered to the patient and a different population of hypoimmunogenic cells differentiated from hypoimmune iPSCs. In some embodiments, the first population of hypoimmunogenic cells differentiated from hypoimmune iPSCs comprises T lymphocytes (T cells). In some embodiments, the second population of hypoimmunogenic cells differentiated from hypoimmune iPSCs comprises T lymphocytes (T cells). In some embodiments, the first population of hypoimmunogenic cells derived from primary T cells administered to a patient is the same as the second population of hypoimmunogenic cells derived from primary T cells administered to the patient. In some embodiments, the first population of hypoimmunogenic cells derived from primary T cells administered to a patient is different from the second population of hypoimmunogenic cells derived from primary T cells administered to the patient. In some embodiments, the second population of hypoimmunogenic cells derived from primary T cells administered to the patient is a mixture of the first population of hypoimmunogenic cells administered to the patient and a different population of hypoimmunogenic cells derived from primary T cells. In some embodiments, the number of hypoimmunogenic cells such as but not limited to, hypoimmunogenic cells derived from primary T cells or hypoimmunogenic cells differentiated from hypoimmune iPSCs administered in the first population dose is the same as the number of hypoimmunogenic cells (such as but not limited to, hypoimmunogenic cells derived from primary T cells or hypoimmunogenic cells differentiated from hypoimmune iPSCs) administered in the second population dose. In some embodiments, the number of hypoimmunogenic cells (such as but not limited to, hypoimmunogenic cells derived from primary T cells or hypoimmunogenic cells differentiated from hypoimmune iPSCs) administered in the first population dose is less than the number of hypoimmunogenic cells (such as but not limited to, hypoimmunogenic cells derived from primary T cells or hypoimmunogenic cells differentiated from hypoimmune iPSCs) administered in the second population dose. In some embodiments, the number of hypoimmunogenic cells (such as but not limited to, hypoimmunogenic cells derived from primary T cells or hypoimmunogenic cells differentiated from hypoimmune iPSCs) administered in the first population dose is more than the number of hypoimmunogenic cells (such as but not limited to, hypoimmunogenic cells derived from primary T cells or hypoimmunogenic cells differentiated from hypoimmune iPSCs) administered in the second population dose.

In some embodiments, the first population of hypoimmunogenic cells differentiated from hypoimmune induced pluripotent stem cells are selected from the group consisting of T cells, neural/neuronal cells, beta cells, hepatocytes, cardiomyocytes, endothelial cells, thyroid cells, pancreatic islet cell types, and/or retinal pigmented epithelium (RPE) cells. In some embodiments, the second population of hypoimmunogenic cells differentiated from hypoimmune induced pluripotent stem cells are selected from the group consisting of T cells, neural/neuronal cells, beta cells, hepatocytes, cardiomyocytes, endothelial cells, thyroid cells, pancreatic islet cell types, and/or retinal pigmented epithelium (RPE) cells. In some embodiments, the number of hypoimmunogenic cells differentiated from hypoimmune iPSCs administered in the first population dose is the same as the number of hypoimmunogenic cells differentiated from hypoimmune iPSCs administered in the second population dose. In some embodiments, the number of hypoimmunogenic cells differentiated from hypoimmune iPSCs administered in the first population dose is less than the number of hypoimmunogenic cells differentiated from hypoimmune iPSCs administered in the second population dose. In some embodiments, the number of hypoimmunogenic cells differentiated from hypoimmune iPSCs administered in the first population dose is more than the number of hypoimmunogenic cells differentiated from hypoimmune iPSCs administered in the second population dose.

In some embodiments, the first population of hypoimmunogenic cells differentiated from hypoimmune iPSCs are T cells and the second population of hypoimmunogenic cells differentiated from hypoimmune iPSCs are selected from the group consisting of neural/neuronal cells, beta cells, hepatocytes, cardiomyocytes, endothelial cells, thyroid cells, pancreatic islet cell types, and/or retinal pigmented epithelium (RPE) cells. In some embodiments, the first population of hypoimmunogenic cells differentiated from hypoimmune iPSCs are selected from the group consisting of neural/neuronal cells, beta cells, hepatocytes, cardiomyocytes, endothelial cells, thyroid cells, pancreatic islet cell types, and/or retinal pigmented epithelium (RPE) cells and the second population of hypoimmunogenic cells differentiated from hypoimmune iPSCs are T cells. In some embodiments, the first population of hypoimmunogenic cells differentiated from hypoimmune iPSCs are T cells and the second population of hypoimmunogenic cells differentiated from hypoimmune iPSCs are beta cells. In some embodiments, the first population of hypoimmunogenic cells differentiated from hypoimmune iPSCs are T cells and the second population of hypoimmunogenic cells differentiated from hypoimmune iPSCs are hepatocytes. In some embodiments, the first population of hypoimmunogenic cells differentiated from hypoimmune iPSCs are T cells and the second population of hypoimmunogenic cells differentiated from hypoimmune iPSCs are cardiomyocytes. In some embodiments, the first population of hypoimmunogenic cells differentiated from hypoimmune iPSCs are T cells and the second population of hypoimmunogenic cells differentiated from hypoimmune iPSCs are endothelial cells. In some embodiments, the first population of hypoimmunogenic cells differentiated from hypoimmune iPSCs are T cells and the second population of hypoimmunogenic cells differentiated from hypoimmune iPSCs are thyroid cells. In some embodiments, the first population of hypoimmunogenic cells differentiated from hypoimmune iPSCs are T cells and the second population of hypoimmunogenic cells differentiated from hypoimmune iPSCs are pancreatic islet cell types. In some embodiments, the first population of hypoimmunogenic cells differentiated from hypoimmune iPSCs are T cells and the second population of hypoimmunogenic cells differentiated from hypoimmune iPSCs are retinal pigmented epithelium (RPE) cells. In some embodiments, the first population of hypoimmunogenic cells differentiated from hypoimmune iPSCs comprises T cells and the second population of hypoimmunogenic cells differentiated from hypoimmune iPSCs comprises T cells. In some embodiments, the number of hypoimmunogenic cells differentiated from hypoimmune iPSCs administered in the first population dose is the same as the number of hypoimmunogenic cells differentiated from hypoimmune iPSCs administered in the second population dose. In some embodiments, the number of hypoimmunogenic cells differentiated from hypoimmune iPSCs administered in the first population dose is less than the number of hypoimmunogenic cells differentiated from hypoimmune iPSCs administered in the second population dose. In some embodiments, the number of hypoimmunogenic cells differentiated from hypoimmune iPSCs administered in the first population dose is more than the number of hypoimmunogenic cells differentiated from hypoimmune iPSCs administered in the second population dose.

In some embodiments, the first population of hypoimmunogenic cells differentiated from hypoimmune iPSCs comprises T cells and optionally one or more other hypoimmunogenic cells differentiated from hypoimmune iPSCs selected from the group consisting of neural/neuronal cells, beta cells, hepatocytes, cardiomyocytes, endothelial cells, thyroid cells, pancreatic islet cell types, and/or retinal pigmented epithelium (RPE) cells. In some embodiments, the second population of hypoimmunogenic cells differentiated from hypoimmune iPSCs comprises T cells and optionally one or more other hypoimmunogenic cells differentiated from hypoimmune iPSCs selected from the group consisting of neural/neuronal cells, beta cells, hepatocytes, cardiomyocytes, endothelial cells, thyroid cells, pancreatic islet cell types, and/or retinal pigmented epithelium (RPE) cells. In some embodiments, the first population of hypoimmunogenic cells differentiated from hypoimmune iPSCs are T cells and the second population of hypoimmunogenic cells differentiated from hypoimmune iPSCs are neural/neuronal cells. In some embodiments, the first population of hypoimmunogenic cells differentiated from hypoimmune iPSCs are T cells and the second population of hypoimmunogenic cells differentiated from hypoimmune iPSCs are beta cells. In some embodiments, the first population of hypoimmunogenic cells differentiated from hypoimmune iPSCs are T cells and the second population of hypoimmunogenic cells differentiated from hypoimmune iPSCs are hepatocytes. In some embodiments, the first population of hypoimmunogenic cells differentiated from hypoimmune iPSCs are T cells and the second population of hypoimmunogenic cells differentiated from hypoimmune iPSCs are cardiomyocytes. In some embodiments, the first population of hypoimmunogenic cells differentiated from hypoimmune iPSCs are T cells and the second population of hypoimmunogenic cells differentiated from hypoimmune iPSCs are endothelial cells. In some embodiments, the first population of hypoimmunogenic cells differentiated from hypoimmune iPSCs are T cells and the second population of hypoimmunogenic cells differentiated from hypoimmune iPSCs are thyroid cells. In some embodiments, the first population of hypoimmunogenic cells differentiated from hypoimmune iPSCs are T cells and the second population of hypoimmunogenic cells differentiated from hypoimmune iPSCs are pancreatic islet cell types. In some embodiments, the first population of hypoimmunogenic cells differentiated from hypoimmune iPSCs are T cells and the second population of hypoimmunogenic cells differentiated from hypoimmune iPSCs are RPE cells. In some embodiments, the number of hypoimmunogenic cells differentiated from hypoimmune iPSCs administered in the first population dose is the same as the number of hypoimmunogenic cells differentiated from hypoimmune iPSCs administered in the second population dose. In some embodiments, the number of hypoimmunogenic cells differentiated from hypoimmune iPSCs administered in the first population dose is less than the number of hypoimmunogenic cells differentiated from hypoimmune iPSCs administered in the second population dose. In some embodiments, the number of hypoimmunogenic cells differentiated from hypoimmune iPSCs administered in the first population dose is more than the number of hypoimmunogenic cells differentiated from hypoimmune iPSCs administered in the second population dose.

In some embodiments, the first population of hypoimmunogenic cells differentiated from hypoimmune iPSCs comprises T cells and optionally one or more other hypoimmunogenic cells differentiated from hypoimmune iPSCs selected from the group consisting of neural/neuronal cells, beta cells, hepatocytes, cardiomyocytes, endothelial cells, thyroid cells, pancreatic islet cell types, and/or retinal pigmented epithelium (RPE) cells and the second population of hypoimmunogenic cells differentiated from hypoimmune iPSCs are neural/neuronal cells. In some embodiments, the first population of hypoimmunogenic cells differentiated from hypoimmune iPSCs comprises T cells and optionally one or more other hypoimmunogenic cells differentiated from hypoimmune iPSCs selected from the group consisting of neural/neuronal cells, beta cells, hepatocytes, cardiomyocytes, endothelial cells, thyroid cells, pancreatic islet cell types, and/or retinal pigmented epithelium (RPE) cells and the second population of hypoimmunogenic cells differentiated from hypoimmune iPSCs are beta cells. In some embodiments, the first population of hypoimmunogenic cells differentiated from hypoimmune iPSCs comprises T cells and optionally one or more other hypoimmunogenic cells differentiated from hypoimmune iPSCs selected from the group consisting of neural/neuronal cells, beta cells, hepatocytes, cardiomyocytes, endothelial cells, pancreatic islet cell types, and/or retinal pigmented epithelium (RPE) cells and the second population of hypoimmunogenic cells differentiated from hypoimmune iPSCs are hepatocytes. In some embodiments, the first population of hypoimmunogenic cells differentiated from hypoimmune iPSCs comprises T cells and optionally one or more other hypoimmunogenic cells differentiated from hypoimmune iPSCs selected from the group consisting of neural/neuronal cells, beta cells, hepatocytes, cardiomyocytes, endothelial cells, thyroid cells, pancreatic islet cell types, and/or retinal pigmented epithelium (RPE) cells and the second population of hypoimmunogenic cells differentiated from hypoimmune iPSCs are cardiomyocytes. In some embodiments, the first population of hypoimmunogenic cells differentiated from hypoimmune iPSCs comprises T cells and optionally one or more other hypoimmunogenic cells differentiated from hypoimmune iPSCs selected from the group consisting of neural/neuronal cells, beta cells, hepatocytes, cardiomyocytes, endothelial cells, thyroid cells, pancreatic islet cell types, and/or retinal pigmented epithelium (RPE) cells and the second population of hypoimmunogenic cells differentiated from hypoimmune iPSCs are endothelial cells. In some embodiments, the first population of hypoimmunogenic cells differentiated from hypoimmune iPSCs comprises T cells and optionally one or more other hypoimmunogenic cells differentiated from hypoimmune iPSCs selected from the group consisting of neural/neuronal cells, beta cells, hepatocytes, cardiomyocytes, endothelial cells, thyroid cells, pancreatic islet cell types, and/or retinal pigmented epithelium (RPE) cells and the second population of hypoimmunogenic cells differentiated from hypoimmune iPSCs are thyroid cells. In some embodiments, the first population of hypoimmunogenic cells differentiated from hypoimmune iPSCs comprises T cells and optionally one or more other hypoimmunogenic cells differentiated from hypoimmune iPSCs selected from the group consisting of neural/neuronal cells, beta cells, hepatocytes, cardiomyocytes, endothelial cells, thyroid cells, pancreatic islet cell types, and/or retinal pigmented epithelium (RPE) cells and the second population of hypoimmunogenic cells differentiated from hypoimmune iPSCs are pancreatic islet cell types. In some embodiments, the first population of hypoimmunogenic cells differentiated from hypoimmune iPSCs comprises T cells and optionally one or more other hypoimmunogenic cells differentiated from hypoimmune iPSCs selected from the group consisting of neural/neuronal cells, beta cells, hepatocytes, cardiomyocytes, endothelial cells, thyroid cells, pancreatic islet cell types, and/or retinal pigmented epithelium (RPE) cells and the second population of hypoimmunogenic cells differentiated from hypoimmune iPSCs are retinal pigmented epithelium (RPE) cells. In some embodiments, the first population of hypoimmunogenic cells differentiated from hypoimmune iPSCs comprises T cells and optionally one or more other hypoimmunogenic cells differentiated from hypoimmune iPSCs selected from the group consisting of neural/neuronal cells, beta cells, hepatocytes, cardiomyocytes, endothelial cells, thyroid cells, pancreatic islet cell types, and/or retinal pigmented epithelium (RPE) cells and the second population of hypoimmunogenic cells differentiated from hypoimmune iPSCs comprises T cells. In some embodiments, the number of hypoimmunogenic cells differentiated from hypoimmune iPSCs administered in the first population dose is the same as the number of hypoimmunogenic cells differentiated from hypoimmune iPSCs administered in the second population dose. In some embodiments, the number of hypoimmunogenic cells differentiated from hypoimmune iPSCs administered in the first population dose is less than the number of hypoimmunogenic cells differentiated from hypoimmune iPSCs administered in the second population dose. In some embodiments, the number of hypoimmunogenic cells differentiated from hypoimmune iPSCs administered in the first population dose is more than the number of hypoimmunogenic cells differentiated from hypoimmune iPSCs administered in the second population dose.

In some embodiments, the first population comprises T cells derived from hypoimmunogenic cells differentiated from hypoimmune iPSCs and are a mixture of CD4+ and CD8+ cells. In some embodiments, the first population comprises T cells derived from hypoimmunogenic cells differentiated from hypoimmune iPSCs and are CD4+ cells. In some embodiments the first population comprises T cells derived from hypoimmunogenic cells differentiated from hypoimmune iPSCs and are CD8+ cells. In some embodiments, the first population comprises T cells derived from hypoimmunogenic cells differentiated from hypoimmune iPSCs and are non-activated T cells. In some embodiments, the second population comprises T cells derived from hypoimmunogenic cells differentiated from hypoimmune iPSCs and are a mixture of CD4+ and CD8+ cells. In some embodiments, the second population comprises T cells derived from hypoimmunogenic cells differentiated from hypoimmune iPSCs and are CD4+ cells. In some embodiments, the second population comprises T cells derived from hypoimmunogenic cells differentiated from hypoimmune iPSCs and are CD8+ cells. In some embodiments, the second population comprises T cells derived from hypoimmunogenic cells differentiated from hypoimmune iPSCs and are non-activated T cells. In some embodiments, the first and second populations comprise T cells derived from hypoimmunogenic cells differentiated from hypoimmune iPSCs and are a mixture of CD4+ and/or CD8+ cells. In some embodiments, the first and second populations comprise T cells derived from hypoimmunogenic cells differentiated from hypoimmune iPSCs and are CD4+ cells. In some embodiments, the first and second populations comprise T cells derived from hypoimmunogenic cells differentiated from hypoimmune iPSCs and are CD8+ cells. In some embodiments, the first population comprises T cells derived from hypoimmunogenic cells differentiated from hypoimmune iPSCs and are CD4+ and the second population comprises T cells derived from hypoimmunogenic cells differentiated from hypoimmune iPSCs and are CD8+ cells. In some embodiments, the first population comprises T cells derived from hypoimmunogenic cells differentiated from hypoimmune iPSCs and are CD8+ and the second population comprises T cells derived from hypoimmunogenic cells differentiated from hypoimmune iPSCs and are CD4+ cells. In some embodiments, the number of hypoimmunogenic cells differentiated from hypoimmune iPSCs administered in the first population dose is the same as the number of hypoimmunogenic cells differentiated from hypoimmune iPSCs administered in the second population dose. In some embodiments, the number of hypoimmunogenic cells differentiated from hypoimmune iPSCs administered in the first population dose is less than the number of hypoimmunogenic cells differentiated from hypoimmune iPSCs administered in the second population dose. In some embodiments, the number of hypoimmunogenic cells differentiated from hypoimmune iPSCs administered in the first population dose is more than the number of hypoimmunogenic cells differentiated from hypoimmune iPSCs administered in the second population dose.

In some embodiments, the first population comprises hypoimmunogenic T cells derived from primary T cells and are a mixture of CD4+ and CD8+ cells. In some embodiments, the first population comprises hypoimmunogenic T cells derived from primary T cells and are CD4+ cells. In some embodiments the first population comprises hypoimmunogenic T cells derived from primary T cells and are CD8+ cells. In some embodiments, the first population comprises hypoimmunogenic T cells derived from primary T cells and are non-activated T cells. In some embodiments, the second population comprises hypoimmunogenic T cells derived from primary T cells and are a mixture of CD4+ and CD8+ cells. In some embodiments, the second population comprises hypoimmunogenic T cells derived from primary T cells and are CD4+ cells. In some embodiments, the second population comprises hypoimmunogenic T cells derived from primary T cells and are CD8+ cells. In some embodiments, the second population comprises hypoimmunogenic T cells derived from primary T cells and are non-activated T cells. In some embodiments, the first and second populations comprise hypoimmunogenic T cells derived from primary T cells and are a mixture of CD4+ and/or CD8+ cells. In some embodiments, the first and second populations comprise hypoimmunogenic T cells derived from primary T cells and are CD4+ cells. In some embodiments, the first and second populations comprise hypoimmunogenic T cells derived from primary T cells and are CD8+ cells. In some embodiments, the first population comprises hypoimmunogenic T cells derived from primary T cells and are CD4+ and the second population comprises hypoimmunogenic T cells derived from primary T cells and are CD8+ cells. In some embodiments, the first population comprises hypoimmunogenic T cells derived from primary T cells and are CD8+ and the second population comprises hypoimmunogenic T cells derived from primary T cells and are CD4+ cells. In some embodiments, the number of hypoimmunogenic T cells derived from primary T cells administered in the first population dose is the same as the number of hypoimmunogenic T cells derived from primary T cells administered in the second population dose. In some embodiments, the hypoimmunogenic T cells derived from primary T cells administered in the first population dose is less than the number of hypoimmunogenic T cells derived from primary T cells administered in the second population dose. In some embodiments, the number of hypoimmunogenic T cells derived from primary T cells administered in the first population dose is more than the number of hypoimmunogenic T cells derived from primary T cells administered in the second population dose.

In some embodiments, the administered population of hypoimmunogenic cells such as but not limited to, hypoimmunogenic cells derived from primary T cells or hypoimmunogenic cells differentiated from hypoimmune induced pluripotent stem cells elicits a decreased or lower level of immune activation in the patient. In some instances, the level of immune activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation produced by the administration of immunogenic cells. In some embodiments, the administered population of hypoimmunogenic cells such as but not limited to, hypoimmunogenic cells derived from primary T cells or hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) fails to elicit immune activation in the patient.

In some embodiments, the administered population of hypoimmunogenic cells such as but not limited to, hypoimmunogenic cells derived from primary T cells or hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) elicits a decreased or lower level of systemic TH1 activation in the patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells. In some embodiments, the administered population of hypoimmunogenic cells such as but not limited to, hypoimmunogenic cells derived from primary T cells or hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) fails to elicit systemic TH1 activation in the patient.

In some embodiments, the administered population of hypoimmunogenic cells such as but not limited to, hypoimmunogenic cells derived from primary T cells or hypoimmunogenic cells differentiated from hypoimmune induced pluripotent stem cells elicits a decreased or lower level of immune activation of peripheral blood mononuclear cells (PBMCs) in the patient. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells. In some embodiments, the administered population of hypoimmunogenic cells such as but not limited to, hypoimmunogenic cells derived from primary T cells or hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) fails to elicit immune activation of PBMCs in the patient.

In some embodiments, the administered population of hypoimmunogenic cells such as but not limited to, hypoimmunogenic cells derived from primary T cells or hypoimmunogenic cells differentiated from hypoimmune induced pluripotent stem cells elicits a decreased or lower level of donor-specific IgG antibodies in the patient. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells. In some embodiments, the administered population of hypoimmunogenic cells such as but not limited to, hypoimmunogenic cells derived from primary T cells or hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) fails to elicit donor-specific IgG antibodies in the patient.

In some embodiments, the administered population of hypoimmunogenic cells such as but not limited to, hypoimmunogenic cells derived from primary T cells or hypoimmunogenic cells differentiated from hypoimmune induced pluripotent stem cells elicits a decreased or lower level of IgM and IgG antibody production in the patient. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells. In some embodiments, the administered population of hypoimmunogenic cells such as but not limited to, hypoimmunogenic cells derived from primary T cells or hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) fails to elicit IgM and IgG antibody production in the patient.

In some embodiments, the administered population of hypoimmunogenic cells such as but not limited to, hypoimmunogenic cells derived from primary T cells or hypoimmunogenic cells differentiated from hypoimmune induced pluripotent stem cells elicits a decreased or lower level of cytotoxic T cell killing in the patient. In some instances, the level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells. In some embodiments, the administered population of hypoimmunogenic cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) fails to elicit cytotoxic T cell killing in the patient.

### B. Chimeric Antigen Receptors

Provided herein are hypoimmunogenic cells, including hypoimmunogenic cells differentiated from hypoimmune induced pluripotent stem cells and hypoimmunogenic cells derived from primary T cells, comprising a chimeric antigen receptor (CAR). In some embodiments, the CAR is selected from the group consisting of a first generation CAR, a second generation CAR, a third generation CAR, and a fourth generation CAR.

In some embodiments, a hypoimmunogenic cell described herein comprises a polynucleotide encoding a chimeric antigen receptor (CAR) comprising an antigen binding domain. In some embodiments, a hypoimmunogenic cell described herein comprises a chimeric antigen receptor (CAR) comprising an antigen binding domain. In some embodiments, the polynucleotide is or comprises a chimeric antigen receptor (CAR) comprising an antigen binding domain. In some embodiments, the CAR is or comprises a first generation CAR comprising an antigen binding domain, a transmembrane domain, and at least one signaling domain (e.g., one, two or three signaling domains). In some embodiments, the CAR comprises a second generation CAR comprising an antigen binding domain, a transmembrane domain, and at least two signaling domains. In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

### 1. Antigen Binding Domain (ABD) Targets an Antigen Characteristic of a Neoplastic Or Cancer Cell

In some embodiments, the antigen binding domain (ABD) targets an antigen characteristic of a neoplastic cell. In other words, the antigen binding domain targets an antigen expressed by a neoplastic or cancer cell. In some embodiments, the ABD binds a tumor associated antigen. In some embodiments, the antigen characteristic of a neoplastic cell (e.g., antigen associated with a neoplastic or cancer cell) or a tumor associated antigen is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/ threonine kinase, receptor guanylyl cyclase, histidine kinase associated receptor, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (S1P1R), NMDA channel, transmembrane protein, multispan transmembrane protein, T-cell receptor motifs; T-cell alpha chains; T-cell β chains; T-cell γ chains; T-cell δ chains; CCR7; CD3; CD4; CD5; CD7; CD8; CD11b; CD11c; CD16; CD19; CD20; CD21 ; CD22; CD25; CD28; CD34; CD35; CD40; CD45RA; CD45RO; CD52; CD56; CD62L; CD68; CD80; CD95; CD117; CD127; CD133; CD137 (4-1 BB); CD163; F4/80; IL-4Ra; Sca-1 ; CTLA4; GITR; GARP; LAP; granzyme B; LFA-1 ; transferrin receptor; NKp46, perforin, CD4+; Th1; Th2; Th17; Th40; Th22; Th9; Tfh, Canonical Treg. FoxP3+; Tr1; Th3; Treg17; T_{RE}G; CDCP1, NT5E, EpCAM, CEA, gpA33, Mucins, TAG-72, Carbonic anhydrase IX, PSMA, Folate binding protein, Gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII , GD2, GD3, BCMA, MUC16 (CA125), L1CAM, LeY, MSLN, IL13Rα1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gplOO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6, E7, ETV6-AML, sperm protein 17, XAGE1, Tie 2, MAD-CT-1, MAD-CT-2, Major histocompatibility complex class I-related gene protein (MR1), urokinase-type plasminogen activator receptor (uPAR), Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C) CD49f, CD151 CD340, CD200, tkrA, trkB, or trkC, or an antigenic fragment or antigenic portion thereof.

### 2. ABD Targets An Antigen Characteristic of a T Cell

In some embodiments, the antigen binding domain targets an antigen characteristic of a T cell. In some embodiments, the ABD binds an antigen associated with a T cell. In some instances, such an antigen is expressed by a T cell or is located on the surface of a T cell. In some embodiments, the antigen characteristic of a T cell or the T cell associated antigen is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/ threonine kinase, receptor guanylyl cyclase, histidine kinase associated receptor, AKT1; AKT2; AKT3; ATF2; BCL10; CALM1; CD3D (CD3δ); CD3E (CD3ε); CD3G (CD3y); CD4; CD8; CD28; CD45; CD80 (B7-1); CD86 (B7-2); CD247 (CD3ζ; CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38y); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

### 3. ABD Targets an Antigen Characteristic of an Autoimmune Or Inflammatory Disorder

In some embodiments, the antigen binding domain targets an antigen characteristic of an autoimmune or inflammatory disorder. In some embodiments, the ABD binds an antigen associated with an autoimmune or inflammatory disorder. In some instances, the antigen is expressed by a cell associated with an autoimmune or inflammatory disorder. In some embodiments, the autoimmune or inflammatory disorder is selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave’s disease, autoimmune hemolytic anemia, Hemophilia A, Primary Sjogren’s Syndrome, thrombotic thrombocytopenia purrpura, neuromyelits optica, Evan’s syndrome, IgM mediated neuropathy, cyroglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary nonlimiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/ threonine kinase, receptor guanylyl cyclase, or histidine kinase associated receptor.

In some embodiments, an antigen binding domain of a CAR binds to a ligand expressed on B cells, plasma cells, or plasmablasts. In some embodiments, an antigen binding domain of a CAR binds to CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See US 2003/0077249; WO 2017/058753; WO 2017/058850, the contents of which are herein incorporated by reference.

### 4. ABD Targets An Antigen Characteristic of Senescent Cells

In some embodiments, the antigen binding domain targets an antigen characteristic of senescent cells, e.g., urokinase-type plasminogen activator receptor (uPAR). In some embodiments, the ABD binds an antigen associated with a senescent cell. In some instances, the antigen is expressed by a senescent cell. In some embodiments, the CAR may be used for treatment or prophylaxis of disorders characterized by the aberrant accumulation of senescent cells, e.g., liver and lung fibrosis, atherosclerosis, diabetes and osteoarthritis.

### 5. ABD Targets An Antigen Characteristic of An Infectious Disease

In some embodiments, the antigen binding domain targets an antigen characteristic of an infectious disease. In some embodiments, the ABD binds an antigen associated with an infectious disease. In some instances, the antigen is expressed by a cell affected by an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Eptstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/ threonine kinase, receptor guanylyl cyclase, histidine kinase associated receptor, HIV Env, gp120, or CD4-induced epitope on HIV-1 Env.

### 6. ABD Binds to a Cell Surface Antigen of a Cell

In some embodiments, an antigen binding domain binds to a cell surface antigen of a cell. In some embodiments, a cell surface antigen is characteristic of (e.g., expressed by) a particular or specific cell type. In some embodiments, a cell surface antigen is characteristic of more than one type of cell.

In some embodiments, a CAR antigen binding domain binds a cell surface antigen characteristic of a T cell, such as a cell surface antigen on a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/ threonine kinase, receptor guanylyl cyclase, or histidine kinase associated receptor.

In some embodiments, an antigen binding domain of a CAR binds a T cell receptor. In some embodiments, a T cell receptor may be AKT1; AKT2; AKT3; ATF2; BCL10; CALM1; CD3D (CD3δ); CD3E (CD3ε); CD3G (CD3y); CD4; CD8; CD28; CD45; CD80 (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38y); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

### 7. Transmembrane Domain

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof. antigen binding domain binds

### 8. Signaling Domain or Plurality of Signaling Domains

In some embodiments, a CAR described herein comprises one or at least one signaling domain selected from one or more of B7-⅟CD80; B7-2/CD86; B7-H1/PD-L1; B7-H2; B7-H3; B7-H4; B7-H6; B7-H7; BTLA/CD272; CD28; CTLA4; Gi24/VISTA/B7-H5; ICOS/CD278; PD1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the at least one signaling domain comprises a CD3 zeta domain or an immunoreceptor tyrosine-based activation motif (ITAM), or functional variant thereof. In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

### 9. Domain Which Upon Successful Signaling of the CAR Induces Expression of a Cytokine Gene

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments, a cytokine gene encodes a pro-inflammatory cytokine. In some embodiments, a cytokine gene encodes IL-1, IL-2, IL-9, IL-12, IL-18, TNF, or IFN-gamma, or functional fragment thereof. In some embodiments, a domain which upon successful signaling of the CAR induces expression of a cytokine gene is or comprises a transcription factor or functional domain or fragment thereof. In some embodiments, a domain which upon successful signaling of the CAR induces expression of a cytokine gene is or comprises a transcription factor or functional domain or fragment thereof. In some embodiments, a transcription factor or functional domain or fragment thereof is or comprises a nuclear factor of activated T cells (NFAT), an NF-kB, or functional domain or fragment thereof. See, e.g., Zhang. C. et al., Engineering CAR T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine and serine residues such as but not limited to glycine-serine doublets. In some embodiments, the CAR comprises two or more spacers, e.g., a spacer between the antigen binding domain and the transmembrane domain and a spacer between the transmembrane domain and a signaling domain.

In some embodiments, any one of the cells described herein comprises a nucleic acid encoding a CAR or a first generation CAR. In some embodiments, a first generation CAR comprises an antigen binding domain, a transmembrane domain, and signaling domain. In some embodiments, a signaling domain mediates downstream signaling during T cell activation.

In some embodiments, any one of the cells described herein comprises a nucleic acid encoding a CAR or a second generation CAR. In some embodiments, a second generation CAR comprises an antigen binding domain, a transmembrane domain, and two signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and/or CAR T cell persistence during T cell activation.

In some embodiments, any one of the cells described herein comprises a nucleic acid encoding a CAR or a third generation CAR. In some embodiments, a third generation CAR comprises an antigen binding domain, a transmembrane domain, and at least three signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and or CAR T cell persistence during T cell activation. In some embodiments, a third generation CAR comprises at least two costimulatory domains. In some embodiments, the at least two costimulatory domains are not the same.

In some embodiments, any one of the cells described herein comprises a nucleic acid encoding a CAR or a fourth generation CAR. In some embodiments, a fourth generation CAR comprises an antigen binding domain, a transmembrane domain, and at least two, three, or four signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and or CAR T cell persistence during T cell activation.

### 10. ABD Comprising an Antibody or Antigen-Binding Portion Thereof

In some embodiments, a CAR antigen binding domain is or comprises an antibody or antigen-binding portion thereof. In some embodiments, a CAR antigen binding domain is or comprises an scFv or Fab. In some embodiments, a CAR antigen binding domain comprises an scFv or Fab fragment of a T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD19 antibody; CD20 antibody; CD21 antibody; CD22 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA4 antibody; GITR antibody GARP antibody; LAP antibody; granzyme B antibody; LFA-1 antibody; MR1 antibody; uPAR antibody; or transferrin receptor antibody.

In some embodiments, a CAR comprises a signaling domain which is a costimulatory domain. In some embodiments, a CAR comprises a second costimulatory domain. In some embodiments, a CAR comprises at least two costimulatory domains. In some embodiments, a CAR comprises at least three costimulatory domains. In some embodiments, a CAR comprises a costimulatory domain selected from one or more of CD27, CD28, 4-1BB, CD134/OX40, CD30, CD40, PD1, ICOS, lymphocyte function-associated antigen-1 (LFA-1), CD2, CD7, LIGHT, NKG2C, B7-H3, a ligand that specifically binds with CD83. In some embodiments, if a CAR comprises two or more costimulatory domains, two costimulatory domains are different. In some embodiments, if a CAR comprises two or more costimulatory domains, two costimulatory domains are the same.

In addition to the CARs described herein, various chimeric antigen receptors and nucleotide sequences encoding the same are known in the art and would be suitable for fusosomal delivery and reprogramming of target cells in vivo and in vitro as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

### 11. Bispecific CARs

In certain embodiments, the at least one antigen binding domain is selected from the group consisting of an antibody, an antigen-binding portion thereof, an scFv, and a Fab. In some embodiments, the CAR is a bispecific CAR comprising two antigen binding domains that bind two different antigens. In some embodiments, the at least one antigen binding domain(s) binds to an antigen selected from the group consisting of CD19, CD22, and BCMA. In certain embodiments, the bispecific CAR binds to CD19 and CD22.

In some embodiments, the gene encoding the CAR is carried by a lentiviral vector. In some embodiments, the CAR is selected from the group consisting of a CD19-specific CAR, a CD20-specific CAR, and a CD22-specific CAR. In some embodiments, the CAR is a bispecific CAR. In some embodiments, the bispecific CAR is a CD19/CD20 bispecific CAR. In some embodiments, the bispecific CAR is a CD19/CD22 bispecific CAR.

### C. Therapeutic Cells Derived From T Cells

Provided herein are hypoimmunogenic cells including, but not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells do not include cells from the patient. In some embodiments, one or more of the donor subjects from which the pool of T cells is obtained are different from the patient. In some embodiments, the primary T cells are from a pool of primary T cells from one or more donor subjects that are different than the recipient subject (e.g., the patient administered the cells). The primary T cells can be obtained from 1, 2, 3, 4, 5, 6, 7, 8, 9, 10, 20, 50, 100 or more donor subjects and pooled together. The primary T cells can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the primary T cells are harvested from one or a plurality of individuals, and in some instances, the primary T cells or the pool of primary T cells are cultured in vitro. In some embodiments, primary T cells or a pool of primary T cells are engineered to exogenously express CD47 and cultured in vitro. In some embodiments, primary T cells or a pool of primary T cells are engineered to exogenously express CD24 and cultured in vitro.

In some embodiments, the primary T cells include, but are not limited to, CD3+ T cells, CD4+ T cells, CD8+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells that express CD45RA (TEMRA cells), tissue-resident memory (Trm) cells, virtual memory T cells, innate memory T cells, memory stem cell (Tsc), γδ T cells, and any other subtype of T cells.

In some embodiments, the hypoimmunogenic cells do not activate an immune response in the patient (e.g., recipient upon administration). Provided are methods of treating a disorder comprising repeat dosing of a population of hypoimmunogenic cells to a subject (e.g., recipient) or patient in need thereof. In some embodiments, a population of hypoimmunogenic cells (e.g., hypoimmunogenic primary T cells) is administered at least twice (e.g., 2, 3, 4, 5, or more) to a human patient. In some embodiments, repeat dosing is based on the response to the administration of the hypoimmunogenic cells. In some embodiments, repeat dosing can occur at 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 1 month, 2 months, 3 months, 4, months, 5 months, 6 months, 12 months, 18 months, 24 months, 30 months, 36 months, 48 months, 54 months, or 60 months or more.

In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor. In some embodiments, the T cells described herein such as the engineered or modified T cells comprise reduced expression of cytotoxic T-lymphocyte-associated protein 4 (CTLA4). In other embodiments, the T cells described herein such as the engineered or modified T cells comprise reduced expression of programmed cell death (PD1). In certain embodiments, the T cells described herein such as the engineered or modified T cells comprise reduced expression of CTLA4 and PD1.

### A. Therapeutic Treatment Response in Cancer

In some embodiments, repeat dosing is based on the response to the administration of the in the treatment of cancer. In some embodiments any positive therapeutic indication can be indicative of the response to treatment and can be indicative of benefits for repeat dosing. In some embodiments, repeat dosing occurs when there is at least a 50%, 60%, 70%, 80%, 90%, 95% or more response after administration of hypoimmune T cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells).

In some embodiments the repeat dosing can be employed to administer a lower, the same, or a higher dose of hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells).

In some embodiments, the response is based on changes in tumor size overall. In some embodiments, the response is based on an indication of at least a minimal reduction in the tumor size overall. In some embodiments, a response of tumor size reduction can be employed as an indication for repeat dosing. In some embodiments, a 5%, 10%, 20%, 30%, 40%, 50%, 60%, 70%, 80%, 90%, 95%, 99%, or 100% reduction in overall tumor size after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing.

In some embodiments, the response is based on changes in tumor growth rate. In some embodiments, the response is based on an indication of at least a minimal reduction in tumor growth rate. In some embodiments, a response of a reduction in the tumor growth rate can be employed as an indication for repeat dosing. In some embodiments, a tumor growth rate reduction of at least 5%, 10%, 20%, 30%, 40%, 50%, 60%, 70%, 80%, 90%, 95%, 99%, or 100% after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, a tumor growth rate reduction of at least 1-fold, 2-fold, 3-fold, 4-fold, 5-fold, or 10-fold or more can be employed as an indication for further repeat dosing.

In some embodiments, the response is based on metastasis and/or metastatic progression. In some embodiments, the response is based on an indication of at least a minimal reduction in metastasis and/or metastatic progression. In some embodiments, a response of a reduction in the number of metastases and/or metastatic progression overall can be employed as an indication for repeat dosing. In some embodiments, a reduction in the number of metastases of at least 5%, 10%, 20%, 30%, 40%, 50%, 60%, 70%, 80%, 90%, 95%, 99%, or 100% after HIP cell administration can be employed as an indication for further repeat dosing. In some embodiments, a reduction in the number of metastases of at least 1-fold, 2-fold, 3-fold, 4-fold, 5-fold, or 10-fold or more after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, metastatic progression can be measured by assessing, for example, the presence or number of circulating tumor cells (CTCs) and/or the number of new metastases. In some embodiments, a reduction in the number of new metastases of at least 5%, 10%, 20%, 30%, 40%, 50%, 60%, 70%, 80%, 90%, 95%, 99%, or 100% after HIP cell administration can be employed as an indication for further repeat dosing. In some embodiments, a reduction in the number of new metastases of at least 1-fold, 2-fold, 3-fold, 4-fold, 5-fold, or 10-fold or more can be employed as an indication for further repeat dosing. In some embodiments, a reduction in the number of CTCs by at least 5%, 10%, 20%, 30%, 40%, 50%, 60%, 70%, 80%, 90%, 95%, 99%, or 100% after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, a reduction in the number of CTCs by at least 1-fold, 2-fold, 3-fold, 4-fold, 5-fold, or 10-fold or more can be employed as an indication for further repeat dosing.

In some embodiments, the response is based on T cell activation. In some embodiments, a response of an increase T cell activation can be employed as an indication for repeat dosing. In some embodiments, the response is based on an indication of at least a minimal increase in T cell activation. In some embodiments, an increase in T cell activation of at least 5%, 10%, 20%, 30%, 40%, 50%, 60%, 70%, 80%, 90%, 95%, 99%, or 100% after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, an increase in T cell activation of at least 1-fold, 2-fold, 3-fold, 4-fold, 5-fold, or 10-fold or more after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells)administration can be employed as an indication for further repeat dosing. In some embodiments, T cell activation increases can be determined based on cytokine assays, including for example, IFNγ secretion, which can be measured by methods well known to those of skill in the art, including for example by flow cytometry or ELISA based assays.

In some embodiments, the response is based on T cell persistence. In some embodiments, a response of an increase in T cell persistence can be employed as an indication for repeat dosing. In some embodiments, the response is based on an indication of at least a minimal increase in T cell persistence. In some embodiments, an increase in T cell persistence of at least 5%, 10%, 20%, 30%, 40%, 50%, 60%, 70%, 80%, 90%, 95%, 99%, or 100% after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, an increase in T cell persistence of at least 1-fold, 2-fold, 3-fold, 4-fold, 5-fold, or 10-fold or more after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, T cell persistence can be determined based on the number of T cells present before and after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration. In some embodiments, T cell persistence includes the number of T cells remaining constant before and after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration. In some embodiments, T cell persistence includes the number of T cells increasing before and after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration.

In some embodiments, the response is based on T cell proliferation. In some embodiments, a response of an increase in T cell proliferation can be employed as an indication for repeat dosing. In some embodiments, the response is based on an indication of at least a minimal increase in T cell proliferation. In some embodiments, an increase in T cell proliferation of at least 5%, 10%, 20%, 30%, 40%, 50%, 60%, 70%, 80%, 90%, 95%, 99%, or 100% after HIP cell administration can be employed as an indication for further repeat dosing. In some embodiments, an increase in T cell proliferation of at least 1-fold, 2-fold, 3-fold, 4-fold, 5-fold, or 10-fold or more after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells)administration can be employed as an indication for further repeat dosing. In some embodiments, T cell proliferation can be determined based on the number of T cells present before and after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration. In some embodiments, an increase in T cell proliferation is determined as an increase in the number of T cells before and after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration. In some embodiments, an increase in the number of T cells by at least 5%, 10%, 20%, 30%, 40%, 50%, 60%, 70%, 80%, 90%, 95%, 99%, or 100% after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration indicates an increase T cell proliferation. In some embodiments, an increase in the number of T cells by at least 1-fold, 2-fold, 3-fold, 4-fold, 5-fold, or 10-fold or more after hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration indicates an increase T cell proliferation.

In some embodiments, the response is based on a reduction in use of “other” therapeutics and/or treatments to treat, reduce, ameliorate, diminish, etc., the cancer that the hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) were administered to treat. In some embodiments, the response is based on an indication of at least a minimal reduction in use of “other” therapeutics and/or treatments to treat, reduce, ameliorate, diminish, etc., the cancer that the hypoimmunogenic cells derived from primary T cells or hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) were administered to treat. In some embodiments, the response is based on a reduction in use of “other” therapeutics and/or treatments by number, time period, duration, and/or further dosing of the “other therapeutics. Such “other” therapeutics can include for example, standard of care treatments for a particular cancer indication.

### B. Therapeutic Treatment Response in Non-Cancer Indications

In some embodiments, a response of an increase in engraftment can be employed as an indication for repeat dosing. In some embodiments, the response is based on an indication of at least a minimal increase in engraftment. In some embodiments, an increase in engraftment by at least 5%, 10%, 20%, 30%, 40%, 50%, 60%, 70%, 80%, 90%, 95%, 99%, or 100% after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, an increase in engraftment by at least 1-fold, 2-fold, 3-fold, 4-fold, 5-fold, or 10-fold or more after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, engraftment is maintained for a longer period of time after HIP cell administration. In some embodiments, engraftment is maintained for 1 day, 3 days, 7 days, 2 weeks, 3 weeks, 4 weeks, 1 month, 2 months, 3 months, 4, months, 5 months, 6 months, 12 months, 18 months, 24 months, 30 months, 36 months, 48 months, 54 months, or 60 months or more after HIP cell administration.

In some embodiments, a response based on the persistence of cells (for example, maintaining the number of cells), including grafted cells, can be employed as an indication for repeat dosing. In some embodiments, the response is based on an indication of at least a minimal increase in persistence of cells (for example, maintaining the number of cells), including grafted cells. In some embodiments, an increase in persistence of cells by at least 5%, 10%, 20%, 30%, 40%, 50%, 60%, 70%, 80%, 90%, 95%, 99%, or 100% after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, pancreatic islet cell types, and/or retinal pigmented epithelium (RPE) cells)administration can be employed as an indication for further repeat dosing. In some embodiments, an increase in persistence of cells by at least 1-fold, 2-fold, 3-fold, 4-fold, 5-fold, or 10-fold or more after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, an increase in persistence of cells in measured by maintaining the number of cells, including the number of grafted cells, after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration. In some embodiments, the persistence of cells is maintained for a longer period of time after HIP cell administration. In some embodiments, the number of cells, including the number of grafted cells is maintained for 1 day, 3 days, 7 days, 2 weeks, 3 weeks, 4 weeks, 1 month, 2 months, 3 months, 4, months, 5 months, 6 months, 12 months, 18 months, 24 months, 30 months, 36 months, 48 months, 54 months, or 60 months or more after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration.

In some embodiments, a response based on maintaining the normal function of cells, including the engrafted cells as well as other cells in the subject such as those that are associated with the indication being treated, can be employed as an indication for repeat dosing. In some embodiments, maintaining the normal function of cells by maintaining at least 60%, 70%, 80%, 90%, 95%, 99%, or 100% of the normal function after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, maintaining the normal function of cells after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be determined based on the number of cells that maintain normal function or the percentage of normal function maintained by one or more cells. In some embodiments, the normal function is maintained by least 60%, 70%, 80%, 90%, 95%, 99%, or 100% of the level of the normal function of a wild-type, non-disease, non-indicated affected cell. In some embodiments, the normal function is considered maintained when at least 60%, 70%, 80%, 90%, 95%, 99%, or 100% of the cells being monitored maintain by least 60%, 70%, 80%, 90%, 95%, 99%, or 100% of the level of the normal function of a wild-type, non-disease, non-indicated affected cell. In some embodiments, the normal function or the percentage of normal function is maintained for a longer period of time after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration. In some embodiments, the normal function or the percentage of normal function is maintained for 1 day, 3 days, 7 days, 2 weeks, 3 weeks, 4 weeks, 1 month, 2 months, 3 months, 4, months, 5 months, 6 months, 12 months, 18 months, 24 months, 30 months, 36 months, 48 months, 54 months, or 60 months or more after HIP cell administration.

In some embodiments, the response is based on a reduction in use of “other” therapeutics and/or treatments to treat, reduce, ameliorate, diminish, restore, etc., the disease state or disease indication that the hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells)were administered to treat. In some embodiments, the response is based on an indication of at least a minimal reduction in use of “other” therapeutics and/or treatments to treat, reduce, ameliorate, diminish, etc., the disease state or disease indication that the hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) were administered to treat. In some embodiments, the response is based on a reduction in use of “other” therapeutics and/or treatments by number, time period, duration, and/or further dosing of the “other therapeutics. Such “other” therapeutics can include for example, standard of care treatments for a particular disease state or disease indication.

In some embodiments, the response is based on a reduction in use of “other” therapeutics and/or treatments to treat, reduce, ameliorate, diminish, restore, etc., the disease state or disease indication that the hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) were administered to treat. In some embodiments, a response is based on the restoration of loss of function overall in the body, as associated with the indication being treated, can be employed as an indication for repeat dosing. In some embodiments, the response is based on an indication of at least a minimal restoration of loss of function overall in the body, as associated with the indication being treated. In some embodiments, restoration of loss of function overall in the body by an increase of at least 5%, 10%, 20%, 30%, 40%, 50%, 60%, 70%, 80%, 90%, 95%, 99%, or 100% after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, restoration of loss of function overall in the body by an increase to at least 60%, 70%, 80%, 90%, 95%, 99%, or 100% of normal overall function after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, restoration of loss of function overall in the body by an increase of at least 1-fold, 2-fold, 3-fold, 4-fold, 5-fold, or 10-fold or more after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, the restoration of loss function overall in the body is maintained 1 day, 3 days, 7 days, 2 weeks, 3 weeks, 4 weeks, 1 month, 2 months, 3 months, 4, months, 5 months, 6 months, 12 months, 18 months, 24 months, 30 months, 36 months, 48 months, 54 months, or 60 months or more after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration.

In some embodiments, the response is based on a reduction in use of “other” therapeutics and/or treatments to treat, reduce, ameliorate, diminish, restore, etc., the disease state or disease indication that the hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) were administered to treat. In some embodiments, a response based on the reduction of the disease state associated with the indication being treated can be employed as an indication for repeat dosing. In some embodiments, the response is based on an indication of at least a minimal reduction of the disease state associated with the indication being treated. In some embodiments, reduction of the disease state by at least 5%, 10%, 20%, 30%, 40%, 50%, 60%, 70%, 80%, 90%, 95%, 99%, or 100% after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, reduction of the disease state by at least 60%, 70%, 80%, 90%, 95%, 99%, or 100% of normal overall function after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, reduction of the disease state by at least 1-fold, 2-fold, 3-fold, 4-fold, 5-fold, or 10-fold or more after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration can be employed as an indication for further repeat dosing. In some embodiments, the restoration of loss function overall in the body is maintained 1 day, 3 days, 7 days, 2 weeks, 3 weeks, 4 weeks, 1 month, 2 months, 3 months, 4, months, 5 months, 6 months, 12 months, 18 months, 24 months, 30 months, 36 months, 48 months, 54 months, or 60 months or more after hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells) administration.

### D. Therapeutic Cells Derived From Pluripotent Stem Cells

Provided herein are hypoimmunogenic cells including, cells derived from pluripotent stem cells, that evade immune recognition. In some embodiments, the cells do not activate an immune response in the patient or subject (e.g., recipient upon administration). Provided are methods of treating a disorder comprising repeat dosing of a population of hypoimmunogenic cells to a recipient subject in need thereof, including for example, administration of hypoimmune cells differentiated from hypoimmune induced pluripotent stem cells (wherein such differentiated cells include, for example, but are not limited to T cells, neural/neuronal cells, beta cells, hepatocytes, cardiomyocytes, endothelial cells, thyroid cells, pancreatic islet cell types, and/or retinal pigmented epithelium (RPE) cells).

In some embodiments, the pluripotent stem cell and any cell differentiated from such a pluripotent stem cell is modified to exhibit reduced expression of MHC class I human leukocyte antigens. In other embodiments, the pluripotent stem cell and any cell differentiated from such a pluripotent stem cell is modified to exhibit reduced expression of MHC class II human leukocyte antigens. In some embodiments, the pluripotent stem cell and any cell differentiated from such a pluripotent stem cell is modified to exhibit reduced expression of MHC class I and II human leukocyte antigens. In some embodiments, the pluripotent stem cell and any cell differentiated from such a pluripotent stem cell is modified to exhibit reduced expression of MHC class I and/or II human leukocyte antigens and exhibit increased CD47 expression. In some instances, the cell overexpresses CD47 by harboring one or more CD47 transgenes. In some embodiments, the pluripotent stem cell and any cell differentiated from such a pluripotent stem cell is modified to exhibit reduced expression of MHC class I and/or II human leukocyte antigens and exhibit increased CD24 expression. In some instances, the cell overexpresses CD24 by harboring one or more CD24 transgenes. Such pluripotent stem cells are hypoimmunogenic pluripotent cells. Such differentiated cells are hypoimmunogenic cells.

Any of the pluripotent stem cells described herein can be differentiated into any cells of an organism and tissue. In some embodiments, the cells exhibit reduced expression of MHC class I and/or II human leukocyte antigens. In some instances, expression of MHC class I and/or II human leukocyte antigens is reduced compared to unmodified or wildtype cell of the same cell type. In some embodiments, the cells exhibit increased CD47 or CD24 expression. In some instances, expression of CD47 is increased in cells encompassed by the present technology as compared to unmodified or wildtype cells of the same cell type. Methods for reducing levels of MHC class I and/or II human leukocyte antigens and increasing the expression of CD47 and CD24 are described herein.

In some embodiments, the cells used in the methods described herein evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject’s immune system. In other words, the cells administered in accordance with the methods described herein are not detectable by immune cells of the immune system. In some embodiments, the cells are cloaked and therefore avoid immune rejection.

Methods of determining whether a pluripotent stem cell and any cell differentiated from such a pluripotent stem cell evades immune recognition include, but are not limited to, IFN-y Elispot assays, microglia killing assays, cell engraftment animal models, cytokine release assays, ELISAs, killing assays using bioluminescence imaging or chromium release assay or Xcelligence analysis, mixed-lymphocyte reactions, immunofluorescence analysis, etc.

Therapeutic cells outlined herein are useful to treat a disorder such as, but not limited to, a cancer, a genetic disorder, a chronic infectious disease, an autoimmune disorder, a neurological disorder, and the like.

### E. Hypoimmunogenic Cells

In some embodiments, the present technology disclosed herein is directed to differentiated cells derived from pluripotent stem cells (e.g., pluripotent stem cells and induced pluripotent stem cells (iPSCs)) or primary T cells that overexpress CD24 or CD47, and have reduced expression or lack expression of MHC class I and/or MHC class II human leukocyte antigens and have reduced expression or lack expression of MHC class I and/or MHC class II human leukocyte antigens. In some embodiments, differentiated cells (including hypoimmune T cells and primary T cells) derived from pluripotent stem cells overexpress CD47 and comprise a genomic modification of the B2M gene. In some embodiments, differentiated cells (including hypoimmune T cells and primary T cells) derived from pluripotent stem cells overexpress CD47 and comprise a genomic modification of the CIITA gene. In some embodiments, differentiated cells (including hypoimmune T cells and primary T cells) derived from pluripotent stem cells overexpress CD47 and comprise genomic modifications of the B2M and CIITA genes. In many embodiments, the differentiated cells (including hypoimmune T cells and primary T cells) are B2M^{-/-}, CIITA^{-/-}, CD47tg cells.

In some embodiments, hypoimmune T cells and primary T cells overexpress CD47 and include a genomic modification of the B2M gene. In some embodiments, hypoimmune T cells and primary T cells overexpress CD47 and include a genomic modification of the CIITA gene. In some embodiments, hypoimmune T cells are produced by differentiating induced pluripotent stem cells such as hypoimmunogenic induced pluripotent stem cells. In many embodiments, hypoimmune T cells and primary T cells are B2M^{-/-}, CIITA^{-/-}, CD47tg cells.

Reduction of MHC I and/or MHC II expression can be accomplished, for example, by one or more of the following: (1) targeting the polymorphic HLA alleles (HLA-A, HLA-B, HLA -C) and MHC-II genes directly; (2) removal of B2M, which will prevent surface trafficking of all MHC-I molecules; and/or (3) deletion of components of the MHC enhanceosomes, such as LRC5, RFX-5, RFXANK, RFXAP, IRF1, NF-Y (including NFY-A, NFY-B, NFY-C), and CIITA that are critical for HLA expression.

In certain embodiments, HLA expression is interfered with. In some embodiments, HLA expression is interfered with by targeting individual HLAs (e.g., knocking out expression of HLA-A, HLA-B and/or HLA-C), targeting transcriptional regulators of HLA expression (e.g., knocking out expression of NLRC5, CIITA, RFX5, RFXAP, RFXANK, NFY-A, NFY-B, NFY-C and/or IRF-1), blocking surface trafficking of MHC class I molecules (e.g., knocking out expression of B2M and/or TAP 1), and/or targeting with HLA-Razor (see, e.g., WO2016183041).

In some embodiments, the stem cells disclosed herein do not express one or more human leukocyte antigens (e.g., HLA-A, HLA-B and/or HLA-C) corresponding to MHC-I and/or MHC-II and are thus characterized as being hypoimmunogenic. For example, in some embodiments, the stem cells disclosed herein have been modified such that the stem cell or a differentiated stem cell prepared therefrom do not express or exhibit reduced expression of one or more of the following MHC-I molecules: HLA-A, HLA-B and HLA-C. In some embodiments, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene.

In certain embodiments, gRNAs that allow simultaneous deletion of all MHC class I alleles by targeting a conserved region in the HLA genes are identified as HLA Razors. In some embodiments, the gRNAs are part of a CRISPR system. In some embodiments, the gRNAs are part of a TALEN system. In some embodiments, an HLA Razor targeting an identified conserved region in HLAs is described in WO2016183041. In some embodiments, multiple HLA Razors targeting identified conserved regions are utilized. It is generally understood that any guide that targets a conserved region in HLAs can act as an HLA Razor.

In certain embodiments, differentiated cells derived from pluripotent stem cells overexpress CD24 and comprise a genomic modification of the B2M gene. In some embodiments, differentiated cells derived from pluripotent stem cells overexpress CD24 and comprise a genomic modification of the CIITA gene. In some embodiments, differentiated cells derived from pluripotent stem cells overexpress CD24 and comprise genomic modifications of the B2M and CIITA genes.

In some embodiments, the present technology is directed to primary T cells that overexpress CD24 or CD47, and have reduced expression or lack expression of MHC class I and/or MHC class II human leukocyte antigens and have reduced expression or lack expression of MHC class I and/or MHC class II human leukocyte antigens. The cells outlined herein overexpress CD24 or CD47 and evade immune recognition. In some embodiments, the primary T cells, pluripotent stem cells, and differentiated cells derived from pluripotent stem cells display reduced levels or activity of MHC class I antigens and/or MHC class II antigens. In certain embodiments, primary T cells overexpress CD47 and comprise a genomic modification of the B2M gene. In some embodiments, primary T cells overexpress CD47 and comprise a genomic modification of the CIITA gene. In some embodiments, primary T cells overexpress CD47 and comprise genomic modifications of the B2M and CIITA genes. In some embodiments, primary T cells overexpress CD24 and comprise a genomic modification of the B2M gene. In some embodiments, primary T cells overexpress CD24 and comprise a genomic modification of the CIITA gene. In some embodiments, primary T cells overexpress CD24 and comprise genomic modifications of the B2M and CIITA genes.

Exemplary primary T cells of the present disclosure are selected from the group consisting of cytotoxic T cells, helper T cells, memory T-cells, regulatory T cells, tissue infiltrating lymphocytes, and combinations thereof. In some embodiments, the primary T cells is a modified primary T cell. In some cases, the modified T cell comprise a modification causing the cell to express at least one chimeric antigen receptor that specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof. In other cases, the modified T cell comprise a modification causing the cell to express at least one protein that modulates a biological effect of interest in an adjacent cell, tissue, or organ when the cell is in proximity to the adjacent cell, tissue, or organ. Useful modifications to primary T cells are described in detail in US2016/0348073 and WO2020/018620, the disclosures are incorporated herein in its entirety. Methods provided are useful for inactivation or ablation of MHC class I expression and/or MHC class II expression in cells such as but not limited to pluripotent stem cells and primary T cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of critical immune genes (e.g., by deleting genomic DNA of critical immune genes) in cells. In certain embodiments, genome editing technologies or other gene modulation technologies are used to insert tolerance-inducing factors in human cells, rendering them and the differentiated cells prepared therefrom hypoimmunogenic cells. As such, the hypoimmunogenic cells have reduced or eliminated expression of MHC I and MHC II expression. In some embodiments, the cells are nonimmunogenic (e.g., do not induce an immune response) in a recipient subject.

The genome editing techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

The practice of the some embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

Provided herein are cells comprising a modification of one or more targeted polynucleotide sequences that regulates the expression of MHC I and/or MHC II. In some embodiments, the modification comprising increasing expression of CD47. In some embodiments, the cells comprise an exogenous or recombinant CD47 polypeptide. Also, provided herein are cells comprising a modification of one or more targeted polynucleotide sequences that regulates the expression of MHC I and/or MHC II. In some embodiments, the modification comprising increasing expression of CD24. In some embodiments, the cells comprise an exogenous CD24 or recombinant polypeptide. In some embodiments, the cell also includes a modification to increase expression of one selected from the group consisting of CD200, HLA-G, HLA-E, HLA-C, HLA-E heavy chain, PD-L1, IDO1, CTLA4-Ig, IL-10, IL-35, FASL, Serpinb9, CCl21, and Mfge8. In some embodiments, the cell further comprises a tolerogenic factor (e.g., an immunomodulatory molecule) selected from the group consisting of DUX4, CD200, HLA-G, HLA-E, HLA-C, HLA-E heavy chain, PD-L1, IDO1, CTLA4-Ig, IL-10, IL-35, FASL, Serpinb9, CCl21, and Mfge8.

In some embodiments, the cell comprises a genomic modification of one or more targeted polynucleotide sequences that regulates the expression of MHC I and/or MHC II. In some embodiments, a genetic editing system is used to modify one or more targeted polynucleotide sequences. In some embodiments, the targeted polynucleotide sequence is one or more selected from the group consisting of B2M and CIITA. In some cases, the targeted polynucleotide sequence is NLRC5. In certain embodiments, the genome of the cell has been altered to reduce or delete critical components of HLA expression.

In some embodiments, the present disclosure provides a cell or population thereof comprising a genome in which a gene has been edited to delete a contiguous stretch of genomic DNA, thereby reducing or eliminating surface expression of MHC class I molecules in the cell or population thereof. In some embodiments, the present disclosure provides a cell or population thereof comprising a genome in which a gene has been edited to delete a contiguous stretch of genomic DNA, thereby reducing or eliminating surface expression of MHC class II molecules in the cell or population thereof. In some embodiments, the present disclosure provides a cell or population thereof comprising a genome in which one or more genes has been edited to delete a contiguous stretch of genomic DNA, thereby reducing or eliminating surface expression of MHC class I and II molecules in the cell or population thereof.

In certain embodiments, the expression of MHC I or MHC II is modulated by targeting and deleting a contiguous stretch of genomic DNA thereby reducing or eliminating expression of a target gene selected from the group consisting of B2M and CIITA. In other cases, the target gene is NLRC5.

In some embodiments, the cells and methods described herein include genomically editing human cells to cleave CIITA gene sequences as well as editing the genome of such cells to alter one or more additional target polynucleotide sequences such as, but not limited to, B2M and NLRC5. In some embodiments, the cells and methods described herein include genomically editing human cells to cleave B2M gene sequences as well as editing the genome of such cells to alter one or more additional target polynucleotide sequences such as, but not limited to, CIITA and NLRC5. In some embodiments, the cells and methods described herein include genomically editing human cells to cleave NLRC5 gene sequences as well as editing the genome of such cells to alter one or more additional target polynucleotide sequences such as, but not limited to, B2M and CIITA.

### F. CD47

In some embodiments, the present disclosure provides a cell or population thereof that has been modified to express the tolerogenic factor (e.g., immunomodulatory polypeptide) CD47. In some embodiments, the present disclosure provides a method for altering a cell genome to express CD47. In some embodiments, the stem cell expresses exogenous CD47. In some instances, the cell expresses an expression vector comprising a nucleotide sequence encoding a human CD47 polypeptide. In some embodiments, the hypoimmunogenic cells provided herein are genetically modified to include one or more exogenous polynucleotides inserted into one or more genomic loci of the hypoimmunogenic cell. In some embodiments, the exogenous polynucleotide inserted into one or more genomic loci of the hypoimmunogenic cell encodes for CD47 polypeptides.

CD47 is a leukocyte surface antigen and has a role in cell adhesion and modulation of integrins. It is expressed on the surface of a cell and signals to circulating macrophages not to eat the cell.

In some embodiments, the cell outlined herein comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a nucleotide sequence encoding a CD47 polypeptide having an amino acid sequence as set forth in NCBI Ref. Sequence Nos. NP_001768.1 and NP_942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises a nucleotide sequence for CD47 as set forth in NCBI Ref. Sequence Nos. NM_001777.3 and NM_198793.2.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a CD47 polypeptide having an amino acid sequence as set forth in NCBI Ref. Sequence Nos. NP_001768.1 and NP_942088.1.

In another embodiment, CD47 protein expression is detected using a Western blot of cell lysates probed with antibodies against the CD47 protein. In another embodiment, reverse transcriptase polymerase chain reactions (RT-PCR) are used to confirm the presence of the exogenous CD47 mRNA.

### G. CD24

In some embodiments, the present disclosure provides a cell or population thereof that has been modified to express the tolerogenic factor (e.g., immunomodulatory polypeptide) CD24. In some embodiments, the present disclosure provides a method for altering a cell genome to express CD24. In some embodiments, the stem cell expresses exogenous CD24. In some instances, the cell expresses an expression vector comprising a nucleotide sequence encoding a human CD24 polypeptide. In some embodiments, the hypoimmunogenic cells provided herein are genetically modified to include one or more exogenous polynucleotides inserted into one or more genomic loci of the hypoimmunogenic cell. In some embodiments, the exogenous polynucleotide inserted into one or more genomic loci of the hypoimmunogenic cell encodes for CD24 polypeptides.

CD24 which is also referred to as a heat stable antigen or small-cell lung cancer cluster 4 antigen is a glycosylated glycosylphosphatidylinositol-anchored surface protein (Pirruccello et al., J Immunol, 1986, 136, 3779-3784; Chen et al., Glycobiology, 2017, 57, 800-806). It binds to Siglec-10 on innate immune cells. Recently it has been shown that CD24 via Siglec-10 acts as an innate immune checkpoint (Barkal et al., Nature, 2019, 572, 392-396).

In some embodiments, the cell outlined herein comprises a nucleotide sequence encoding a CD24 polypeptide has at least 95% sequence identity (e.g., 95%, 96%, 97%, 98%, 99%, or more) to an amino acid sequence set forth in NCBI Ref. Nos. NP_001278666.1, NP_001278667.1, NP_001278668.1, and NP _037362.1. In some embodiments, the cell outlined herein comprises a nucleotide sequence encoding a CD24 polypeptide having an amino acid sequence set forth in NCBI Ref. Nos. NP_001278666.1, NP_001278667.1, NP_001278668.1, and NP_037362.1.

In some embodiments, the cell comprises a nucleotide sequence having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_00129737.1, NM_00129738.1, NM_001291739.1, and NM_013230.3. In some embodiments, the cell comprises a nucleotide sequence as set forth in NCBI Ref. Nos. NM_00129737.1, NM_00129738.1, NM_001291739.1, and NM_013230.3.

In another embodiment, CD24 protein expression is detected using a Western blot of cells lysates probed with antibodies against the CD24 protein. In another embodiment, reverse transcriptase polymerase chain reactions (RT-PCR) are used to confirm the presence of the exogenous CD24 mRNA.

### H. Ciita

In some embodiments, the present technology disclosed herein modulates (e.g., reduces or eliminates) the expression of MHC II genes by targeting and modulating (e.g., reducing or eliminating) Class II transactivator (CIITA) expression. In some embodiments, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence of the present technology is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the hypoimmunogenic cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS:5184-36352 of Table 12 of WO2016183041, which is herein incorporated by reference. In some embodiments, the cell has a reduced ability to induce an immune response in a recipient subject.

Assays to test whether the CIITA gene has been inactivated are known and described herein. In one embodiment, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification.

### I. B2M

In certain embodiments, the present technology disclosed herein modulates (e.g., reduces or eliminates) the expression of MHC-I genes by targeting and modulating (e.g., reducing or eliminating) expression of the accessory chain B2M. In some embodiments, the modulation occurs using a CRISPR/Cas system. By modulating (e.g., reducing or deleting) expression of B2M, surface trafficking of MHC-I molecules is blocked and the cell rendered hypoimmunogenic. In some embodiments, the cell has a reduced ability to induce an immune response in a recipient subject.

In some embodiments, the target polynucleotide sequence of the present technology is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC I molecules - HLA-A, HLA-B, and HLA-C.

In some embodiments, the cells described herein comprise gene modifications at the gene locus encoding the B2M protein. In other words, the cells comprise a genetic modification at the B2M locus. In some instances, the nucleotide sequence encoding the B2M protein is set forth in RefSeq. No. NM_004048.4 and Genbank No. AB021288.1. In some instances, the B2M gene locus is described in NCBI Gene ID No. 567. In certain cases, the amino acid sequence of B2M is depicted as NCBI GenBank No. BAA35182.1. Additional descriptions of the B2M protein and gene locus can be found in Uniprot No. P61769, HGNC Ref. No. 914, and OMIM Ref. No. 109700.

In some embodiments, the hypoimmunogenic cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS:81240-85644 of Table 15 of WO2016183041, which is herein incorporated by reference.

Assays to test whether the B2M gene has been inactivated are known and described herein. In one embodiment, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating genetic modification.

### J. Additional Tolerogenic Factors

In certain embodiments, one or more tolerogenic factors can be inserted or reinserted into genome-edited cells to create immune-privileged universal donor cells, such as universal donor stem cells, universal donor T cells, or universal donor cells. In certain embodiments, the hypoimmunogenic cells disclosed herein have been further modified to express one or more tolerogenic factors. Exemplary tolerogenic factors include, without limitation, one or more of DUX4, CD200, HLA-G, HLA-E, HLA-C, HLA-E heavy chain, PD-L1, IDO1, CTLA4-Ig, IL-10, IL-35, FASL, Serpinb9, CCl21, and Mfge8. In some embodiments, the tolerogenic factors are selected from the group consisting of CD200, HLA-G, HLA-E, HLA-C, HLA-E heavy chain, PD-L1, IDO1, CTLA4-Ig, IL-10, IL-35, FASL, Serpinb9, CCl21, and Mfge8. In some embodiments, the tolerogenic factors are selected from the group consisting of DUX4, HLA-C, HLA-E, HLA-F, HLA-G, PD-L1, CTLA-4-Ig, C1-inhibitor, and IL-35. In some embodiments, the tolerogenic factors are selected from the group consisting of HLA-C, HLA-E, HLA-F, HLA-G, PD-L1, CTLA-4-Ig, C1-inhibitor, and IL-35.

In some instances, a gene editing system such as the CRISPR/Cas system is used to facilitate the insertion of tolerogenic factors, such as the tolerogenic factors into a safe harbor locus, such as the AAVS 1 locus, to actively inhibit immune rejection. In some instances, the tolerogenic factors are inserted into a safe harbor locus using an expression vector.

In some embodiments, the present disclosure provides a cell (e.g., a primary T cell and a hypoimmunogenic stem cell and derivative thereof) or population thereof comprising a genome in which the cell genome has been modified to express CD47. In some embodiments, the present disclosure provides a method for altering a cell genome to express CD47. In some embodiments, at least one ribonucleic acid or at least one pair of ribonucleic acids may be utilized to facilitate the insertion of CD47 into a cell line. In some embodiments, the at least one ribonucleic acid or the at least one pair of ribonucleic acids is selected from the group consisting of SEQ ID NOS:200784-231885 of Table 29 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-C. In some embodiments, the present disclosure provides a method for altering a cell genome to express HLA-C. In some embodiments, at least one ribonucleic acid or at least one pair of ribonucleic acids may be utilized to facilitate the insertion of HLA-C into a cell line. In some embodiments, the at least one ribonucleic acid or the at least one pair of ribonucleic acids is selected from the group consisting of SEQ ID NOS:3278-5183 of Table 10 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-E. In some embodiments, the present disclosure provides a method for altering a cell genome to express HLA-E. In some embodiments, at least one ribonucleic acid or at least one pair of ribonucleic acids may be utilized to facilitate the insertion of HLA-E into a cell line. In some embodiments, the at least one ribonucleic acid or the at least one pair of ribonucleic acids is selected from the group consisting of SEQ ID NOS: 189859-193183 of Table 19 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-F. In some embodiments, the present disclosure provides a method for altering a cell genome to express HLA-F. In some embodiments, at least one ribonucleic acid or at least one pair of ribonucleic acids may be utilized to facilitate the insertion of HLA-F into a cell line. In some embodiments, the at least one ribonucleic acid or the at least one pair of ribonucleic acids is selected from the group consisting of SEQ ID NOS: 688808-399754 of Table 45 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-G. In some embodiments, the present disclosure provides a method for altering a cell genome to express HLA-G. In some embodiments, at least one ribonucleic acid or at least one pair of ribonucleic acids may be utilized to facilitate the insertion of HLA-G into a stem cell line. In some embodiments, the at least one ribonucleic acid or the at least one pair of ribonucleic acids is selected from the group consisting of SEQ ID NOS: 188372-189858 of Table 18 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express PD-L1. In some embodiments, the present disclosure provides a method for altering a cell genome to express PD-L1. In some embodiments, at least one ribonucleic acid or at least one pair of ribonucleic acids may be utilized to facilitate the insertion of PD-L1 into a stem cell line. In some embodiments, the at least one ribonucleic acid or the at least one pair of ribonucleic acids is selected from the group consisting of SEQ ID NOS: 193184-200783 of Table 21 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express CTLA4-Ig. In some embodiments, the present disclosure provides a method for altering a cell genome to express CTLA4-Ig. In some embodiments, at least one ribonucleic acid or at least one pair of ribonucleic acids may be utilized to facilitate the insertion of CTLA4-Ig into a stem cell line. In some embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express CI-inhibitor. In some embodiments, the present disclosure provides a method for altering a cell genome to express CI-inhibitor. In some embodiments, at least one ribonucleic acid or at least one pair of ribonucleic acids may be utilized to facilitate the insertion of CI-inhibitor into a stem cell line. In some embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express IL-35. In some embodiments, the present disclosure provides a method for altering a cell genome to express IL-35. In some embodiments, at least one ribonucleic acid or at least one pair of ribonucleic acids may be utilized to facilitate the insertion of IL-35 into a stem cell line. In some embodiments, the at least one ribonucleic acid or the at least one pair of ribonucleic acids is selected from any one disclosed in WO2016183041, including the sequence listing.

In some embodiments, the tolerogenic factors are expressed in a cell using an expression vector. For example, the expression vector for expressing CD47 in a cell comprises a polynucleotide sequence encoding CD47. The expression vector can be an inducible expression vector. The expression vector can be a viral vector, such as but not limited to, a lentiviral vector.

In some embodiments, the present disclosure provides a cell (e.g., a primary T cell and a hypoimmunogenic stem cell and derivative thereof) or population thereof comprising a genome in which the cell genome has been modified to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In some embodiments, the present disclosure provides a method for altering a cell genome to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In some embodiments, at least one ribonucleic acid or at least one pair of ribonucleic acids may be utilized to facilitate the insertion of the selected polypeptide into a stem cell line. In some embodiments, the at least one ribonucleic acid or the at least one pair of ribonucleic acids is selected from any one disclosed in Appendices 1-47 and the sequence listing of WO2016183041, the disclosure is incorporated herein by references.

### K. Methods of Genetic Modifications

In some embodiments, the rare-cutting endonuclease is introduced into a cell containing the target polynucleotide sequence in the form of a nucleic acid encoding a rare-cutting endonuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

The present technology contemplates altering target polynucleotide sequences in any manner which is available to the skilled artisan utilizing a CRISPR/Cas system. Any CRISPR/Cas system that is capable of altering a target polynucleotide sequence in a cell can be used. Such CRISPR-Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism. In such example, the CRISPR/Cas systems can be used to correct the disease associated SNP in a cell by replacing it with a wild-type allele. As another example, a polynucleotide sequence of a target gene which is responsible for entry or proliferation of a pathogen into a cell may be a suitable target for deletion or insertion to disrupt the function of the target gene to prevent the pathogen from entering the cell or proliferating inside the cell.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, a CRISPR/Cas system includes a Cas protein and at least one to two ribonucleic acids that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to Cas1, Cas2, Cas3, Cas4, Cas5, Cas6, Cas7, Cas8 and Cas9. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to, Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019).

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12apolypeptide fused to a tat domain. In some embodiments, the Cas12a protein comprises a Cas12a polypeptide fused to an oligoarginine domain. In some embodiments, the Cas12aprotein comprises a Cas12apolypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods of the present technology contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA sequences useful for CRISPR/Cas-based targeting of genes described herein are provided in Table 1. The sequences can be found in WO2016183041 filed May 9, 2016, the disclosure including the Tables, Appendices, and Sequence Listing is incorporated herein by reference in its entirety.

TABLE 1

Exemplary gRNA sequences useful for targeting genes

| | | |
|---|---|---|
| Gene Name | SEQ ID NO: | WO2016183041 |
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOS:3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOS:5184-36352 | Table 12, Appendix 5 |
| B2M | SEQ ID NOS:81240-85644 | Table 15, Appendix 8 |
| NLRC5 | SEQ ID NOS:36353-81239 | Table 14, Appendix 7 |
| CD47 | SEQ ID NOS:200784-231885 | Table 29, Appendix 22 |
| HLA-E | SEQ ID NOS:189859-193183 | Table 19, Appendix 12 |
| HLA-F | SEQ ID NOS:688808-699754 | Table 45, Appendix 38 |
| HLA-G | SEQ ID NOS:188372-189858 | Table 18, Appendix 11 |
| PD-L1 | SEQ ID NOS:193184-200783 | Table 21, Appendix 14 |

In some embodiments, the cells of the present technology are made using Transcription Activator-Like Effector Nucleases (TALEN) methodologies.

By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-1. In a particular embodiment, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues co-ordinated with zinc along with the two cysteine residues of a single beta turn (see, e.g., Berg & Shi, Science 271:1081-1085 (1996)).

In some embodiments, the cells are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease may for example correspond to a LAGLIDADG endonuclease, to a HNH endonuclease, or to a GIY-YIG endonuclease. Preferred homing endonuclease can be an I-CreI variant.

In some embodiments, the cells are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the cells are made using RNA silencing or RNA interference (RNAi) to knockdown (e.g., decrease, eliminate, or inhibit) the expression of a polypeptide such as a tolerogenic factor. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, CIITA can be knocked down in a pluripotent stem cell by introducing a CIITA siRNA or transducing a CIITA shRNA-expressing virus into the cell. In some embodiments, RNA interference is employed to reduce or inhibit the expression of at least one selected from the group consisting of CIITA, B2M, and NLRC5.

### L. Overexpression of Tolerogenic Factors

For all of these technologies, well known recombinant techniques are used, to generate recombinant nucleic acids as outlined herein. In certain embodiments, the recombinant nucleic acids encoding a tolerogenic factor may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector’s copy number, the ability to control that copy number, or the expression of any other protein encoded by the vector, such as antibiotic markers.

Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al., Nature 273: 113-120 (1978)). The immediate early promoter of the human cytomegalovirus is conveniently obtained as a HindIII E restriction fragment (Greenaway et al., Gene 18: 355-360 (1982)). The foregoing references are incorporated by reference in their entirety.

The process of introducing the polynucleotides described herein into cells can be achieved by any suitable technique. Suitable techniques include calcium phosphate or lipid-mediated transfection, electroporation, and transduction or infection using a viral vector. In some embodiments, the polynucleotides are introduced into a cell via viral transduction (e.g., lentiviral transduction).

Once altered, the presence of expression of any of the molecule described herein can be assayed using known techniques, such as Western blots, ELISA assays, FACS assays, and the like.

In some embodiments, the present technology provides hypoimmunogenic pluripotent cells that comprise a “suicide gene” or “suicide switch”. These are incorporated to function as a “safety switch” that can cause the death of the hypoimmunogenic pluripotent cells should they grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al., Mol. Therap. 20(10): 1932-1943 (2012), Xu et al., Cell Res. 8:73-8 (1998), both incorporated herein by reference in their entirety.)

In other embodiments, the suicide gene is an inducible Caspase protein. An inducible Caspase protein comprises at least a portion of a Caspase protein capable of inducing apoptosis. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, AP1903. Thus, the suicide function of iCasp9 is triggered by the administration of a chemical inducer of dimerization (CID). I n some embodiments, the CID is the small molecule drug API 903. Dimerization causes the rapid induction of apoptosis. (See WO2011146862; Stasi et al., N. Engl. J. Med 365;18 (2011); Tey et al., Biol. Blood Marrow Transplant. 13:913-924 (2007), each of which are incorporated by reference herein in their entirety.)

### M. Generation of Hypoimmunogenic Pluripotent Stem Cells

The present technology provides methods of producing hypoimmunogenic pluripotent cells. In some embodiments, the method comprises generating pluripotent stem cells. The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct 3 / 4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al., World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen. In general, these reprogramming factor genes are provided on episomal vectors such as are known in the art and commercially available.

In general, as is known in the art, iPSCs are made from non-pluripotent cells such as, but not limited to, blood cells, fibroblasts, etc., by transiently expressing the reprogramming factors as described herein.

### N. Assays For Hypoimmunogenicity Phenotypes and Retention of Pluripotency

Once the hypoimmunogenic cells have been generated, they may be assayed for their hypoimmunogenicity and/or retention of pluripotency as is described in WO2016183041 and WO2018132783.

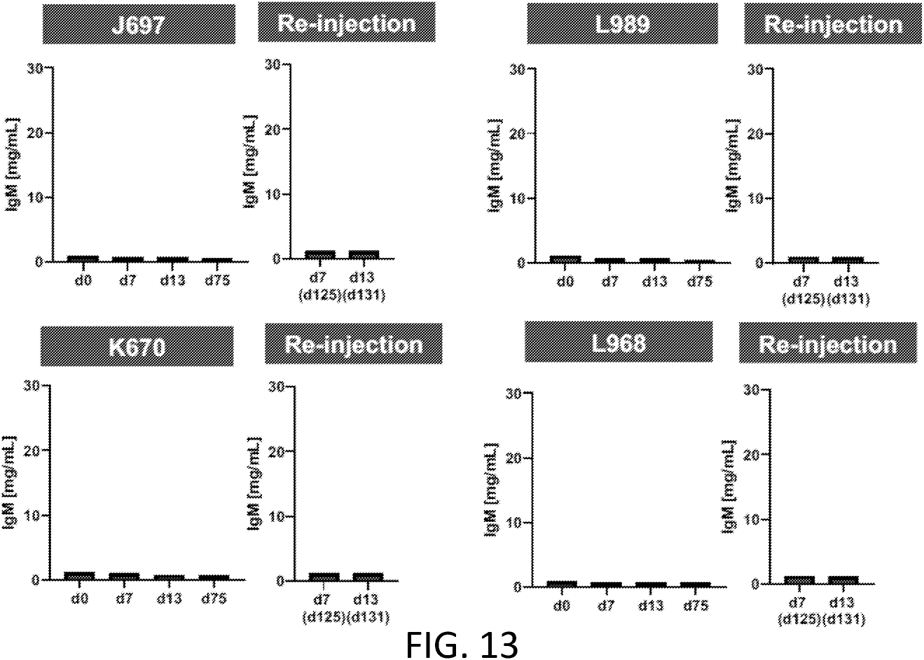

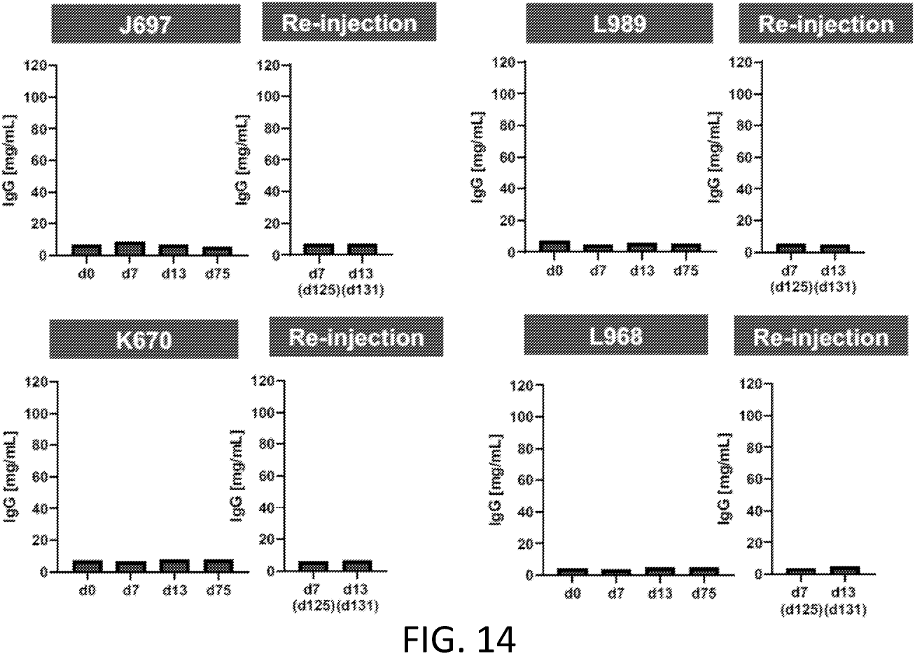

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g. teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In one embodiment, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, B, C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of HLA I and II (or MHC I and II), the hypoimmunogenic cells of the technology have a reduced susceptibility to macrophage phagocytosis and NK cell killing. The resulting hypoimmunogenic cells “escape” the immune macrophage and innate pathways due to the expression of one or more CD24 transgenes.

### O. Maintenance of Hypoimmunogenic Pluripotent Stem Cells

Once the hypoimmunogenic pluripotent stem cells have been generated, they can be maintained an undifferentiated state as is known for maintaining iPSCs. For example, the cells can be cultured on Matrigel using culture media that prevents differentiation and maintains pluripotency. In addition, they can be in culture medium under conditions to maintain pluripotency.

### P. Administration of Hypoimmunogenic Cells Differentiated From Hypoimmunogenic Pluripotent Cells

The present technology provides HIP cells that are differentiated into different cell types for subsequent transplantation into recipient subjects. Differentiation can be assayed as is known in the art, generally by evaluating the presence of cell-specific markers. As will be appreciated by those in the art, the differentiated hypoimmunogenic pluripotent cell derivatives can be transplanted using techniques known in the art that depends on both the cell type and the ultimate use of these cells. In some embodiments, T lymphocytes (T cells) are derived from the hypoimmunogenic induced pluripotent stem (HIP) cells described herein. In some embodiments, the T cells derived from HIP cells are administered as a mixture of CD4+ and CD8+ cells. In some embodiments, the T cells derived from HIP cells that are administered are CD4+ cells. In some embodiments the T cells derived from HIP cells that are administered are CD8+ cells. In some embodiments, the T cells derived from HIP cells are administered as non-activated T cells.

### 1. Cardiac Cells Differentiated From Hypoimmunogenic Pluripotent Cells

The present technology provides hypoimmunogenic pluripotent cells that are differentiated into different cardiac cell types for subsequent transplantation or engraftment into subjects (e.g., recipients). As will be appreciated by those in the art, the methods for differentiation depend on the desired cell type using known techniques. Exemplary cardiac cell types include, but are not limited to, a cardiomyocyte, nodal cardiomyocyte, conducting cardiomyocyte, working cardiomyocyte, cardiomyocyte precursor cell, cardiac stem cell, atrial cardiac stem cell, ventricular cardiac stem cell, epicardial cell, hematopoietic cell, vascular endothelial cell, endocardial endothelial cell, cardiac valve interstitial cell, cardiac pacemaker cell, and the like.

In some embodiments, the cardiomyocyte precursor includes a cell that is capable (without dedifferentiation or reprogramming) of giving rise to progeny that include mature (end-stage) cardiomyocytes. Cardiomyocyte precursor cells can often be identified using one or more markers selected from GATA-4, Nkx2.5, and the MEF-2 family of transcription factors. In some instances, cardiomyocytes refer to immature cardiomyocytes or mature cardiomyocytes that express one or more markers (sometimes at least 3 or 5 markers) from the following list: cardiac troponin I (cTnl), cardiac troponin T (cTnT), sarcomeric myosin heavy chain (MHC), GATA-4, Nkx2.5, N-cadherin, β2- adrenoceptor (bI-AII), ANF, the MEF-2 family of transcription factors, creatine kinase MB (CK-MB), myoglobin, or atrial natriuretic factor (ANF). In some embodiments, the cardiac cells demonstrate spontaneous periodic contractile activity. In some cases, when that cardiac cells are cultured in a suitable tissue culture environment with an appropriate Ca²⁺ concentration and electrolyte balance, the cells can be observed to contract in a periodic fashion across one axis of the cell, and then release from contraction, without having to add any additional components to the culture medium. In some embodiments, the cardiac cells are hypoimmunogenic cardiac cells.

In some embodiments, cardiac cells described herein are administered to a recipient subject to treat a cardiac disorder selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, and autoimmune endocarditis.

Accordingly, provided herein are methods for the treatment and prevention of a cardiac injury or a cardiac disease or disorder in a subject in need thereof. The methods described herein can be used to treat, ameliorate, prevent or slow the progression of a number of cardiac diseases or their symptoms, such as those resulting in pathological damage to the structure and/or function of the heart. The terms “cardiac disease,” “cardiac disorder,” and “cardiac injury,” are used interchangeably herein and refer to a condition and/or disorder relating to the heart, including the valves, endothelium, infarcted zones, or other components or structures of the heart. Such cardiac diseases or cardiac-related disease include, but are not limited to, myocardial infarction, heart failure, cardiomyopathy, congenital heart defect, heart valve disease or dysfunction, endocarditis, rheumatic fever, mitral valve prolapse, infective endocarditis, hypertrophic cardiomyopathy, dilated cardiomyopathy, myocarditis, cardiomegaly, and/or mitral insufficiency, among others.

In some embodiments, the method of producing a population of hypoimmunogenic cardiac cells from a population of hypoimmunogenic pluripotent (HIP) cells by in vitro differentiation comprises: (a) culturing a population of HIP cells in a culture medium comprising a GSK inhibitor; (b) culturing the population of HIP cells in a culture medium comprising a WNT antagonist to produce a population of pre-cardiac cells; and (c) culturing the population of pre-cardiac cells in a culture medium comprising insulin to produce a population of hypoimmune cardiac cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the WNT antagonist is IWR1, a derivative thereof, or a variant thereof. In some instances, the WNT antagonist is at a concentration ranging from about 2 mM to about 10 mM.

In some embodiments, the population of hypoimmunogenic cardiac cells is isolated from non-cardiac cells. In some embodiments, the isolated population of hypoimmunogenic cardiac cells are expanded prior to administration. In certain embodiments, the isolated population of hypoimmunogenic cardiac cells are expanded and cryopreserved prior to administration.

Other useful methods for differentiating induced or embryonic pluripotent stem cells into cardiac cells are described, for example, in US2017/0152485; US2017/0058263; US2017/0002325; US2016/0362661; US2016/0068814;, US9,062,289;US7,897,389; and US7,452,718. Additional methods for producing cardiac cells from induced or embryonic pluripotent stem cells are described in, for example, Xu et al., Stem Cells and Development, 2006, 15(5): 631-9, Burridge et al., Cell Stem Cell, 2012, 10: 16-28, and Chen et al., Stem Cell Res, 2015, 15(2):365-375.

In various embodiments, hypoimmunogenic cardiac cells can be cultured in culture medium comprising a BMP pathway inhibitor, a WNT signaling activator, a WNT signaling inhibitor, a WNT agonist, a WNT antagonist, a Src inhibitor, a EGFR inhibitor, a PCK activator, a cytokine, a growth factor, a cardiotropic agent, a compound, and the like.

The WNT signaling activator includes, but is not limited to, CHIR99021. The PCK activator includes, but is not limited to, PMA. The WNT signaling inhibitor includes, but is not limited to, a compound selected from KY02111, SO3031 (KY01-I), SO2031 (KY02-I), and SO3042 (KY03-I), and XAV939. The Src inhibitor includes, but is not limited to, A419259. The EGFR inhibitor includes, but is not limited to, AG1478.

Non-limiting examples of an agent for generating a cardiac cell from an iPSC include activin A, BMP -4, Wnt3a, VEGF, soluble frizzled protein, cyclosporin A, angiotensin II, phenylephrine, ascorbic acid, dimethylsulfoxide, 5-aza-2′-deoxycytidine, and the like.

The cells can be cultured on a surface, such as a synthetic surface to support and/or promote differentiation of hypoimmunogenic pluripotent cells into cardiac cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethylene glycol) dimethacrylate, 1,6-hexanediol propoxylate diacrylate, neopentyl glycol diacrylate, trimethylolpropane benzoate diacrylate, trimethylolpropane eihoxylate (1 EO/QH) methyl, tricyclo[5.2.1.0^{2,6}] decane dimethanol diacrylate, neopentyl glycol ethoxylate diacrylate, and trimethylolpropane triacrylate. Acrylate synthesized as known in the art or obtained from a commercial vendor, such as Polysciences, Inc., Sigma Aldrich, Inc. and Sartomer, Inc.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

The efficacy of cardiac cells prepared as described herein can be assessed in animal models for cardiac cryoinjury, which causes 55% of the left ventricular wall tissue to become scar tissue without treatment (Li et al., Ann. Thorac. Surg. 62:654, 1996; Sakai et al., Ann. Thorac. Surg. 8:2074, 1999, Sakai et al., Thorac. Cardiovasc. Surg. 118:715, 1999). Successful treatment can reduce the area of the scar, limit scar expansion, and improve heart function as determined by systolic, diastolic, and developed pressure. Cardiac injury can also be modeled using an embolization coil in the distal portion of the left anterior descending artery (Watanabe et al., Cell Transplant. 7:239, 1998), and efficacy of treatment can be evaluated by histology and cardiac function.

In some embodiments, the administration comprises implantation into the subject’s heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the patient administered the engineered cardiac cells is also administered a cardiac drug. Illustrative examples of cardiac drugs that are suitable for use in combination therapy include, but are not limited to, growth factors, polynucleotides encoding growth factors, angiogenic agents, calcium channel blockers, antihypertensive agents, antimitotic agents, inotropic agents, anti-atherogenic agents, anti-coagulants, beta- blockers, anti-arhythmic agents, anti-inflammatory agents, vasodilators, thrombolytic agents, cardiac glycosides, antibiotics, antiviral agents, antifungal agents, agents that inhibit protozoans, nitrates, angiotensin converting enzyme (ACE) inhibitors, angiotensin II receptor antagonist, brain natriuretic peptide (BNP); antineoplastic agents, steroids, and the like.

The effects of therapy according to the methods can be monitored in a variety of ways. For instance, an electrocardiogram (ECG) or holier monitor can be utilized to determine the efficacy of treatment. An ECG is a measure of the heart rhythms and electrical impulses, and is a very effective and non-invasive way to determine if therapy has improved or maintained, prevented, or slowed degradation of the electrical conduction in a subject’s heart. The use of a holier monitor, a portable ECG that can be worn for long periods of time to monitor heart abnormalities, arrhythmia disorders, and the like, is also a reliable method to assess the effectiveness of therapy. An ECG or nuclear study can be used to determine improvement in ventricular function.

### 2. Neural Cells Differentiated From Hypoimmunogenic Pluripotent Cells

The present technology provides hypoimmunogenic pluripotent cells that are differentiated into different neural cell types for subsequent transplantation or engraftment into recipient subjects. As will be appreciated by those in the art, the methods for differentiation depend on the desired cell type using known techniques. Exemplary neural cell types include, but are not limited to, cerebral endothelial cells, neurons (e.g., dopaminergic neurons), glial cells, and the like.

In some embodiments, differentiation of induced pluripotent stem cells is performed by exposing or contacting cells to specific factors which are known to produce a specific cell lineage(s), so as to target their differentiation to a specific, desired lineage and/or cell type of interest. In some embodiments, terminally differentiated cells display specialized phenotypic characteristics or features. In many embodiments, the stem cells described herein are differentiated into a neuroectodermal, neuronal, neuroendocrine, dopaminergic, cholinergic, serotonergic (5-HT), glutamatergic, GABAergic, adrenergic, noradrenergic, sympathetic neuronal, parasympathetic neuronal, sympathetic peripheral neuronal, or glial cell population. In some instances, the glial cell population includes a microglial (e.g., amoeboid, ramified, activated phagocytic, and activated non-phagocytic) cell population or a macroglial (central nervous system cell: astrocyte, oligodendrocyte, ependymal cell, and radial glia; and peripheral nervous system cell: Schwann cell and satellite cell) cell population, or the precursors and progenitors of any of the preceding cells.

Protocols for generating different types of neural cells are described in PCT Application No. WO2010144696, U.S. Pat. Nos. 9,057,053; 9,376,664; and 10,233,422. Additional descriptions of methods for differentiating hypoimmunogenic pluripotent cells can be found, for example, in Deuse et al., Nature Biotechnology, 2019, 37, 252-258 and Han et al., Proc Natl Acad Sci USA, 2019, 116(21), 10441-10446. Methods for determining the effect of neural cell transplantation in an animal model of a neurological disorder or condition are described in the following references: for spinal cord injury - Curtis et al., Cell Stem Cell, 2018, 22, 941-950; for Parkinson’s disease - Kikuchi et al., Nature, 2017, 548:592-596; for ALS - Izrael et al., Stem Cell Research, 2018, 9(1): 152 and Izrael et al., IntechOpen, DOI: 10.5772/intechopen.72862; for epilepsy - Upadhya et al., PNAS, 2019, 116(1):287-296.

In some embodiments, neural cells are administered to a subject to treat Parkinson’s disease, Huntington disease, multiple sclerosis, other neurodegenerative disease or condition, attention deficit hyperactivity disorder (ADHD), Tourette Syndrome (TS), schizophrenia, psychosis, depression, other neuropsychiatric disorder. In some embodiments, neural cells described herein are administered to a subject to treat or ameliorate stroke. In some embodiments, the neurons and glial cells are administered to a subject with amyotrophic lateral sclerosis (ALS). In some embodiments, cerebral endothelial cells are administered to alleviate the symptoms or effects of cerebral hemorrhage. In some embodiments, dopaminergic neurons are administered to a patient with Parkinson’s disease. In some embodiments, noradrenergic neurons, GABAergic interneurons are administered to a patient who has experienced an epileptic seizure. In some embodiments, motor neurons, interneurons, Schwann cells, oligodendrocytes, and microglia are administered to a patient who has experienced a spinal cord injury.

In some embodiments, cerebral endothelial cells (ECs), precursors, and progenitors thereof are differentiated from pluripotent stem cells (e.g., induced pluripotent stem cells) on a surface by culturing the cells in a medium comprising one or more factors that promote the generation of cerebral ECs or neural cell. In some instances, the medium includes one or more of the following: CHIR-99021, VEGF, basic FGF, and Y-27632. In some embodiments, the medium includes a supplement designed to promote survival and functionality for neural cells.

### I. Cerebral Endothelial Cells

In some embodiments, cerebral endothelial cells (ECs), precursors, and progenitors thereof are differentiated from pluripotent stem cells on a surface by culturing the cells in an unconditioned or conditioned medium. In some instances, the medium comprises factors or small molecules that promote or facilitate differentiation. In some embodiments, the medium comprises one or more factors or small molecules selected from the group consisting of VEGR, FGF, SDF-1, CHIR-99021, Y-27632, SB 431542, and any combination thereof. In some embodiments, the surface for differentiation comprises one or more extracellular matrix proteins. The surface can be coated with the one or more extracellular matrix proteins. The cells can be differentiated in suspension and then put into a gel matrix form, such as matrigel, gelatin, or fibrin/thrombin forms to facilitate cell survival. In some cases, differentiation is assayed as is known in the art, generally by evaluating the presence of cell-specific markers.

In some embodiments, the cerebral endothelial cells express or secrete a factor selected from the group consisting of CD31, VE cadherin, and a combination thereof. In certain embodiments, the cerebral endothelial cells express or secrete one or more of the factors selected from the group consisting of CD31, CD34, CD45, CD117 (c-kit), CD146, CXCR4, VEGF, SDF-1, PDGF, GLUT-1, PECAM-1, eNOS, claudin-5, occludin, ZO-1, p-glycoprotein, von Willebrand factor, VE-cadherin, low density lipoprotein receptor LDLR, low density lipoprotein receptor-related protein 1 LRP1, insulin receptor INSR, leptin receptor LEPR, basal cell adhesion molecule BCAM, transferrin receptor TFRC, advanced glycation endproduct-specific receptor AGER, receptor for retinol uptake STRA6, large neutral amino acids transporter small subunit 1 SLC7A5, excitatory amino acid transporter 3 SLC1A1, sodium-coupled neutral amino acid transporter 5 SLC38A5, solute carrier family 16 member 1 SLC16A1, ATP-dependent translocase ABCB1, ATP- ABCC2binding cassette transporter ABCG2, multidrug resistance-associated protein 1 ABCC1, canalicular multispecific organic anion transporter 1 ABCC2, multidrug resistance-associated protein 4 ABCC4, and multidrug resistance-associated protein 5 ABCC5.

In some embodiments, the cerebral ECs are characterized with one or more of the features selected from the group consisting of high expression of tight junctions, high electrical resistance, low fenestration, small perivascular space, high prevalence of insulin and transferrin receptors, and high number of mitochondria.

In some embodiments, cerebral ECs are selected or purified using a positive selection strategy. In some instances, the cerebral ECs are sorted against an endothelial cell marker such as, but not limited to, CD31. In other words, CD31 positive cerebral ECs are isolated. In some embodiments, cerebral ECs are selected or purified using a negative selection strategy. In some embodiments, undifferentiated or pluripotent stem cells are removed by selecting for cells that express a pluripotency marker including, but not limited to, TRA-1-60 and SSEA-1.

### II. Dopaminergic Neurons

In some embodiments, hypoimmunogenic induced pluripotent stem (HIP) cells described herein are differentiated into dopaminergic neurons include neuronal stem cells, neuronal progenitor cells, immature dopaminergic neurons, and mature dopaminergic neurons.

In some cases, the term “dopaminergic neurons” includes neuronal cells which express tyrosine hydroxylase (TH), the rate-limiting enzyme for dopamine synthesis. In some embodiments, dopaminergic neurons secrete the neurotransmitter dopamine, and have little or no expression of dopamine hydroxylase. A dopaminergic (DA) neuron can express one or more of the following markers: neuron-specific enolase (NSE), 1-aromatic amino acid decarboxylase, vesicular monoamine transporter 2, dopamine transporter, Nurr-1, and dopamine-2 receptor (D2 receptor). In certain cases, the term “neural stem cells” includes a population of pluripotent cells that have partially differentiated along a neural cell pathway and express one or more neural markers including, for example, nestin. Neural stem cells may differentiate into neurons or glial cells (e.g., astrocytes and oligodendrocytes). The term “neural progenitor cells” includes cultured cells which express FOXA2 and low levels of b-tubulin, but not tyrosine hydroxylase. Such neural progenitor cells have the capacity to differentiate into a variety of neuronal subtypes; particularly a variety of dopaminergic neuronal subtypes, upon culturing the appropriate factors, such as those described herein.

In some embodiments, the DA neurons derived from hypoimmunogenic induced pluripotent stem (HIP) cells are administered to a patient, e.g., human patient to treat a neurodegenerative disease or condition. In some cases, the neurodegenerative disease or condition is selected from the group consisting of Parkinson’s disease, Huntington disease, and multiple sclerosis. In other embodiments, the DA neurons are used to treat or ameliorate one or more symptoms of a neuropsychiatric disorder, such as attention deficit hyperactivity disorder (ADHD), Tourette Syndrome (TS), schizophrenia, psychosis, and depression. In yet other embodiments, the DA neurons are used to treat a patient with impaired DA neurons.

To characterize and monitor DA differentiation and assess the DA phenotype, expression of any number of molecular and genetic markers can be evaluated. For example, the presence of genetic markers can be determined by various methods known to those skilled in the art. Expression of molecular markers can be determined by quantifying methods such as, but not limited to, qPCR-based assays, immunoassays, immunocytochemistry assays, immunoblotting assays, and the like. Exemplary markers for DA neurons include, but are not limited to, TH, b-tubulin, paired box protein (Pax6), insulin gene enhancer protein (Isl1), nestin, diaminobenzidine (DAB), G protein-activated inward rectifier potassium channel 2 (GIRK2), microtubule-associated protein 2 (MAP-2), NURR1, dopamine transporter (DAT), forkhead box protein A2 (FOXA2), FOX3, doublecortin, and LIM homeobox transcription factor 1-beta (LMX1B), and the like. In some embodiments, the DA neurons express one or more of the markers selected from corin, FOXA2, TuJ1, NURR1, and any combination thereof.

In some embodiments, DA neurons are assessed according to cell electrophysiological activity. The electrophysiology of the cells can be evaluated by using assays knowns to those skilled in the art. For instance, whole-cell and perforated patch clamp, assays for detecting electrophysiological activity of cells, assays for measuring the magnitude and duration of action potential of cells, and functional assays for detecting dopamine production of DA cells.

In some embodiments, DA neuron differentiation is characterized by spontaneous rhythmic action potentials, and high-frequency action potentials with spike frequency adaption upon injection of depolarizing current. In other embodiments, DA differentiation is characterized by the production of dopamine. The level of dopamine produced is calculated by measuring the width of an action potential at the point at which it has reached half of its maximum amplitude (spike half-maximal width).

In some embodiments, the differentiated DA neurons are transplanted either intravenously or by injection at particular locations in the patient. In some embodiments, the differentiated DA cells are transplanted into the substantia nigra (particularly in or adjacent of the compact region), the ventral tegmental area (VTA), the caudate, the putamen, the nucleus accumbens, the subthalamic nucleus, or any combination thereof, of the brain to replace the DA neurons whose degeneration resulted in Parkinson’s disease. The differentiated DA cells can be injected into the target area as a cell suspension. Alternatively, the differentiated DA cells can be embedded in a support matrix or scaffold when contained in such a delivery device. In some embodiments, the scaffold is biodegradable. In other embodiments, the scaffold is not biodegradable. The scaffold can comprise natural or synthetic (artificial) materials.

The delivery of the DA neurons can be achieved by using a suitable vehicle such as, but not limited to, liposomes, microparticles, or microcapsules. In other embodiments, the differentiated DA neurons are administered in a pharmaceutical composition comprising an isotonic excipient. The pharmaceutical composition is prepared under conditions that are sufficiently sterile for human administration. In some embodiments, the DA neurons differentiated from HIP cells are supplied in the form of a pharmaceutical composition. General principles of therapeutic formulations of cell compositions are found in Cell Therapy: Stem Cell Transplantation, Gene Therapy, and Cellular Immunotherapy, G. Morstyn & W. Sheridan eds, Cambridge University Press, 1996, and Hematopoietic Stem Cell Therapy, E. Ball, J. Lister & P. Law, Churchill Livingstone, 2000, the disclosures are incorporated herein by reference.

In addition to DA neurons, other neuronal cells, precursors, and progenitors thereof can be differentiated from the HIP cells outlined herein by culturing the cells in medium comprising one or more factors or additive. Non-limiting examples of factors and additives include GDNF, BDNF, GM-CSF, B27, basic FGF, basic EGF, NGF, CNTF, SMAD inhibitor, Wnt antagonist, SHH signaling activator, and any combination thereof. In some embodiments, the SMAD inhibitor is selected from the group consisting of SB431542, LDN-193189, Noggin PD169316, SB203580, LY364947, A77-01, A-83-01, BMP4, GW788388, GW6604, SB-505124, lerdelimumab, metelimumab, GC-I008, AP-12009, AP-110I4, LY550410, LY580276, LY364947, LY2109761, SB-505124, E-616452 (RepSox ALK inhibitor), SD-208, SMI6, NPC-30345, K 26894, SB-203580, SD-093, activin-M108A, P144, soluble TBR2-Fc, DMH-1, dorsomorphin dihydrochloride and derivatives thereof. In some embodiments, the Wnt antagonist is selected from the group consisting of XAV939, DKK1, DKK-2, DKK-3, Dkk-4, SFRP-1, SFRP-2, SFRP-5, SFRP-3, SFRP-4, WIF-1, Soggy, IWP-2, IWR1, ICG-001, KY0211, Wnt-059, LGK974, IWP-L6 and derivatives thereof. In some embodiments, the SHH signaling activator is selected from the group consisting of Smoothened agonist (SAG), SAG analog, SHH, C25-SHH, C24-SHH, purmorphamine, Hg--Ag and derivatives thereof.

In some embodiments, the neurons expression one or more of the markers selected from the group consisting of glutamate ionotropic receptor NMDA type subunit 1 GRIN1, glutamate decarboxylase 1 GAD1, gamma-aminobutyric acid GABA, tyrosine hydroxylase TH, LIM homeobox transcription factor 1-alpha LMX1A, Forkhead box protein O1 FOXO1, Forkhead box protein A2 FOXA2, Forkhead box protein O4 FOXO4, FOXG1, 2′,3′-cyclic-nucleotide 3′-phosphodiesterase CNP, myelin basic protein MBP, tubulin beta chain 3 TUB3, tubulin beta chain 3 NEUN, solute carrier family 1 member 6 SLC1A6, SST, PV, calbindin, RAX, LHX6, LHX8, DLX1, DLX2, DLX5, DLX6, SOX6, MAFB, NPAS1, ASCL1, SIX6, OLIG2, NKX2.1, NKX2.2, NKX6.2, VGLUT1, MAP2, CTIP2, SATB2, TBR1, DLX2, ASCL1, ChAT, NGFI-B, c-fos, CRF, RAX, POMC, hypocretin, NADPH, NGF, Ach, VAChT, PAX6, EMX2p75, CORIN, TUJ1, NURR1, and any combination thereof. In some embodiments, the dopaminergic neurons express one or more of the markers selected from CORIN, FOXA2, TUJ1, NURR1, and any combination thereof.

In some embodiments, stem cells described herein are differentiated into dopaminergic neurons include dopaminergic progenitors. The stem cells are cultured in a differentiation medium comprising a supplement or additive to induce neuronal differentiation. In some embodiments, the cells are cultured in the presence of a supplement or additive to induce floor plate cells. In some embodiments, the supplement or additive includes BMP inhibitor LDN193189, ALK-5 inhibitor A83-01, Smoothened agonist purmorphamine, FGF8, GSK3 inhibitor CHIR99021, glial cell line-derived neurotrophic factor, GDNF, ascorbic acid, brain-derived neurotrophic factor BDNF, dibutyryladenosine cyclic monophosphate dbcAMP, ROCK inhibitor Y-27632, and the like.

In some embodiments, the method of producing a population of hypoimmunogenic dopaminergic neurons from a population of hypoimmunogenic induced pluripotent stem cells (HIP cells) by in vitro differentiation comprises (a) culturing the population of HIP cells in a first culture medium comprising one or more factors selected from the group consisting of sonic hedgehog (SHH), BDNF, EGF, bFGF, FGF8, WNT1, retinoic acid, a GSK3 inhibitor, an ALK inhibitor, and a ROCK inhibitor to produce a population of immature dopaminergic neurons; and (b) culturing the population of immature dopaminergic neurons in a second culture medium that is different than the first culture medium to produce a population of dopaminergic neurons. In some embodiments, the GSK inhibitor is CHIR-99021, a derivative thereof, or a variant thereof. In some instances, the GSK inhibitor is at a concentration ranging from about 2 mM to about 10 pM. In some embodiments, the ALK inhibitor is SB-431542, a derivative thereof, or a variant thereof. In some instances, the ALK inhibitor is at a concentration ranging from about 1 mM to about 10 mM. In some embodiments, the first culture medium and/or second culture medium are absent of animal serum.

In some embodiments, the population of hypoimmunogenic dopaminergic neurons is isolated from non-neuronal cells. In some embodiments, the isolated population of hypoimmunogenic dopaminergic neurons are expanded prior to administration. In certain embodiments, the isolated population of hypoimmunogenic dopaminergic neurons are expanded and cryopreserved prior to administration.

Methods for differentiating pluripotent stem cells are described in, e.g., Kikuchi et al., Nature, 2017, 548, 592-596; Kriks et al., Nature, 2011, 547-551; Doi et al., Stem Cell Reports, 2014, 2, 337-50; Perrier et al., Proc Natl Acad Sci USA, 2004, 101, 12543-12548; Chambers et al., Nat Biotechnol, 2009, 27, 275-280; and Kirkeby et al., Cell Reports, 2012, 1, 703-714.

Useful descriptions of neurons derived from stem cells and methods of making thereof can be found, for example, in Kirkeby et al., Cell Rep, 2012, 1:703-714; Kriks et al., Nature, 2011, 480:547-551; Wang et al., Stem Cell Reports, 2018, 11(1):171-182; Lorenz Studer, “Chapter 8 - Strategies for Bringing Stem Cell-Derived Dopamine Neurons to the clinic-The NYSTEM Trial” in Progress in Brain Research, 2017, volume 230, pg. 191-212; Liu et al., Nat Protoc, 2013, 8:1670-1679; Upadhya et al., Curr Protoc Stem Cell Biol, 38, 2D.7.1-2D.7.47; U.S. Publication Appl. No. 20160115448, and US8,252,586; US8,273,570; US9,487,752 and US10,093,897, the contents are incorporated herein by reference in their entirety.

In some embodiments, glial cells including microglia, astrocytes, oligodendrocytes, ependymal cells and Schwann cells, glial precursors, and glial progenitors thereof are produced by differentiating pluripotent stem cells into therapeutically effective glial cells and the like. Differentiation of hypoimmunogenic pluripotent stem cells produces hypoimmunogenic neural cells, such as hypoimmunogenic glial cells.

In some embodiments, glial cells, precursors, and progenitors thereof generated by culturing pluripotent stem cells in medium comprising one or more agents selected from the group consisting of retinoic acid, IL-34, M-CSF, FLT3 ligand, GM-CSF, CCL2, a TGFbeta inhibitor, a BMP signaling inhibitor, a SHH signaling activator, FGF, platelet derived growth factor PDGF, PDGFR-alpha, HGF, IGF-1, noggin, sonic hedgehog (SHH), dorsomorphin, noggin, and any combination thereof. In certain instances, the BMP signaling inhibitor is LDN193189, SB431542, or a combination thereof. In some embodiments, the glial cells express NKX2.2, PAX6, SOX10, brain derived neurotrophic factor BDNF, neutrotrophin-3 NT-3, NT-4, epidermal growth factor EGF, ciliary neurotrophic factor CNTF, nerve growth factor NGF, FGF8, EGFR, OLIG1, OLIG2, myelin basic protein MBP, GAP-43, LNGFR, nestin, GFAP, CD11b, CD11c, CX3CR1, P2RY12, IBA-1, TMEM119, CD45, and any combination thereof. Exemplary differentiation medium can include any specific factors and/or small molecules that may facilitate or enable the generation of a glial cell type as recognized by those skilled in the art.

To determine if the cells generated according to the in vitro differentiation protocol display glial cell characteristics and features, the cells can be transplanted into an animal model. In some embodiments, the glial cells are injected into an immunocompromised mouse, e.g., an immunocompromised shiverer mouse. The glial cells are administered to the brain of the mouse and after a pre-selected amount of time the engrafted cells are evaluated. In some instances, the engrafted cells in the brain are visualized by using immunostaining and imaging methods. In some embodiments, it is determined that the glial cells express known glial cell biomarkers.

Useful methods for generating glial cells, precursors, and progenitors thereof from stem cells are found, for example, in US7,579,188; US7,595,194; US8,263,402; US8,206,699; US8,252,586; US9,193,951; US9,862,925; US8,227,247; US9,709,553; US2018/0187148; US2017/0198255; US2017/0183627; US2017/0182097; US2017/253856; US2018/0236004; WO2017/172976; and WO2018/093681.

In some embodiments, differentiation of pluripotent stem cells is performed by exposing or contacting cells to specific factors which are known to produce a specific cell lineage(s), so as to target their differentiation to a specific, desired lineage and/or cell type of interest. In some embodiments, terminally differentiated cells display specialized phenotypic characteristics or features. In certain embodiments, the stem cells described herein are differentiated into a neuroectodermal, neuronal, neuroendocrine, dopaminergic, cholinergic, serotonergic (5-HT), glutamatergic, GABAergic, adrenergic, noradrenergic, sympathetic neuronal, parasympathetic neuronal, sympathetic peripheral neuronal, or glial cell population. In some instances, the glial cell population includes a microglial (e.g., amoeboid, ramified, activated phagocytic, and activated non-phagocytic) cell population or a macroglial (central nervous system cell: astrocyte, oligodendrocyte, ependymal cell, and radial glia; and peripheral nervous system cell: Schwann cell and satellite cell) cell population, or the precursors and progenitors of any of the preceding cells.

Protocols for generating different types of neural cells are described in PCT Application No. WO2010144696, U.S Pat. Nos. 9,057,053; 9,376,664; and 10,233,422. Additional descriptions of methods for differentiating hypoimmunogenic pluripotent cells can be found, for example, in Deuse et al., Nature Biotechnology, 2019, 37, 252-258 and Han et al., Proc Natl Acad Sci USA, 2019, 116(21), 10441-10446. Methods for determining the effect of neural cell transplantation in an animal model of a neurological disorder or condition are described in the following references: for spinal cord injury - Curtis et al., Cell Stem Cell, 2018, 22, 941-950; for Parkinson’s disease - Kikuchi et al., Nature, 2017, 548:592-596; for ALS - Izrael et al., Stem Cell Research, 2018, 9(1):152 and Izrael et al., IntechOpen, DOI: 10.5772/intechopen.72862; for epilepsy - Upadhya et al., PNAS, 2019, 116(1):287-296

The efficacy of neural cell transplants for spinal cord injury can be assessed in, for example, a rat model for acutely injured spinal cord, as described by McDonald, et al., Nat. Med., 1999, 5:1410) and Kim, et al., Nature, 2002, 418:50. For instance, successful transplants may show transplant-derived cells present in the lesion 2-5 weeks later, differentiated into astrocytes, oligodendrocytes, and/or neurons, and migrating along the spinal cord from the lesioned end, and an improvement in gait, coordination, and weight-bearing. Specific animal models are selected based on the neural cell type and neurological disease or condition to be treated.

The neural cells can be administered in a manner that permits them to engraft to the intended tissue site and reconstitute or regenerate the functionally deficient area. For instance, neural cells can be transplanted directly into parenchymal or intrathecal sites of the central nervous system, according to the disease being treated. In some embodiments, any of the neural cells described herein including cerebral endothelial cells, neurons, dopaminergic neurons, ependymal cells, astrocytes, microglial cells, oligodendrocytes, and Schwann cells are injected into a patient by way of intravenous, intraspinal, intracerebroventricular, intrathecal, intra-arterial, intramuscular, intraperitoneal, subcutaneous, intramuscular, intra-abdominal, intraocular, retrobulbar and combinations thereof. In some embodiments, the cells are injected or deposited in the form of a bolus injection or continuous infusion. In certain embodiments, the neural cells are administered by injection into the brain, apposite the brain, and combinations thereof. The injection can be made, for example, through a burr hole made in the subject’s skull. Suitable sites for administration of the neural cell to the brain include, but are not limited to, the cerebral ventricle, lateral ventricles, cisterna magna, putamen, nucleus basalis, hippocampus cortex, striatum, caudate regions of the brain and combinations thereof.

Additional descriptions of neural cells including dopaminergic neurons for use in the present technology are found in WO2020/018615, the disclosure is herein incorporated by reference in its entirety.

### 3. Endothelial Cells Differentiated From Hypoimmunogenic Pluripotent Cells

The present technology provides hypoimmunogenic pluripotent cells that are differentiated into various endothelial cell types for subsequent transplantation or engraftment into subjects (e.g., recipients). As will be appreciated by those in the art, the methods for differentiation depend on the desired cell type using known techniques. Exemplary endothelial cell types include, but are not limited to, a capillary endothelial cell, vascular endothelial cell, aortic endothelial cell, arterial endothelial cell, venous endothelial cell, renal endothelial cell, brain endothelial cell, liver endothelial cell, and the like.

The endothelial cells outlined herein can express one or more endothelial cell markers. Non-limiting examples of such markers include VE-cadherin (CD 144), ACE (angiotensin-converting enzyme) (CD 143), BNH9/BNF13, CD31, CD34, CD54 (ICAM-1), CD62E (E-Selectin), CD105 (Endoglin), CD146, Endocan (ESM-1), Endoglyx-1, Endomucin, Eotaxin-3, EPAS1 (Endothelial PAS domain protein 1), Factor VIII related antigen, FLI-1, Flk-1 (KDR, VEGFR-2), FLT-1 (VEGFR-1), GATA2, GBP-1 (guanylate- binding protein-1), GRO-alpha, HEX, ICAM-2 (intercellular adhesion molecule 2), LM02, LYVE-1, MRB (magic roundabout), Nucleolin, PAL-E (pathologische anatomie Leiden- endothelium), RTKs, sVCAM-1, TALI, TEM1 (Tumor endothelial marker 1), TEM5 (Tumor endothelial marker 5), TEM7 (Tumor endothelial marker 7), Thrombomodulin (TM, CD141), VCAM-1 (vascular cell adhesion molecule- 1) (CD106), VEGF (Vascular endothelial growth factor), vWF (von Willebrand factor), ZO-1, endothelial cell-selective adhesion molecule (ESAM), CD102, CD93, CD184, CD304, and DLL4.

In some embodiments, the endothelial cells are genetically modified to express an exogenous gene encoding a protein of interest such as but not limited to an enzyme, hormone, receptor, ligand, or drug that is useful for treating a disorder/condition or ameliorating symptoms of the disorder/condition. Standard methods for genetically modifying endothelial cells are described, e.g., in US5,674,722.

Such endothelial cells can be used to provide constitutive synthesis and delivery of polypeptides or proteins, which are useful in prevention or treatment of disease. In this way, the polypeptide is secreted directly into the bloodstream or other area of the body (e.g., central nervous system) of the individual. In some embodiments, the endothelial cells can be modified to secrete insulin, a blood clotting factor (e.g., Factor VIII or von Willebrand Factor), alpha-1 antitrypsin, adenosine deaminase, tissue plasminogen activator, interleukins (e.g., IL-1, IL-2, IL-3), and the like.

In certain embodiments, the endothelial cells can be modified in a way that improves their performance in the context of an implanted graft. Non-limiting illustrative examples include secretion or expression of a thrombolytic agent to prevent intraluminal clot formation, secretion of an inhibitor of smooth muscle proliferation to prevent luminal stenosis due to smooth muscle hypertrophy, and expression and/or secretion of an endothelial cell mitogen or autocrine factor to stimulate endothelial cell proliferation and improve the extent or duration of the endothelial cell lining of the graft lumen.

In some embodiments, the engineered endothelial cells are utilized for delivery of therapeutic levels of a secreted product to a specific organ or limb. For example, a vascular implant lined with endothelial cells engineered (transduced) in vitro can be grafted into a specific organ or limb. The secreted product of the transduced endothelial cells will be delivered in high concentrations to the perfused tissue, thereby achieving a desired effect to a targeted anatomical location.

In other embodiments, the endothelial cells are genetically modified to contain a gene that disrupts or inhibits angiogenesis when expressed by endothelial cells in a vascularizing tumor. In some cases, the endothelial cells can also be genetically modified to express any one of the selectable suicide genes described herein which allows for negative selection of grafted endothelial cells upon completion of tumor treatment.

In some embodiments, endothelial cells described herein are administered to a recipient subject to treat a vascular disorder selected from the group consisting of vascular injury, cardiovascular disease, vascular disease, peripheral vascular disease, ischemic disease, myocardial infarction, congestive heart failure, peripheral vascular obstructive disease, hypertension, ischemic tissue injury, reperfusion injury, limb ischemia, stroke, neuropathy (e.g., peripheral neuropathy or diabetic neuropathy), organ failure (e.g., liver failure, kidney failure, and the like), diabetes, rheumatoid arthritis, osteoporosis, cerebrovascular disease, hypertension, angina pectoris and myocardial infarction due to coronary artery disease, renal vascular hypertension, renal failure due to renal artery stenosis, claudication of the lower extremities, other vascular condition or disease.

In some embodiments, the hypoimmunogenic pluripotent cells are differentiated into endothelial colony forming cells (ECFCs) to form new blood vessels to address peripheral arterial disease. Techniques to differentiate endothelial cells are known. See, e.g., Prasain et al., doi: 10.1038/nbt.3048, incorporated herein by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the method of producing a population of hypoimmunogenic endothelial cells from a population of hypoimmunogenic pluripotent cells by in vitro differentiation comprises: (a) culturing a population of HIP cells in a first culture medium comprising a GSK inhibitor; (b) culturing the population of HIP cells in a second culture medium comprising VEGF and bFGF to produce a population of pre-endothelial cells; and (c) culturing the population of pre-endothelial cells in a third culture medium comprising a ROCK inhibitor and an ALK inhibitor to produce a population of hypoimmunogenic endothelial cells.

In some embodiments, the GSK inhibitor is CHIR-99021, a derivative thereof, or a variant thereof. In some instances, the GSK inhibitor is at a concentration ranging from about 1 mM to about 10 mM. In some embodiments, the ROCK inhibitor is Y-27632, a derivative thereof, or a variant thereof. In some instances, the ROCK inhibitor is at a concentration ranging from about 1 pM to about 20 pM. In some embodiments, the ALK inhibitor is SB-431542, a derivative thereof, or a variant thereof. In some instances, the ALK inhibitor is at a concentration ranging from about 0.5 pM to about 10 pM.

In some embodiments, the first culture medium comprises from 2 pM to about 10 pM of CHIR-99021. In some embodiments, the second culture medium comprises 50 ng/ml VEGF and 10 ng/ml bFGF. In other embodiments, the second culture medium further comprises Y-27632 and SB-431542. In various embodiments, the third culture medium comprises 10 pM Y-27632 and 1 pM SB-431542. In certain embodiments, the third culture medium further comprises VEGF and bFGF. In particular instances, the first culture medium and/or the second medium is absent of insulin.

The cells can be cultured on a surface, such as a synthetic surface to support and/or promote differentiation of hypoimmunogenic pluripotent cells into cardiac cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethyiene glycol) dimethacrylate, 1,6-hexanediol propoxylate diacrylate, neopentyl glycol diacrylate, trimethylolpropane benzoate diacrylate, trimethylolpropane eihoxylate (1 EO/QH) methyl, tricyclo[5.2.1.0^{2,6}] decane dimethanol diacrylate, neopentyl glycol ethoxylate diacrylate, and trimethylolpropane triacrylate. Acrylate synthesized as known in the art or obtained from a commercial vendor, such as Polysciences, Inc., Sigma Aldrich, Inc. and Sartomer, Inc.

In some embodiments, the endothelial cells may be seeded onto a polymer matrix. In some cases, the polymer matrix is biodegradable. Suitable biodegradable matrices are well known in the art and include collagen-GAG, collagen, fibrin, PLA, PGA, and PLA/PGA copolymers. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides.

Non-biodegradable polymers may also be used as well. Other non- biodegradable, yet biocompatible polymers include polypyrrole, polyanibnes, polythiophene, polystyrene, polyesters, non-biodegradable polyurethanes, polyureas, poly(ethylene vinyl acetate), polypropylene, polymethacrylate, polyethylene, polycarbonates, and poly(ethylene oxide). The polymer matrix may be formed in any shape, for example, as particles, a sponge, a tube, a sphere, a strand, a coiled strand, a capillary network, a film, a fiber, a mesh, or a sheet. The polymer matrix can be modified to include natural or synthetic extracellular matrix materials and factors.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

In some embodiments, the population of hypoimmunogenic endothelial cells is isolated from non-endothelial cells. In some embodiments, the isolated population of hypoimmunogenic endothelial cells are expanded prior to administration. In certain embodiments, the isolated population of hypoimmunogenic endothelial cells are expanded and cryopreserved prior to administration.

Additional descriptions of endothelial cells for use in the present technology are found in WO2020/018615, the disclosure is herein incorporated by reference in its entirety.

### 4. Thyroid Cells Differentiated From Hypoimmunogenic Pluripotent Cells

In some embodiments, the hypoimmunogenic pluripotent cells are differentiated into thyroid progenitor cells and thyroid follicular organoids that can secrete thyroid hormones to address autoimmune thyroiditis. Techniques to differentiate thyroid cells are known the art. See, e.g. Kurmann et al., Cell Stem Cell, 2015 Nov 5;17(5):527-42, incorporated herein by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

### 5. Hepatocytes Differentiated From Hypoimmunogenic Pluripotent Cells

In some embodiments, the hypoimmunogenic pluripotent cells are differentiated into hepatocytes to address loss of the hepatocyte functioning or cirrhosis of the liver. There are a number of techniques that can be used to differentiate HIP cells into hepatocytes; see, for example Pettinato et al., doi: 10.1038/spre32888, Snykers et al., Methods Mol Biol 698:305-314 (2011), Si-Tayeb et al., Hepatology 51:297-305 (2010) and Asgari et al., Stem Cell Rev 9:493- 504 (2013), all of which are incorporated herein by reference in their entirety and specifically for the methodologies and reagents for differentiation. Differentiation is assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release and glycogen storage.

### 6. Pancreatic Islet Cells Differentiated From Hypoimmunogenic Pluripotent Cells

The present technology provides hypoimmunogenic pluripotent cells that are differentiated into various pancreatic islet cell types for subsequent transplantation or engraftment into subjects (e.g., recipients). As will be appreciated by those in the art, the methods for differentiation depend on the desired cell type using known techniques. Exemplary pancreatic islet cell types include, but are not limited to, pancreatic islet progenitor cell, immature pancreatic islet cell, mature pancreatic islet cell, and the like. In some embodiments, pancreatic cells described herein are administered to a subject to treat diabetes.

In some embodiments, pancreatic islet cells are derived from the hypoimmunogenic pluripotent cells described herein. Useful method for differentiating pluripotent stem cells into pancreatic islet cells are described, for example, in US9,683,215; US9,157,062; and US8,927,280.

In some embodiments, the pancreatic islet cells produced by the methods as disclosed herein secretes insulin. In some embodiments, a pancreatic islet cell exhibits at least two characteristics of an endogenous pancreatic islet cell, for example, but not limited to, secretion of insulin in response to glucose, and expression of beta cell markers.

Exemplary beta cell markers or beta cell progenitor markers include, but are not limited to, c-peptide, Pdxl, glucose transporter 2 (Glut2), HNF6, VEGF, glucokinase (GCK), prohormone convertase (PC ⅓), Cdcpl, NeuroD, Ngn3, Nkx2.2, Nkx6.1, Nkx6.2, Pax4, Pax6, Ptfla, Isll, Sox9, Soxl7, and FoxA2.

In some embodiments, the isolated pancreatic islet cells produce insulin in response to an increase in glucose. In various embodiments, the isolated pancreatic islet cells secrete insulin in response to an increase in glucose. In some embodiments, the cells have a distinct morphology such as a cobblestone cell morphology and/or a diameter of about 17 pm to about 25 pm.

In some embodiments, the hypoimmunogenic pluripotent cells are differentiated into beta-like cells or islet organoids for transplantation to address type I diabetes mellitus (T1DM). Cell systems are a promising way to address T1DM, see, e.g., Ellis et al. (Nat Rev Gastroenterol Hepatol., 14(10):612-628 (2017), incorporated herein by reference. Additionally, Pagliuca et al. (Cell, 159(2):428-39 (2014)) reports on the successful differentiation of β-cells from hiPSCs, the contents incorporated herein by reference in its entirety and in particular for the methods and reagents outlined there for the large-scale production of functional human β cells from human pluripotent stem cells). Furthermore, Vegas et al. shows the production of human β cells from human pluripotent stem cells followed by encapsulation to avoid immune rejection by the host; Vegas et al., Nat Med, 2016, 22(3):306-11, incorporated herein by reference in its entirety and in particular for the methods and reagents outlined there for the large-scale production of functional human β cells from human pluripotent stem cells.

In some embodiments, the method of producing a population of hypoimmunogenic pancreatic islet cells from a population of hypoimmunogenic pluripotent cells by in vitro differentiation comprises: (a) culturing the population of HIP cells in a first culture medium comprising one or more factors selected from the group consisting insulin-like growth factor (IGF), transforming growth factor (TGF), fibroblast growth factor (EGF), epidermal growth factor (EGF), hepatocyte growth factor (HGF), sonic hedgehog (SHH), and vascular endothelial growth factor (VEGF), transforming growth factor-b (TORb) superfamily, bone morphogenic protein-2 (BMP2), bone morphogenic protein-7 (BMP7), a GSK inhibitor, an ALK inhibitor, a BMP type 1 receptor inhibitor, and retinoic acid to produce a population of immature pancreatic islet cells; and (b) culturing the population of immature pancreatic islet cells in a second culture medium that is different than the first culture medium to produce a population of hypoimmune pancreatic islet cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the ALK inhibitor is SB-431542, a derivative thereof, or a variant thereof. In some instances, the ALK inhibitor is at a concentration ranging from about 1 pM to about 10 pM. In some embodiments, the first culture medium and/or second culture medium are absent of animal serum.

In some embodiments, the population of hypoimmunogenic pancreatic islet cells is isolated from non-pancreatic islet cells. In some embodiments, the isolated population of hypoimmunogenic pancreatic islet cells are expanded prior to administration. In certain embodiments, the isolated population of hypoimmunogenic pancreatic islet cells are expanded and cryopreserved prior to administration.

Differentiation is assayed as is known in the art, generally by evaluating the presence of β cell associated or specific markers, including but not limited to, insulin. Differentiation can also be measured functionally, such as measuring glucose metabolism, see generally Muraro et al., Cell Syst. 3(4): 385-394.e3 (2016), hereby incorporated by reference in its entirety, and specifically for the biomarkers outlined there. Once the beta cells are generated, they can be transplanted (either as a cell suspension or within a gel matrix as discussed herein) into the portal vein/liver, the omentum, the gastrointestinal mucosa, the bone marrow, a muscle, or subcutaneous pouches.

Additional descriptions of pancreatic islet cells including dopaminergic neurons for use are found in WO2020/018615, the disclosure is herein incorporated by reference in its entirety.

### 7. Retinal Pigmented Epithelium (RPE) Cells Differentiated From Hypoimmunogenic Pluripotent Cells

The present technology provides hypoimmunogenic pluripotent cells that are differentiated into various RPE cell types for subsequent transplantation or engraftment into subjects (e.g., recipients). As will be appreciated by those in the art, the methods for differentiation depend on the desired cell type using known techniques. Exemplary RPE cell types include, but are not limited to, retinal pigmented epithelium (RPE) cell, RPE progenitor cell, immature RPE cell, mature RPE cell, functional RPE cell, and the like.

Useful methods for differentiating embryonic pluripotent stem cells into RPE cells are described in, for example, US9,458,428 and US9,850,463, the disclosures are herein incorporated by reference in their entirety, including the specifications. Additional methods for producing RPE cells from human embryonic or induced pluripotent stem cells can be found in, for example, Lamba et al., PNAS, 2006, 103(34): 12769-12774; Mellough et al., Stem Cells, 2012, 30(4):673-686; Idelson et al., Cell Stem Cell, 2009, 5(4): 396-408; Rowland et al., Journal of Cellular Physiology, 2012, 227(2):457-466, Buchholz et al., Stem Cells Trans Med, 2013, 2(5): 384-393, and da Cruz et al., Nat Biotech, 2018, 36:328-337.

In some embodiments, RPE cells described herein are administered to a subject to treat an eye disorder selected from the group consisting of wet macular degeneration, dry macular degeneration, juvenile macular degeneration (e.g., Stargardt disease, Best disease, and juvenile retinoschisis), Leber’s Congenital Ameurosis, retinitis pigmentosa, retinal detachment, age-related macular degeneration (AMD), early AMD, intermediate AMD, late AMD, non-neovascular age-related macular degeneration, and the like.

Human pluripotent stem cells have been differentiated into RPE cells using the techniques outlined in Kamao et al., Stem Cell Reports 2014:2:205-18, hereby incorporated by reference in its entirety and in particular for the methods and reagents outlined there for the differentiation techniques and reagents; see also Mandai et al., N Engl J Med, 2017, 376:1038-1046, the contents herein incorporated in its entirety for techniques for generating sheets of RPE cells and transplantation into patients. Differentiation can be assayed as is known in the art, generally by evaluating the presence of RPE associated and/or specific markers or by measuring functionally. See for example Kamao et al., Stem Cell Reports, 2014, 2(2):205-18, the contents incorporated herein by reference in its entirety and specifically for the markers outlined in the first paragraph of the results section.

In some embodiments, the method of producing a population of hypoimmunogenic retinal pigmented epithelium (RPE) cells from a population of hypoimmunogenic pluripotent cells by in vitro differentiation comprises: (a) culturing the population of hypoimmunogenic pluripotent cells in a first culture medium comprising any one of the factors selected from the group consisting of activin A, bFGF, BMP4/7, DKK1, IGF1, noggin, a BMP inhibitor, an ALK inhibitor, a ROCK inhibitor, and a VEGFR inhibitor to produce a population of pre-RPE cells; and (b) culturing the population of pre-RPE cells in a second culture medium that is different than the first culture medium to produce a population of hypoimmunogenic RPE cells. In some embodiments, the ALK inhibitor is SB-431542, a derivative thereof, or a variant thereof. In some instances, the ALK inhibitor is at a concentration ranging from about 2 mM to about 10 pM. In some embodiments, the ROCK inhibitor is Y-27632, a derivative thereof, or a variant thereof. In some instances, the ROCK inhibitor is at a concentration ranging from about 1 pM to about 10 pM. In some embodiments, the first culture medium and/or second culture medium are absent of animal serum.

Differentiation can be assayed as is known in the art, generally by evaluating the presence of RPE associated and/or specific markers or by measuring functionally. See for example Kamao et al., Stem Cell Reports, 2014, 2(2):205-18, the contents are herein incorporated by reference in its entirety and specifically for the results section.

Additional descriptions of RPE cells for use are found in WO2020/018615, the disclosure is herein incorporated by reference in its entirety.

For therapeutic application, cells prepared according to the disclosed methods can typically be supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

### 8. T Lymphocytes

Provided herein, T lymphocytes (T cells) are derived from the hypoimmunogenic induced pluripotent stem (HIP) cells described. Methods for generating T cells, including CAR T cells, from pluripotent stem cells (e.g., iPSCs) are described, for example, in Iriguchi et al., Nature Communications 12, 430 (2021); Themeli et al., Cell Stem Cell, 16(4):357-366 (2015); Themeli et al., Nature Biotechnology 31:928-933 (2013).

In some embodiments, the hypoimmunogenic induced pluripotent stem cell-derived T cell includes a chimeric antigen receptor (CAR). Any suitable CAR can be included in the hypoimmunogenic induced pluripotent stem cell-derived T cell, including the CARs described herein. In some embodiments, the hypoimmunogenic induced pluripotent stem cell-derived T cell includes a polynucleotide encoding a CAR. Any suitable method can be used to insert the CAR into a genomic locus of the hypoimmunogenic cell including the gene editing methods described herein (e.g., a CRISPR/Cas system).

HIP-derived T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

### IV. EXAMPLES

### Example 1: Effects of B2M-/-CIITA-/- CD47 Transgenic Human Induced Pluripotent Stem Cells Transplanted into Non-Human Primates

To study the effect of decreasing MHC I and MHC II expression and increasing CD47 expression, human B2M^{-/-}CIITA^{-/-} CD47 transgenic induced pluripotent stem cells (HIP cells) were transplanted into rhesus monkey (non-human primate or NHP) recipients (xenogeneic transplantation).

Study design and administration. Eight NHPs (F/M, 2-3 kg, 12-36 months of age) were randomized into two groups (n = 4) for blinded administration of either wild-type or HIP cells. Under an IACUC-approved protocol, each NHP was administered four subcutaneous injections of ~10⁷ human wild-type or HIP cells into the back. Blood was drawn for analysis prior to injection (day 0) at days 7 and 13 following injection. Both the wild-type and HIP cells also transgenically expressed firefly luciferase for bioluminescence imaging (BLI), and cell survival was monitored by BLI.

No systemic immune responses were observed in the NHPs receiving xenogeneic HIP cells following the initial injection, in contrast to the NHPs injected with wild-type cells. However, the cells did not survive over a 13-day period (BLI < 5% of initial) apparently due to local xenogeneic responses as well as responses against the vehicle (Matrigel). To determine whether HIP cells could be re-administered with a similar lack of immune activation, the NHPs were re-injected with the same cell type (wild-type or HIP) as the initial injection at day 118 following the initial injections. As before, blood was drawn for analysis prior to re-injection and at days 7 and 13 thereafter (125 and 131 days after first injection, respectively), and cell survival was monitored by BLI. Remarkably, no systemic immune response was observed in the animals re-injected with xenogeneic HIP cells. This indicates that HIP cells can evade immune recognition and activation on multiple doses.

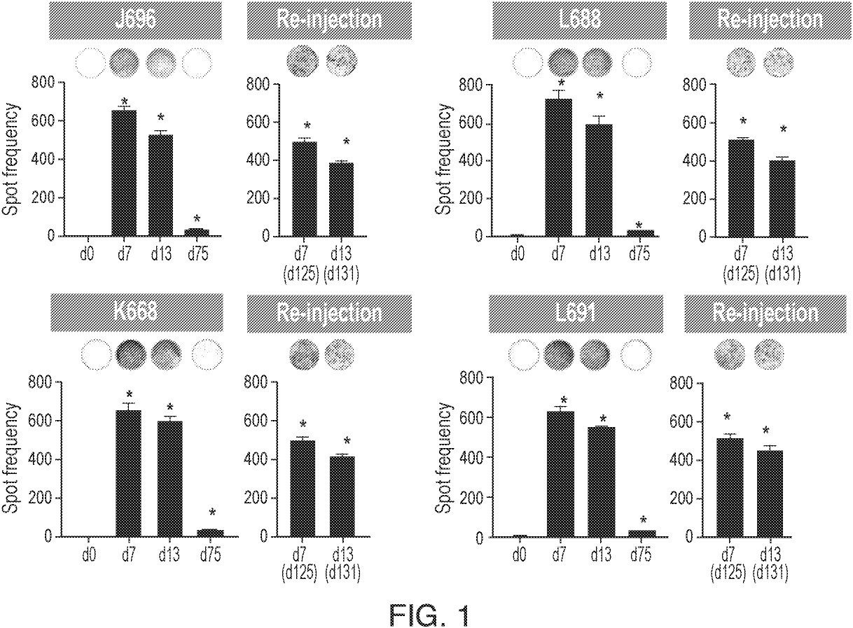

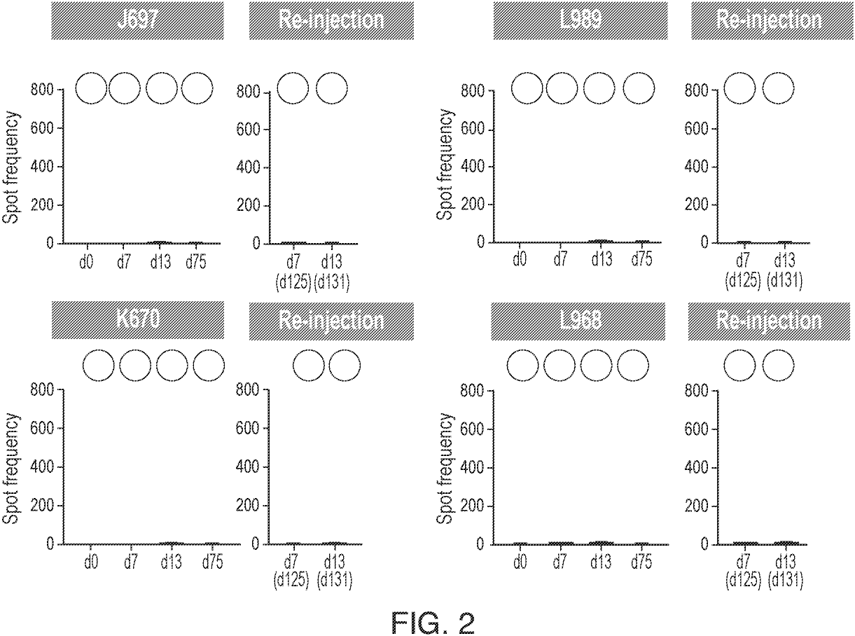

T cell activation. T cell activation in animals administered wild-type and HIP human iPSC was measured by Elispot assays. For uni-directional Elispot assays, recipient PBMCs were isolated from rhesus macaques 0, 7, 13, and 75 days after the first injection and 7 and 13 days after re-injection. T cells were purified from the PBMCs by CD3 MACS-sorting (Miltenyi) and were used as responder cells. Donor cells (wild-type or HIP cells) were mitomycin-treated (50 µg/mL for 30 minutes, Sigma) and used as stimulator cells. 1 x 10⁵ stimulator cells were incubated with 5 x 10⁵ recipient responder T-cells for 36 hours and IFN-y spot frequencies were enumerated using an Elispot plate reader. For the animals administered wild-type cells, Elispot activity observed was highest at day 7 following injection and decreased by day 75. Following re-injection of the wild-type cells, Elispot activity was again highest at day 7 and decrease by day 13 (FIG. 1). These results are indicative of systemic TH1 activation and acute cellular immune response after injection of wild-type cells. By contrast, the animals injected HIP cells had undetectable Elispot activity on both first injection and re-injection, indicating no systemic TH1 activation or cellular immune response to the modified cells (FIG. 2).

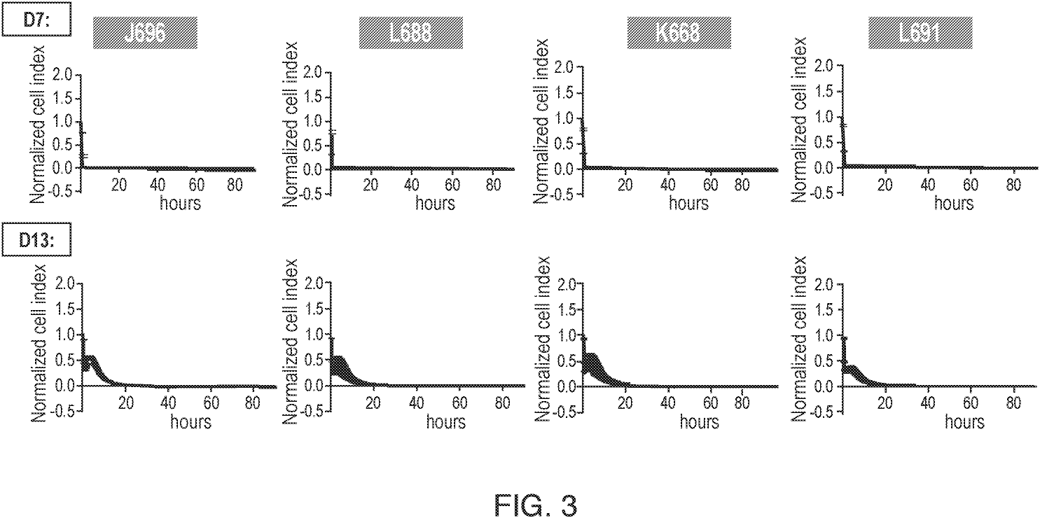

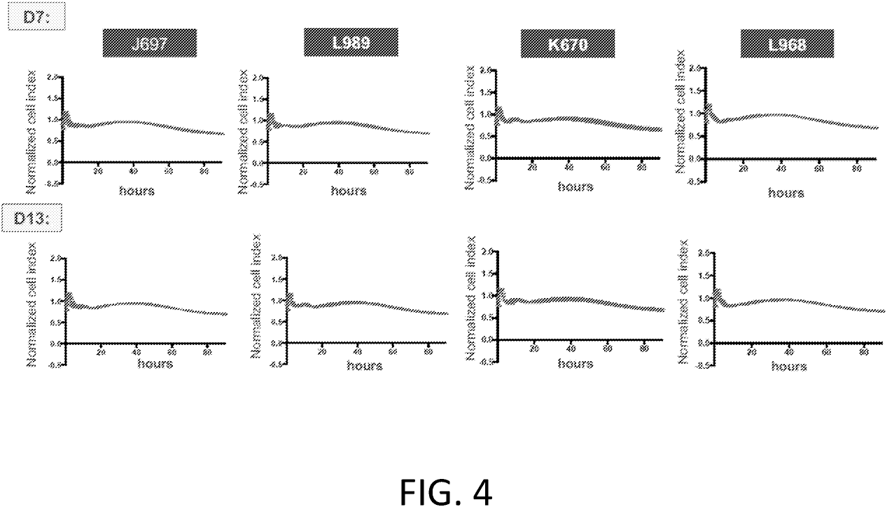

PBMC activation. The ability of immune cells to kill injected cells was also measured. PBMC were isolated from the animals re-injected with wild-type and HIP cells at day 7 and 13 following re-injection, and killing assays were performed on the XCELLIGENCE MP platform (ACEA BioSciences, San Diego, CA.). 96-well E-plates (ACEA BioSciences) were coated with collagen (Sigma-Aldrich) and 4 x 10⁵ wild-type or HIP cells were plated in 100 µl cell specific media. After the Cell Index value reached 0.7, rhesus PBMCs from the subject animals were added with an effector cell to target cell (E:T) ratio of 1:1. As killing control, cells were treated with 2% TRITON X100. As survival control, cells were only incubated with media. Data were standardized and analyzed with the RTCA software (ACEA). PBMC isolated from the animals re-injected with wild-type cells rapidly killed wild-type cells in vitro (FIG. 3). On the other hand, no killing was observed for HIP cells incubated with PBMC isolated from animals re-injected with HIP cells (FIG. 4). Cell killing and survival was confirmed by flow cytometry using LIVE/DEAD staining.

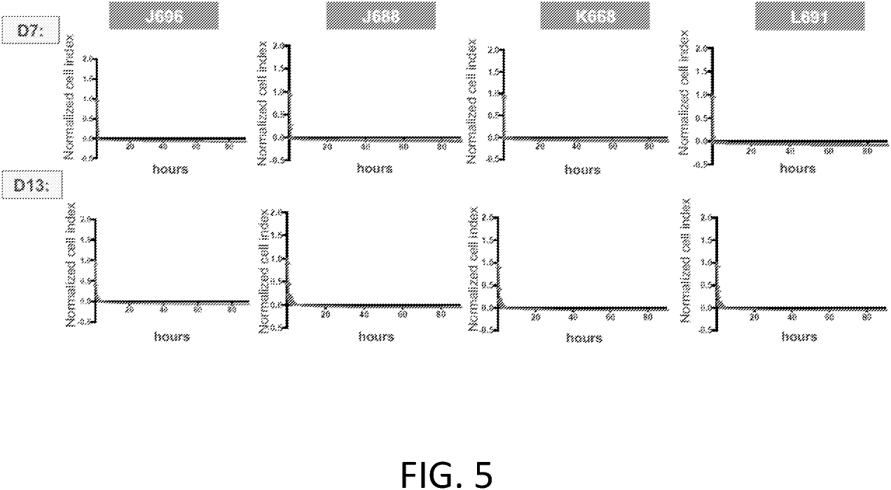

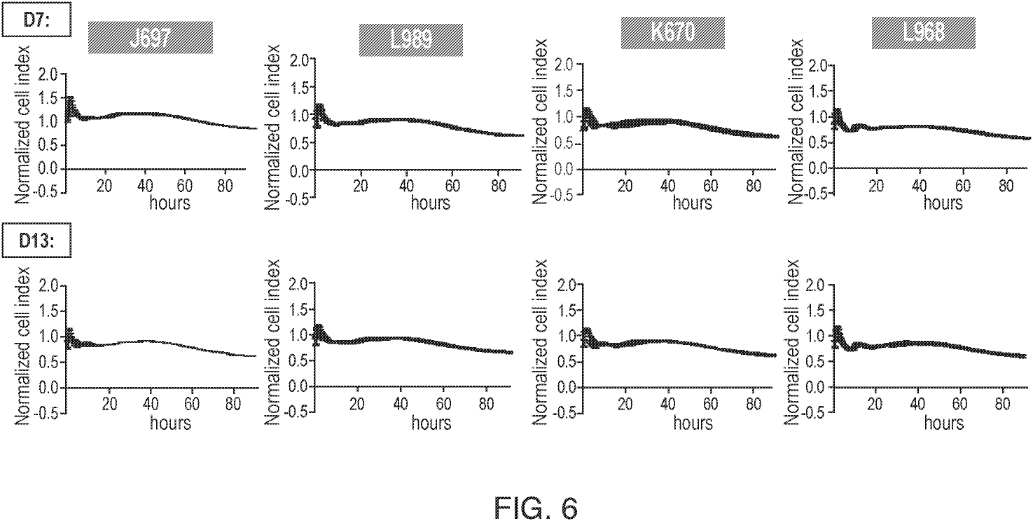

Cytotoxic T cell killing. The ability of cytotoxic T cells to kill donor cells was also assayed. Cytotoxic T cells were isolated from monkey PBMCs by fluorescence activated cell sorting for CD3+CD8+ cells. Similarly to the PBMC assay, cytotoxic T cells isolated from the animals re-injected with wild-type cells rapidly killed wild-type cells in vitro (FIG. 5), whereas no killing was observed when cytotoxic T cells isolated from the animals re-injected with HIP cells were incubated with HIP cells (FIG. 6). Cell killing and survival as above was also confirmed by flow cytometry. These results also support the conclusion that there was no systemic T cell activation in the animals re-injected HIP cells.

NK cell and macrophage killing. Systemic innate immunity by NK cells and macrophages was also assayed in the animals re-injected wild-type or HIP. NK cell killing assays and macrophage killing assays were performed on the XCELLIGENCE MP platform (ACEA BioSciences). 96-well E-plates (ACEA BioSciences) were coated with collagen (Sigma-Aldrich) and 4 x 10⁵ wild-type or HIP cells were plated in 100 µl cell specific media. After the Cell Index value reached 0.7, rhesus NK cells or rhesus macrophages isolated from the treated animals were added with an E:T ratio of 1:1 with or without 1 ng/ml rhesus IL-2 (MyBiosource, San Diego, CA). As a killing control, cells were treated with 2% TRITON X100. No killing was observed by stimulated or unstimulated NK cells or macrophages on wild-type or HIP cells, indicating that CD47 expression on the HIP cells was effective to protect from NK cells and macrophages in the absence of HLA I and HLA II (see Deuse et al., 2019, Nat. Biotechnol., 37:252-258).

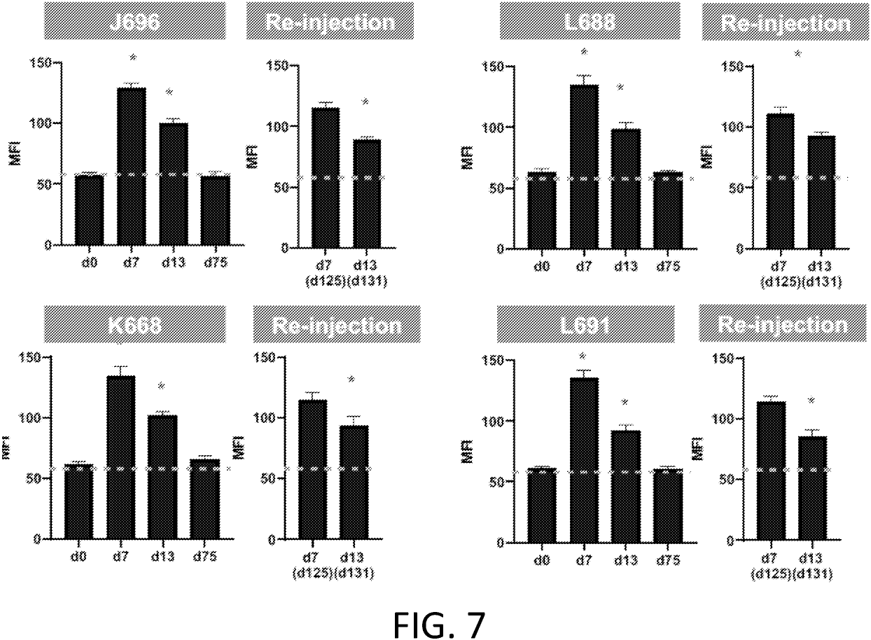

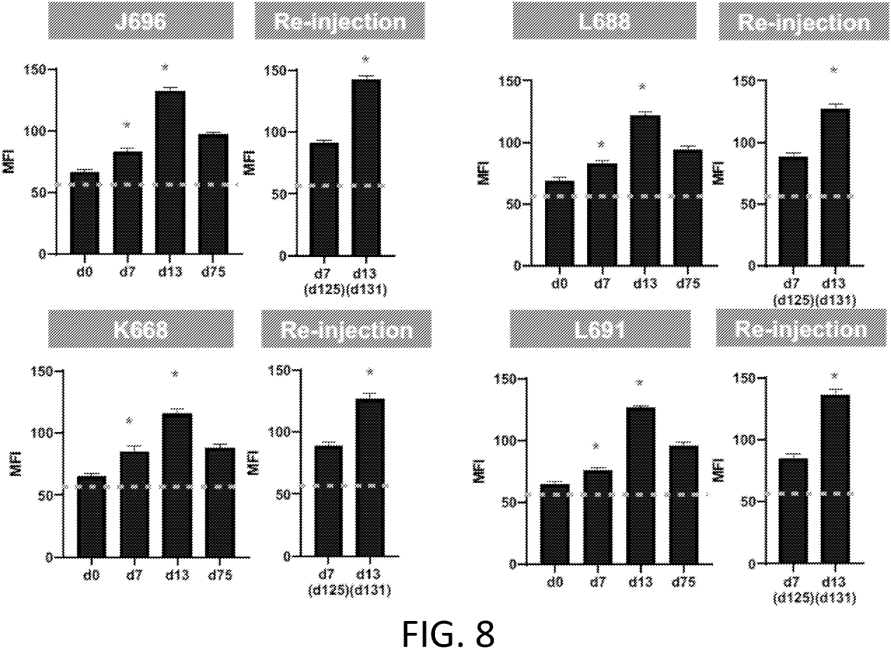

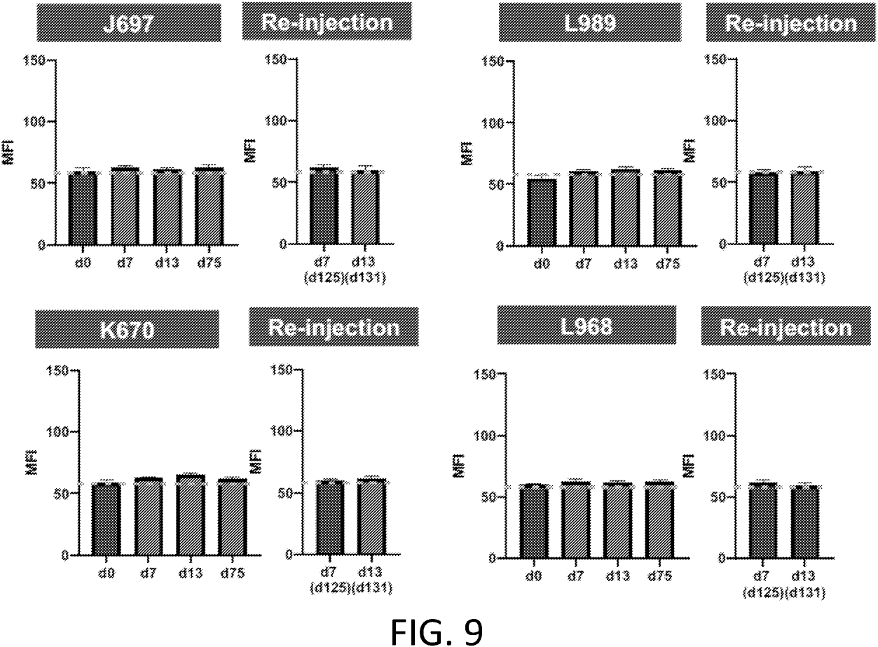

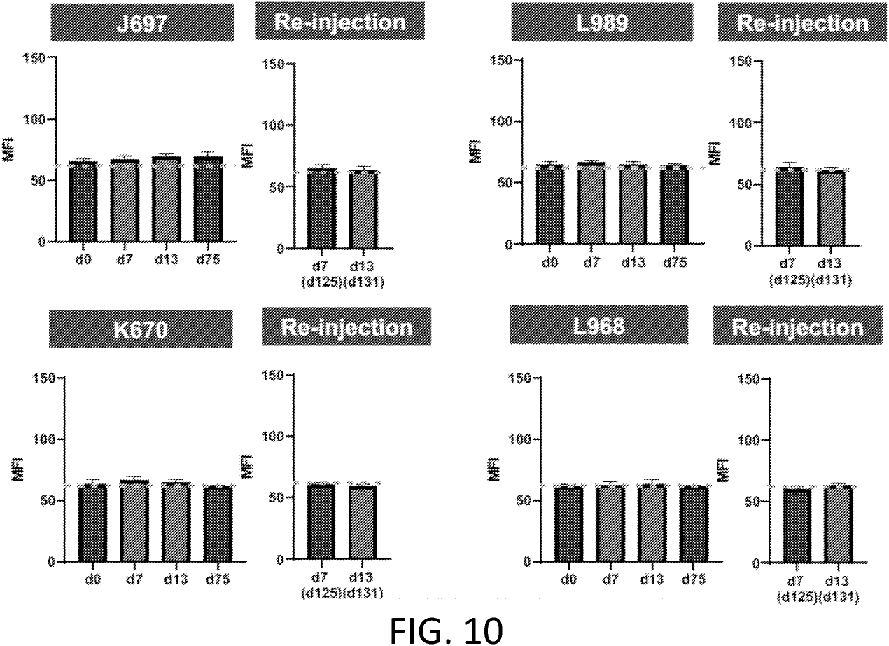

Donor-specific antibody activity. Production of donor-specific antibodies by the animals on first and re-injection with wild-type and HIP cells was also assayed. Sera from recipient monkeys were de-complemented by heating to 56° C. for 30 minutes. Equal amounts of sera and wild-type or HIP cell suspensions (5 x 10⁶ cells/mL) were incubated for 45 minutes at 4° C. Cells were labelled with FITC-conjugated goat anti- IgM or -IgG (BD Bioscience) and analyzed by flow cytometry (BD Bioscience). An increase in donor-specific reactivity was observed at days 7 and 13 following first injection of wild-type cells, and again at days 7 and 13 following re-injection of the wild-type cells, with IgM decreasing from day 7 to 13 and IgG increasing over the same time period, indicative of isotype switching (FIGS. 7-8). By contrast, no increase in donor-specific IgM or IgG reactivity was observed on first injection or re-injection of HIP cells (FIGS. 9-10).

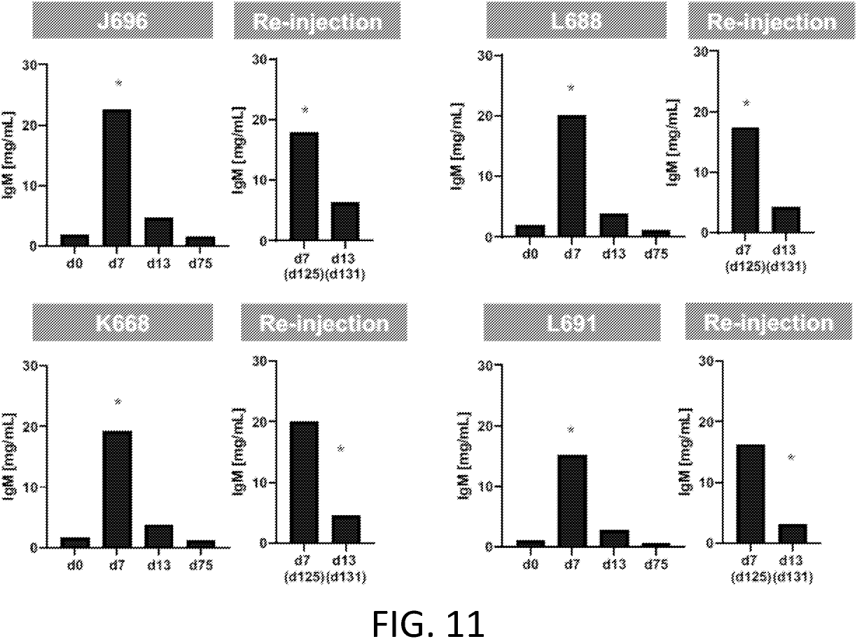

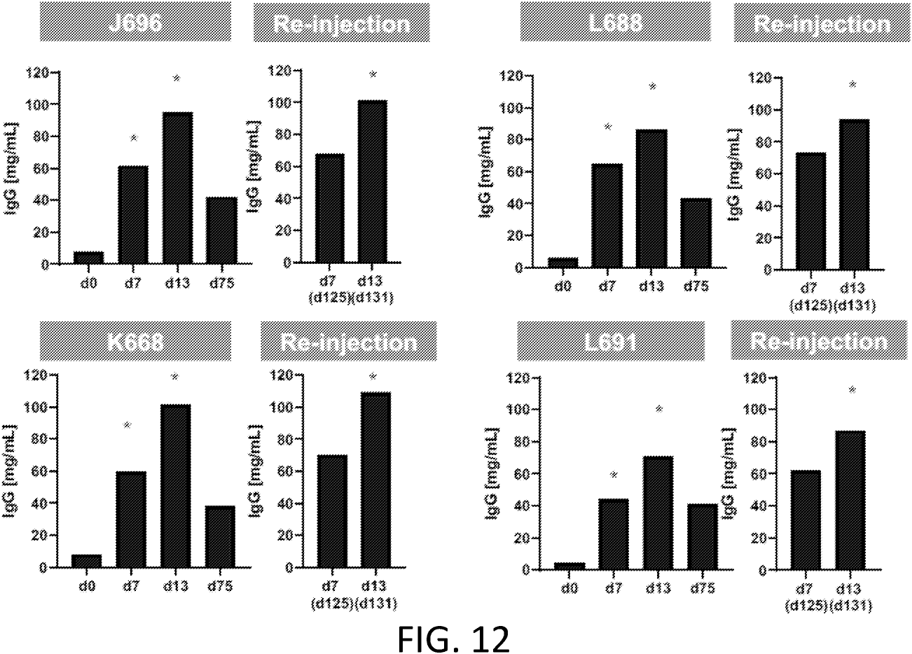

Bulk antibody production. Total antibody production in the animals administered wild-type and HIP cells was also assayed using IgM and IgG ELISA kits (Abcam). After the removal of unbound proteins by washing, anti-IgM antibodies conjugated with horseradish peroxidase (HRP), are added. These enzyme-labeled antibodies form complexes with the previously bound IgM or IgG. The enzyme bound to the immunosorbent is assayed by the addition of a chromogenic substrate, 3,3′,5,5′-tetramethyl-benzidine (TMB). In the animals administered wild-type cells, a sharp increase in total IgM and IgG was observed following both first and re-injection, with the greatest IgM production observed at day 7 and greatest IgG production observed at day 13, again indicating isotype switching (FIGS. 11-12). Strikingly, no increase in total IgM or IgG was observed at any time point in the animals administered HIP cells (FIGS. 13-14). Together, these results indicate a near-total lack of humoral immune response to the HIP cells.

Complement-dependent cytotoxicity (CDC) and antibody-dependent cellular cytotoxicity (ADCC). Further, CDC and ADCC killing assays using serum from the re-injected animals were performed on the XCELLIGENCE MP platform (ACEA BioSciences). 96-well E-plates (ACEA BioSciences) were coated with collagen (Sigma-Aldrich) and 4 x 10⁵ wild-type or HIP cells were plated in 100 µl cell specific media. After the Cell Index value reached 0.7, rhesus NK sera were added; CDC: whole serum. For ADCC: Serum was de-complemented (as described for DSA) and NK cells or macrophages were added with an E:T ratio of 1:1. As a killing control, cells were treated with 2% TRITON X100. Both CDC and ADCC killing were observed with sera of animals administered wild-type cells, whereas no killing (CDC or ADCC) was observed with sera of animals administered HIP cells.

Single-cell sequencing. In addition, single-cell sequencing (10X Genomics) was performed using pooled PBMCs obtained at days 0, 7, and 13 (following both first and re-injection) of animals administered wild-type or HIP cells. Whereas the animals administered wild-type cells showed a marked increase in cytotoxic T cells and NK cells after transplantation, no changes in relative populations of B cells, T cells, NK cells, or cytotoxic T cells though day 13 in the animals administered HIP cells.

Survival of transplanted cells. Although no systemic immune response was observed for animals administered human HIP cells, the cells did not survive due to local xenogeneic responses. Cell viability was monitored throughout the experiment by bioluminescence imaging (BLI). For BLI, D-luciferin firefly potassium salt (375 mg/kg) (Biosynth AG) dissolved in sterile PBS (pH 7.4) (Gibco, Invitrogen) was injected i.v. into anesthetized monkeys. Animals were imaged using the Largo (Spectral Instruments Imaging, Tucson, AZ). Region of interest (ROI) bioluminescence was quantified in units of maximum photons per second per centimeter square per steradian (p/s/cm²/sr). Total luminescence of both wild-type and HIP donor cells decreased throughout the experiment over 13 days following first and re-injection, and dropped below 5% of initial luminescence by day 13. Histopathology analysis performed on cell plugs removed from the animals showed neutrophil infiltration or fibrin (as indicator that neutrophils have been in the area) as well as signs of foreign body reaction and hypersensitivity reaction type IV against the vehicle, indicative of a xenogeneic response against the human cells and allergic reaction to the vehicle, respectively. The allergic and foreign body reaction against the vehicle were confirmed by an additional control monkey injected with only vehicle (no cells), which demonstrated similar histopathological features.

Discussion. Although the HIP cells did not survive, apparently due to xenogeneic responses, the apparent total lack of cellular or humoral adaptive immune response by the animals on first or re-injection is striking and supports the ability of hypoimmune allogeneic cells to be dosed and re-dosed in humans.

All headings and section designations are used for clarity and reference purposes only and are not to be considered limiting in any way. For example, those of skill in the art will appreciate the usefulness of combining various aspects from different headings and sections as appropriate according to the spirit and scope of the present technology described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A method for treating a disorder in a patient comprising administering to the patient a therapeutically effective amount of a population of hypoimmunogenic cells comprising exogenous CD47 polypeptides and reduced expression of MHC class I and/or class II human leukocyte antigens, wherein an initial population of such hypoimmunogenic cells had previously been administered to the patient.

2. The method of claim 1, wherein the hypoimmunogenic cells comprise reduced expression of MHC class I and class II human leukocyte antigens.

3. The method of claim 1 or 2, wherein the hypoimmunogenic cells express the exogenous CD47 polypeptides and reduced expression levels of B2M and/or CIITA.

4. The method of any one of claims 1-3, wherein the hypoimmunogenic cells express the exogenous CD47 polypeptides and reduced expression levels of B2M and CIITA.

5. The method of any one of claims 1-4, wherein the hypoimmunogenic cells are differentiated cells derived from pluripotent stem cells.

6. The method of claim 5, wherein the pluripotent stem cells comprise induced pluripotent stem cells.

7. The method of claim 5 or 6, wherein the differentiated cells are selected from the group consisting of cardiac cells, neural cells, endothelial cells, pancreatic islet cells, retinal pigmented epithelium cells, hepatocytes, thyroid cells, and T cells.

8. The method of any one of claims 1-4, wherein the hypoimmunogenic cells comprise cells derived from primary T cells.

9. The method of claim 8, wherein the cells derived from primary T cells are derived from a pool of T cells comprising primary T cells from one or more, optionally two or more, three or more, four or more, five or more, ten or more, twenty or more, fifty or more, or one hundred or more, subjects different from the patient.

10. The method of claim 8 or 9, wherein the cells derived from primary T cells comprise a chimeric antigen receptor.

11. The method of claim 10, wherein the chimeric antigen receptor (CAR) is selected from the group consisting of:
(a) a first generation CAR comprising an antigen binding domain, a transmembrane domain, and a signaling domain;
(b) a second generation CAR comprising an antigen binding domain, a transmembrane domain, and at least two signaling domains;
(c) a third generation CAR comprising an antigen binding domain, a transmembrane domain, and at least three signaling domains; and
(d) a fourth generation CAR comprising an antigen binding domain, a transmembrane domain, three or four signaling domains, and a domain which upon successful signaling of the CAR induces expression of a cytokine gene.

12. The method of claim 11, wherein the antigen binding domain is selected from the group consisting of
(a) an antigen binding domain targets an antigen characteristic of a neoplastic cell;
(b) an antigen binding domain that targets an antigen characteristic of a T cell;
(c) an antigen binding domain targets an antigen characteristic of an autoimmune or inflammatory disorder;
(d) an antigen binding domain that targets an antigen characteristic of senescent cells;
(e) an antigen binding domain that targets an antigen characteristic of an infectious disease; and
(f) an antigen binding domain that binds to a cell surface antigen of a cell.

13. The method of claim 10 or 11, wherein the antigen binding domain is selected from the group consisting of an antibody, an antigen-binding portion thereof, an scFv, and a Fab.

14. The method of any one of claims 11-13, wherein the antigen binding domain binds to CD19, CD20, CD22, or BCMA.

15. The method of any one of claims 11-14, wherein the transmembrane domain comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

16. The method of any one of claims 11-15, wherein the signaling domain(s) comprises a costimulatory domain(s).

17. The method of claim 16, wherein the costimulatory domains comprise two costimulatory domains that are not the same.

18. The method of claim 16 or 17, wherein the costimulatory domain(s) enhances cytokine production, CAR T cell proliferation, and/or CAR T cell persistence during T cell activation.

19. The method of any one of claims 11-18, wherein the cytokine gene is an endogenous or exogenous cytokine gene to the hypoimmunogenic cells.

20. The method of claim 19, wherein the cytokine gene encodes a pro-inflammatory cytokine.

21. The method of claim 20, wherein the pro-inflammatory cytokine is selected from the group consisting of IL-1, IL-2, IL-9, IL-12, IL-18, TNF, IFN-gamma, and a functional fragment thereof.

22. The method of any one of claims 11-21, wherein the domain which upon successful signaling of the CAR induces expression of the cytokine gene comprises a transcription factor or functional domain or fragment thereof.

23. The method of any one of claims 10-22, wherein the CAR comprises a CD3 zeta domain or an immunoreceptor tyrosine-based activation motif (ITAM), or functional variant thereof.

24. The method of any one of claims 10-23, wherein the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

25. The method of any one of claims 10-24, wherein the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

26. The method of any one of claims 10-25, wherein the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

27. The method of any one of claims 10-24, wherein the CAR comprises a (i) an anti-CD19 scFv; (ii) a CD8α hinge and transmembrane domain or functional variant thereof; (iii) a 4-1BB costimulatory domain or functional variant thereof; and (iv) a CD3ζ signaling domain or functional variant thereof.

28. The method of any one of claims 8-27, wherein the cells derived from primary T cells comprise reduced expression of an endogenous T cell receptor.

29. The method of any one of claims 8-28, wherein the cells derived from primary T cells comprise reduced expression of cytotoxic T-lymphocyte-associated protein 4 (CTLA4) and/or programmed cell death (PD1).

30. The method of any one of claims 1-29, wherein the population of the hypoimmunogenic cells is administered at least 3 days or more after the initial administration, optionally at least 4 days, 5 days, 6 days, 7 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 1 month, 2 months, 3 months, 4 months, 5 months, 6 months, 12 months, 18 months, 24 months, 30 months, 36 months, 48 months, 54 months, or 60 months or more.

31. The method of any one of claims 1-30, wherein the population of the hypoimmunogenic cells is administered at least 3 days to at least 7 days or more after the initial administration.

32. The method of any one of claims 1-30, wherein the population of the hypoimmunogenic cells is administered least 1 month or more after the initial administration.

33. The method of any one of claims 1-30, wherein the population of the hypoimmunogenic cells is administered at least 2 months or more after the initial administration.

34. The method of any one of claims 1-33, wherein upon the initial and/or subsequent administrations, the population of hypoimmunogenic cells elicits a reduced level of immune activation or no immune activation in the patient.

35. The method of any one of claims 1-34, wherein upon the initial and/or subsequent administrations, the population of hypoimmunogenic cells elicits a reduced level of systemic TH1 activation or no systemic TH1 activation in the patient.

36. The method of any one of claims 1-35, wherein upon the initial and/or subsequent administrations, the population of hypoimmunogenic cells elicits a reduced level of immune activation of peripheral blood mononuclear cells (PBMCs) or no immune activation of PBMCs in the patient.

37. The method of any one of claims 1-36, wherein upon the initial and/or subsequent administrations, the population of hypoimmunogenic cells elicits a reduced level of donor-specific IgG antibodies or no donor specific IgG antibodies against the hypoimmunogenic cells in the patient.

38. The method of any one of claims 1-37, wherein upon the initial and/or subsequent administrations, the population of hypoimmunogenic cells elicits a reduced level of IgM and IgG antibody production or no IgM and IgG antibody production against the hypoimmunogenic cells in the patient.

39. The method of any one of claims 1-38, wherein upon administration, the population of hypoimmunogenic cells elicits a reduced level of cytotoxic T cell killing or no cytotoxic T cell killing of the hypoimmunogenic cells of the hypoimmunogenic cells in the patient.

40. The method of any one of claims 1-39, wherein the population of hypoimmunogenic cells of the initial administration are no longer present in the patient at the subsequent administration.

41. The method of any one of claims 1-40, wherein the patient is not administered an immunosuppressive agent at least 3 days or more before or after the initial administration of the population of hypoimmunogenic cells.

42. The method of any one of claims 1-41, wherein the patient is not administered an immunosuppressive agent at least 3 days or more before or after the initial subsequent administration of the population of hypoimmunogenic cells.

43. The method of any one of claims 1-42, wherein the hypoimmunogenic cell is selected from the group consisting a pluripotent stem cell, an induced pluripotent stem cell, a T cell differentiated from an induced pluripotent stem cell, a primary T cell, and a cell derived from a primary T cell, and the hypoimmunogenic cell is a B2M^{indel/indel}, CIITA^{indel/indel} cell comprising the exogenous CD47 polypeptides and optionally a CAR.

44. A method for treating a disorder in a patient comprising administering to the patient therapeutically effective amounts of a population of hypoimmunogenic cells in a dosing regimen comprising a first administration, a recovery period and a second administration, wherein the hypoimmunogenic cells comprise exogenous CD47 polypeptides and reduced expression of MHC class I and/or class II human leukocyte antigens.

45. The method of claim 44, wherein the hypoimmunogenic cells further comprise reduced expression of MHC class I and II human leukocyte antigens.

46. The method of claim 44 or 45, wherein the hypoimmunogenic cells express the exogenous CD47 polypeptide and reduced expression levels of B2M and/or CIITA.

47. The method of any one of claims 44-46, wherein the hypoimmunogenic cells express the exogenous CD47 polypeptide and reduced expression levels of B2M and CIITA.

48. The method of any one of claims 44-47, wherein the hypoimmunogenic cells are differentiated cells derived from pluripotent stem cells.

49. The method of claim 48, wherein the pluripotent stem cells comprise induced pluripotent stem cells.

50. The method of claim 48 or 49, wherein the differentiated cells are selected from the group consisting of cardiac cells, neural cells, endothelial cells, pancreatic islet cells, retinal pigmented epithelium cells, hepatocytes, thyroid cells, and T cells.

51. The method of any one of claims 44-47, wherein the hypoimmunogenic cells comprise cells derived from primary T cells.

52. The method of claim 51, wherein the cells derived from primary T cells are derived from a pool of T cells comprising primary T cells from one or more subjects different from the patient.

53. The method of claim 51 or 52, wherein the cells derived from primary T cells comprise a chimeric antigen receptor.

54. The method of claim 53, wherein the chimeric antigen receptor (CAR) is selected from the group consisting of:
(a) a first generation CAR comprising an antigen binding domain, a transmembrane domain, and a signaling domain;
(b) a second generation CAR comprising an antigen binding domain, a transmembrane domain, and at least two signaling domains;
(c) a third generation CAR comprising an antigen binding domain, a transmembrane domain, and at least three signaling domains; and
(d) a fourth generation CAR comprising an antigen binding domain, a transmembrane domain, three or four signaling domains, and a domain which upon successful signaling of the CAR induces expression of a cytokine gene.

55. The method of claim 54, wherein the antigen binding domain is selected from the group consisting of
(a) an antigen binding domain targets an antigen characteristic of a neoplastic cell;
(b) an antigen binding domain that targets an antigen characteristic of a T cell,
(c) an antigen binding domain targets an antigen characteristic of an autoimmune or inflammatory disorder;
(d) an antigen binding domain that targets an antigen characteristic of senescent cells;
(e) an antigen binding domain that targets an antigen characteristic of an infectious disease; and
(f) an antigen binding domain that binds to a cell surface antigen of a cell.

56. The method of claim 54 or 55, wherein the antigen binding domain is selected from the group consisting of an antibody, an antigen-binding portion thereof, an scFv, and a Fab.

57. The method of any one of claims 54-56, wherein the antigen binding domain binds to CD19, CD20, CD22, or BCMA.

58. The method of any one of claims 54-57, wherein the transmembrane domain comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

59. The method of any one of claims 54-58, wherein the signaling domain(s) comprises a costimulatory domain(s).

60. The method of claim 59, wherein the costimulatory domains comprise two costimulatory domains that are not the same.

61. The method of claim 59 or 60, wherein the costimulatory domain(s) enhances cytokine production, CAR T cell proliferation, and/or CAR T cell persistence during T cell activation.

62. The method of any one of claims 54-61, wherein the cytokine gene is an endogenous or exogenous cytokine gene to the hypoimmunogenic cells.

63. The method of claim 62, wherein the cytokine gene encodes a pro-inflammatory cytokine.

64. The method of claim 63, wherein the pro-inflammatory cytokine is selected from the group consisting of IL-1, IL-2, IL-9, IL-12, IL-18, TNF, IFN-gamma, and a functional fragment thereof.

65. The method of any one of claims 54-64, wherein the domain which upon successful signaling of the CAR induces expression of the cytokine gene comprises a transcription factor or functional domain or fragment thereof.

66. The method of any one of claims 53-65, wherein the CAR comprises a CD3 zeta domain or an immunoreceptor tyrosine-based activation motif (ITAM), or functional variant thereof.

67. The method of any one of claims 53-66, wherein the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

68. The method of any one of claims 53-67, wherein the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

69. The method of any one of claims 53-68, wherein the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

70. The method of any one of claims 53-67, wherein the CAR comprises a (i) an anti-CD19 scFv; (ii) a CD8α hinge and transmembrane domain or functional variant thereof; (iii) a 4-1BB costimulatory domain or functional variant thereof; and (iv) a CD3ζ signaling domain or functional variant thereof.

71. The method of any one of claim 51-70, wherein the cells derived from primary T cells comprise reduced expression of an endogenous T cell receptor.

72. The method of any one of claim 51-71, wherein the cells derived from primary T cells comprise reduced expression of cytotoxic T-lymphocyte-associated protein 4 (CTLA4) and/or programmed cell death (PD1).

73. The method of any one of claims 44-72, wherein the recovery period comprises at least 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 1 month, 2 months, 3 months, 4 months, 5 months, 6 months, 12 months, 18 months, 24 months, 30 months, 36 months, 48 months, 54 months, or 60 months or more.

74. The method of any one of claims 44-72, wherein the recovery period comprises at least 3 days to at least 7 days or more.

75. The method of any one of claims 44-72, wherein the recovery period comprises at least 1 month or more.

76. The method of any one of claims 44-73, wherein the recovery period comprises at least 2 months or more.

77. The method of any one of claims 44-76, wherein the second administration is initiated when the population of hypoimmunogenic cells from the first administration is no longer detectable in the patient.

78. The method of any one of claims 44-77, wherein upon the first and/or second administrations, the population of hypoimmunogenic cells elicits a reduced level of immune activation or no immune activation in the patient.

79. The method of any one of claims 44-78, wherein upon the first and/or second administrations, the population of hypoimmunogenic cells elicits a reduced level of systemic TH1 activation or no systemic TH1 activation in the patient.

80. The method of any one of claims 44-79, wherein upon the first and/or second administrations, the population of hypoimmunogenic cells elicits a reduced level of immune activation of peripheral blood mononuclear cells (PBMCs) or no immune activation of PBMCs in the patient.

81. The method of any one of claims 44-80, wherein upon the first and/or second administrations, the population of hypoimmunogenic cells elicits a reduced level of donor-specific IgG antibodies or no donor-specific IgG antibodies against the hypoimmunogenic cells in the patient.

82. The method of any one of claims 44-81, wherein upon the first and/or second administrations, the population of hypoimmunogenic cells elicits a reduced level of IgM and IgG antibody production or no IgM and IgG antibody production against the hypoimmunogenic cells in the patient.

83. The method of any one of claims 44-82, wherein upon the first and/or second administrations, the population of hypoimmunogenic cells elicits a reduced level of cytotoxic T cell killing or no cytotoxic T cell killing of the hypoimmunogenic cells in the patient.

84. The method of any one of claims 44-83, wherein the patient is not administered an immunosuppressive agent at least 3 days or more before or after the first administration of the population of hypoimmunogenic cells.

85. The method of any one of claims 44-84, wherein the patient is not administered an immunosuppressive agent at least 3 days or more before or after the second administration of the population of hypoimmunogenic cells.

86. The method of any one of claims 44-85, wherein the patient is not administered an immunosuppressive agent during the recovery period.

87. The method of any one of claims 44-86, wherein the hypoimmunogenic cell is selected from the group consisting a pluripotent stem cell, an induced pluripotent stem cell, a T cell differentiated from an induced pluripotent stem cell, a primary T cell, and a cell derived from a primary T cell, and the hypoimmunogenic cell is a B2M^{indel/indel}, CIITA^{indel/indel} cell comprising the exogenous CD47 polypeptides and optionally a CAR.

88. A method for treating a disorder in a patient by administering cells that do not trigger a systemic acute cellular immune response in the patient, the method comprising:
a) administering a therapeutically effective amount of a first population of cells to the patient; and
b) administering a therapeutically effective amount of a second population of cells to the patient following a recovery period after step (a),
wherein the cells of the first and second populations of cells comprise exogenous CD47 polypeptides and reduced expression of MHC class I and/or II human leukocyte antigens, and wherein the recovery period comprises at least 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 1 month, 2 months, 3 months, 4 months, 5 months, 6 months, 12 months, 18 months, 24 months, 30 months, 36 months, 48 months, 54 months, or 60 months or more.

89. The method of claim 88, wherein the cells of the first and second populations comprise reduced expression of MHC class I and MHC class II human leukocyte antigens.

90. The method of claim 88 or 89, wherein the cells of the first and second populations comprise the exogenous CD47 polypeptides and reduced expression levels of B2M and/or CIITA.

91. The method of any one of claims 88-90, wherein the cells of the first and second populations comprise the exogenous CD47 polypeptides and reduced expression levels of B2M and CIITA.

92. The method of any one of claims 88-91, wherein the first and second populations of cells comprise differentiated cells derived from pluripotent stem cells.

93. The method of claim 92, wherein the pluripotent stem cells comprise induced pluripotent stem cells.

94. The method of claim 92 or 93, wherein the differentiated cells are selected from the group consisting of cardiac cells, neural cells, endothelial cells, pancreatic islet cells, retinal pigmented epithelium cells, hepatocytes, thyroid cells and T cells.

95. The method of any one of claims 88-91, wherein the first and second populations of cells comprises cells derived from primary T cells.

96. The method of claim 95, wherein the cells derived from primary T cells are derived from a pool of T cells comprising primary T cells from one or more, optionally, two or more, three or more, four or more, five or more, ten or more, twenty or more, fifty or more, or one hundred or more, subjects different from the patient.

97. The method of claim 95 or 96, wherein the cells derived from primary T cells comprise a chimeric antigen receptor.

98. The method of claim 97, wherein the chimeric antigen receptor (CAR) is selected from the group consisting of
(a) a first generation CAR comprising an antigen binding domain, a transmembrane domain, and a signaling domain;
(b) a second generation CAR comprising an antigen binding domain, a transmembrane domain, and at least two signaling domains;
(c) a third generation CAR comprising an antigen binding domain, a transmembrane domain, and at least three signaling domains; and
(d) a fourth generation CAR comprising an antigen binding domain, a transmembrane domain, three or four signaling domains, and a domain which upon successful signaling of the CAR induces expression of a cytokine gene.

99. The method of claim 98, wherein the antigen binding domain is selected from the group consisting of:
(a) an antigen binding domain targets an antigen characteristic of a neoplastic cell;
(b) an antigen binding domain that targets an antigen characteristic of a T cell,
(c) an antigen binding domain targets an antigen characteristic of an autoimmune or inflammatory disorder;
(d) an antigen binding domain that targets an antigen characteristic of senescent cells;
(e) an antigen binding domain that targets an antigen characteristic of an infectious disease; and
(f) an antigen binding domain that binds to a cell surface antigen of a cell.

100. The method of claim 98 or 99, wherein the antigen binding domain is selected from the group consisting of an antibody, an antigen-binding portion thereof, an scFv, and a Fab.

101. The method of any one of claims 98-100, wherein the antigen binding domain binds to CD19, CD20, CD22, or BCMA.

102. The method of any one of claims 98-101, wherein the transmembrane domain comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

103. The method of any one of claims 98-102, wherein the signaling domain(s) comprises a costimulatory domain(s).

104. The method of claim 103, wherein the costimulatory domains comprise two costimulatory domains that are not the same.

105. The method of claim 103 or 104, wherein the costimulatory domain(s) enhances cytokine production, CAR T cell proliferation, and/or CAR T cell persistence during T cell activation.

106. The method of any one of claims 97-105, wherein the cytokine gene is an endogenous or exogenous cytokine gene to the hypoimmunogenic cells.

107. The method of claim 106, wherein the cytokine gene encodes a pro-inflammatory cytokine.

108. The method of claim 107, wherein the pro-inflammatory cytokine is selected from the group consisting of IL-1, IL-2, IL-9, IL-12, IL-18, TNF, IFN-gamma, and a functional fragment thereof.

109. The method of any one of claims 97-108, wherein the domain which upon successful signaling of the CAR induces expression of the cytokine gene comprises a transcription factor or functional domain or fragment thereof.

110. The method of any one of claims 96-109, wherein the CAR comprises a CD3 zeta domain or an immunoreceptor tyrosine-based activation motif (ITAM), or functional variant thereof.

111. The method of any one of claims 96-110, wherein the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

112. The method of any one of claims 96-111, wherein the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

113. The method of any one of claims 96-112, wherein the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

114. The method of any one of claims 96-111, wherein the CAR comprises a (i) an anti-CD19 scFv; (ii) a CD8α hinge and transmembrane domain or functional variant thereof; (iii) a 4-1BB costimulatory domain or functional variant thereof; and (iv) a CD3ζ signaling domain or functional variant thereof.

115. The method of any one of claims 95-114, wherein the cells derived from primary T cells comprise reduced expression of an endogenous T cell receptor.

116. The method of any one of claims 95-115, wherein the cells derived from primary T cells comprise reduced expression of cytotoxic T-lymphocyte-associated protein 4 (CTLA4) and/or programmed cell death (PD1).

117. The method of any one of claims 88-116, wherein the recovery period comprises at least 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 1 month, 2 months, 3 months, 4 months, 5 months, 6 months, 12 months, 18 months, 24 months, 30 months, 36 months, 48 months, 54 months, or 60 months or more.

118. The method of any one of claims 88-117, wherein the step (b) is performed when the first population of cells is no longer detectable in the patient.

119. The method of any one of claims 88-118, wherein the first and/or second population of cells elicits a reduced level of immune activation or no immune activation in the patient.

120. The method of any one of claims 88-119, wherein the first and/or second population of cells elicits a reduced level of systemic TH1 activation or no systemic TH1 activation in the patient.

121. The method of any one of claims 88-120, wherein the first and/or second population of cells elicits a reduced level of immune activation of peripheral blood mononuclear cells (PBMCs) or no immune activation of PBMCs in the patient.

122. The method of any one of claims 88-121, wherein the first and/or second population of cells elicits a reduced level of donor-specific IgG antibodies or no donor-specific IgG antibodies against the administered cells in the patient.

123. The method of any one of claims 88-122, wherein the first and/or second population of cells elicits a reduced level of IgM and IgG antibody production or no IgM and IgG antibody production against the administered cells in the patient.

124. The method of any one of claims 88-123, wherein the first and/or second population of cells elicits a reduced level of cytotoxic T cell killing or no cytotoxic T cell killing of the administered cells in the patient.

125. The method of any one of claims 88-124, wherein the patient is not administered an immunosuppressive agent at least 3 days or more before or after the administration of the first population of cells.

126. The method of any one of claims 88-125, wherein the patient is not administered an immunosuppressive agent at least 3 days or more before or after the administration of the second population of cells.

127. The method of any one of claims 88-126, wherein the patient is not administered an immunosuppressive agent during the recovery period.

128. The method of any one of claims 88-127, wherein the hypoimmunogenic cell is selected from the group consisting a pluripotent stem cell, an induced pluripotent stem cell, a T cell differentiated from an induced pluripotent stem cell, a primary T cell, and a cell derived from a primary T cell, and the hypoimmunogenic cell is a B2M^{indel/indel}, CIITA^{indel/indel} cell comprising the exogenous CD47 polypeptides and optionally a CAR.

129. Use of a population of hypoimmunogenic cells comprising exogenous CD47 polypeptides and reduced expression of MHC class I and/or class II human leukocyte antigens for treatment of a disorder in a patient, wherein the patient has previously received an initial population of such hypoimmunogenic cells.

130. Use of a population of hypoimmunogenic cells comprising exogenous CD47 polypeptides and reduced expression of MHC class I and class II human leukocyte antigens for treatment of a disorder in a patient, wherein the patient has previously received an initial population of such hypoimmunogenic cells.

131. Use of a population of hypoimmunogenic cells comprising exogenous CD47 polypeptides and reduced levels of B2M and CIITA polypeptides for treatment of a disorder in a patient, wherein the patient has previously received an initial population of such hypoimmunogenic cells.

132. Use of a population of hypoimmunogenic cells comprising exogenous CD47 polypeptides, a genomic modification of the B2M gene, and a genomic modification of the CIITA gene for treatment of a disorder in a patient, wherein the patient has previously received an initial population of such hypoimmunogenic cells.

133. The use of the population of hypoimmunogenic cells of any one of claims 129-132, wherein the population of hypoimmunogenic cells comprises differentiated cells derived from pluripotent stem cells.

134. The use of the population of hypoimmunogenic cells of claim 133, wherein the pluripotent stem cells comprise induced pluripotent stem cells.

135. The use of the population of hypoimmunogenic cells of claim 133 or 134, wherein the differentiated cells are selected from the group consisting of cardiac cells, neural cells, endothelial cells, pancreatic islet cells, retinal pigmented epithelium cells, hepatocytes, thyroid cells, and T cells.

136. The use of the population of hypoimmunogenic cells of any one of claims 129-132, wherein the population of hypoimmunogenic cells comprises cells derived from primary T cells.

137. The use of the population of hypoimmunogenic cells of claim 136, wherein the cells derived from primary T cells are derived from a pool of T cells comprising primary T cells from one or more subjects different from the patient.

138. The use of the population of hypoimmunogenic cells of claim 136 or 137, wherein the cells derived from primary T cells comprise a chimeric antigen receptor (CAR).

139. The use of the population of hypoimmunogenic cells of claim 138, wherein the chimeric antigen receptor (CAR) is selected from the group consisting of:
(a) a first generation CAR comprising an antigen binding domain, a transmembrane domain, and a signaling domain;
(b) a second generation CAR comprising an antigen binding domain, a transmembrane domain, and at least two signaling domains;
(c) a third generation CAR comprising an antigen binding domain, a transmembrane domain, and at least three signaling domains; and
(d) a fourth generation CAR comprising an antigen binding domain, a transmembrane domain, three or four signaling domains, and a domain which upon successful signaling of the CAR induces expression of a cytokine gene.

140. The use of the population of hypoimmunogenic cells of claim 139, wherein the antigen binding domain is selected from the group consisting of:
(a) an antigen binding domain targets an antigen characteristic of a neoplastic cell;
(b) an antigen binding domain that targets an antigen characteristic of a T cell,
(c) an antigen binding domain targets an antigen characteristic of an autoimmune or inflammatory disorder;
(d) an antigen binding domain that targets an antigen characteristic of senescent cells;
(e) an antigen binding domain that targets an antigen characteristic of an infectious disease; and
(f) an antigen binding domain that binds to a cell surface antigen of a cell.

141. The use of the population of hypoimmunogenic cells of claim 139 or 140, wherein the antigen binding domain is selected from the group consisting of an antibody, an antigen-binding portion thereof, an scFv, and a Fab.

142. The use of the population of hypoimmunogenic cells of any one of claims 139-141, wherein the antigen binding domain binds to CD19, CD20, CD22, or BCMA.

143. The use of the population of hypoimmunogenic cells of any one of claims 139-142, wherein the transmembrane domain comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

144. The use of the population of hypoimmunogenic cells of any one of claims 139-143, wherein the signaling domain(s) comprises a costimulatory domain(s).

145. The use of the population of hypoimmunogenic cells of claim 144, wherein the costimulatory domains comprise two costimulatory domains that are not the same.

146. The use of the population of hypoimmunogenic cells of claim 144 or 145, wherein the costimulatory domain(s) enhances cytokine production, CAR T cell proliferation, and/or CAR T cell persistence during T cell activation.

147. The use of the population of hypoimmunogenic cells of any one of claims 139-146, wherein the cytokine gene is an endogenous or exogenous cytokine gene to the hypoimmunogenic cells.

148. The use of the population of hypoimmunogenic cells of claim 147, wherein the cytokine gene encodes a pro-inflammatory cytokine.

149. The use of the population of hypoimmunogenic cells of claim 148, wherein the pro-inflammatory cytokine is selected from the group consisting of IL-1, IL-2, IL-9, IL-12, IL-18, TNF, IFN-gamma, and a functional fragment thereof.

150. The use of the population of hypoimmunogenic cells of any one of claims 139-149, wherein the domain which upon successful signaling of the CAR induces expression of the cytokine gene comprises a transcription factor or functional domain or fragment thereof.

151. The use of the population of hypoimmunogenic cells of any one of claims 138-150, wherein the CAR comprises a CD3 zeta domain or an immunoreceptor tyrosine-based activation motif (ITAM), or functional variant thereof.

152. The use of the population of hypoimmunogenic cells of any one of claims 138-151, wherein the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

153. The use of the population of hypoimmunogenic cells of any one of claims 138-152, wherein the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

154. The use of the population of hypoimmunogenic cells of any one of claims 138-153, wherein the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

155. The use of the population of hypoimmunogenic cells of any one of claims 96-152, wherein the CAR comprises a (i) an anti-CD19 scFv; (ii) a CD8α hinge and transmembrane domain or functional variant thereof; (iii) a 4-1BB costimulatory domain or functional variant thereof; and (iv) a CD3ζ signaling domain or functional variant thereof.

156. The use of the population of hypoimmunogenic cells of any one of claims 136-138, wherein the cells derived from primary T cells comprise reduced expression of an endogenous T cell receptor.

157. The use of the population of hypoimmunogenic cells of any one of claims 136-156, wherein the cells derived from primary T cells comprise reduced expression of cytotoxic T-lymphocyte-associated protein 4 (CTLA4) and/or programmed cell death (PD1).

158. The use of the population of hypoimmunogenic cells of any one of claims 138-157, wherein the chimeric antigen receptor (CAR) is selected from the group consisting of:
(a) a first generation CAR comprising an antigen binding domain, a transmembrane domain, and a signaling domain;
(b) a second generation CAR comprising an antigen binding domain, a transmembrane domain, and at least two signaling domains;
(c) a third generation CAR comprising an antigen binding domain, a transmembrane domain, and at least three signaling domains; and
(d) a fourth generation CAR comprising an antigen binding domain, a transmembrane domain, three or four signaling domains, and a domain which upon successful signaling of the CAR induces expression of a cytokine gene.

159. The use of the population of hypoimmunogenic cells of claim 158, wherein the antigen binding domain is selected from the group consisting of:
(a) an antigen binding domain targets an antigen characteristic of a neoplastic cell;
(b) an antigen binding domain that targets an antigen characteristic of a T cell,
(c) an antigen binding domain targets an antigen characteristic of an autoimmune or inflammatory disorder;
(d) an antigen binding domain that targets an antigen characteristic of senescent cells;
(e) an antigen binding domain that targets an antigen characteristic of an infectious disease; and
(f) an antigen binding domain that binds to a cell surface antigen of a cell.

160. The use of the population of hypoimmunogenic cells of claim 159, wherein the antigen binding domain is selected from the group consisting of an antibody, an antigen-binding portion thereof, an scFv, and a Fab.

161. The use of the population of hypoimmunogenic cells of claim 159 or 160, wherein the antigen binding domain binds to CD19, CD20, CD22, or BCMA.

162. The use of the population of hypoimmunogenic cells of any one of claims 158-161, wherein the transmembrane domain comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

163. The use of the population of hypoimmunogenic cells of any one of claims 158-162, wherein the signaling domain(s) comprises a costimulatory domain(s).

164. The use of the population of hypoimmunogenic cells of claim 163, wherein the costimulatory domains comprise two costimulatory domains that are not the same.

165. The use of the population of hypoimmunogenic cells of claim 163 or 164, wherein the costimulatory domain(s) enhances cytokine production, CAR T cell proliferation, and/or CAR T cell persistence during T cell activation.

166. The use of the population of hypoimmunogenic cells of any one of claims 158-165, wherein the cytokine gene is an endogenous or exogenous cytokine gene to the hypoimmunogenic cells.

167. The use of the population of hypoimmunogenic cells of claim 166, wherein the cytokine gene encodes a pro-inflammatory cytokine.

168. The use of the population of hypoimmunogenic cells of claim 167, wherein the pro-inflammatory cytokine is selected from the group consisting of IL-1, IL-2, IL-9, IL-12, IL-18, TNF, IFN-gamma, and a functional fragment thereof.

169. The use of the population of hypoimmunogenic cells of any one of claims 158-168, wherein the domain which upon successful signaling of the CAR induces expression of the cytokine gene comprises a transcription factor or functional domain or fragment thereof.

170. The use of the population of hypoimmunogenic cells of any one of claims 138-169, wherein the CAR comprises a CD3 zeta domain or an immunoreceptor tyrosine-based activation motif (ITAM), or functional variant thereof.

171. The use of the population of hypoimmunogenic cells of any one of claims 138-170, wherein the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

172. The use of the population of hypoimmunogenic cells of any one of claims 138-171, wherein the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

173. The use of the population of hypoimmunogenic cells of any one of claims 138-172, wherein the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

174. The use of the population of hypoimmunogenic cells of any one of claims 138-171, wherein the CAR comprises a (i) an anti-CD19 scFv; (ii) a CD8α hinge and transmembrane domain or functional variant thereof; (iii) a 4-1BB costimulatory domain or functional variant thereof; and (iv) a CD3ζ signaling domain.

175. The use of any one of claims 138-171, wherein the hypoimmunogenic cell is selected from the group consisting a pluripotent stem cell, an induced pluripotent stem cell, a T cell differentiated from an induced pluripotent stem cell, a primary T cell, and a cell derived from a primary T cell, and the hypoimmunogenic cell comprises exogenous CD47 and/or optionally a CAR, and wherein the hypoimmunogenic cell is optionally a B2M^{indel/indel} cell and/or optionally CIITA^{indel/indel} cell.

176. A population of hypoimmunogenic cells comprising exogenous CD47 polypeptides and reduced expression of MHC class I and/or class II human leukocyte antigens, wherein the population of hypoimmunogenic cells comprises cells derived from primary T cells.

177. A population of hypoimmunogenic cells comprising exogenous CD47 polypeptides and reduced expression of MHC class I and class II human leukocyte antigens, wherein the population of hypoimmunogenic cells comprises cells derived from primary T cells.

178. A population of hypoimmunogenic cells comprising exogenous CD47 polypeptides and reduced levels of B2M and CIITA polypeptides, wherein the population of hypoimmunogenic cells comprises cells derived from primary T cells.

179. A population of hypoimmunogenic cells comprising exogenous CD47 polypeptides, a genomic modification of the B2M gene, and a genomic modification of the CIITA gene, wherein the population of hypoimmunogenic cells comprises cells derived from primary T cells.

180. The population of hypoimmunogenic cells of any one of claims 176-179, wherein the cells derived from primary T cells are derived from a pool of T cells comprising primary T cells from one or more, optionally, two or more, three or more, four or more, five or more, ten or more, twenty or more, fifty or more, or one hundred or more, subjects different from the patient.

181. The population of hypoimmunogenic cells of any one of claims 176-180, wherein the cells derived from primary T cells comprise a chimeric antigen receptor (CAR).

182. The population of hypoimmunogenic cells of any one of claims 176-181, wherein the cells derived from primary T cells comprise reduced expression of an endogenous T cell receptor.

183. The population of hypoimmunogenic cells of any one of claims 176-182, wherein the cells derived from primary T cells comprise reduced expression of cytotoxic T-lymphocyte-associated protein 4 (CTLA4) and/or programmed cell death (PD1).

184. The population of hypoimmunogenic cells of any one of claims 181-183, wherein the chimeric antigen receptor (CAR) is selected from the group consisting of:
(a) a first generation CAR comprising an antigen binding domain, a transmembrane domain, and a signaling domain;
(b) a second generation CAR comprising an antigen binding domain, a transmembrane domain, and at least two signaling domains;
(c) a third generation CAR comprising an antigen binding domain, a transmembrane domain, and at least three signaling domains; and
(d) a fourth generation CAR comprising an antigen binding domain, a transmembrane domain, three or four signaling domains, and a domain which upon successful signaling of the CAR induces expression of a cytokine gene.

185. The population of hypoimmunogenic cells of claim 184, wherein the antigen binding domain is selected from the group consisting of:
(a) an antigen binding domain targets an antigen characteristic of a neoplastic cell;
(b) an antigen binding domain that targets an antigen characteristic of a T cell,
(c) an antigen binding domain targets an antigen characteristic of an autoimmune or inflammatory disorder;
(d) an antigen binding domain that targets an antigen characteristic of senescent cells;
(e) an antigen binding domain that targets an antigen characteristic of an infectious disease; and
(f) an antigen binding domain that binds to a cell surface antigen of a cell.

186. The population of hypoimmunogenic cells of claim 185, wherein the antigen binding domain is selected from the group consisting of an antibody, an antigen-binding portion thereof, an scFv, and a Fab.

187. The population of hypoimmunogenic cells of claim 185 or 186, wherein the antigen binding domain binds to CD19, CD20, CD22, or BCMA.

188. The population of hypoimmunogenic cells of any one of claims 184-187, wherein the transmembrane domain comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

189. The population of hypoimmunogenic cells of any one of claims 184-188, wherein the signaling domain(s) comprises a costimulatory domain(s).

190. The population of hypoimmunogenic cells of claim 189, wherein the costimulatory domains comprise two costimulatory domains that are not the same.

191. The population of hypoimmunogenic cells of claim 189 or 190, wherein the costimulatory domain(s) enhances cytokine production, CAR T cell proliferation, and/or CAR T cell persistence during T cell activation.

192. The population of hypoimmunogenic cells of any one of claims 184-191, wherein the cytokine gene is an endogenous or exogenous cytokine gene to the hypoimmunogenic cells.

193. The population of hypoimmunogenic cells of claim 192, wherein the cytokine gene encodes a pro-inflammatory cytokine.

194. The population of hypoimmunogenic cells of claim 193, wherein the pro-inflammatory cytokine is selected from the group consisting of IL-1, IL-2, IL-9, IL-12, IL-18, TNF, IFN-gamma, and a functional fragment thereof.

195. The population of hypoimmunogenic cells of any one of claims 184-194, wherein the domain which upon successful signaling of the CAR induces expression of the cytokine gene comprises a transcription factor or functional domain or fragment thereof.

196. The population of hypoimmunogenic cells of any one of claims 181-195, wherein the CAR comprises a CD3 zeta domain or an immunoreceptor tyrosine-based activation motif (ITAM), or functional variant thereof.

197. The population of hypoimmunogenic cells of any one of claims 181-196, wherein the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

198. The population of hypoimmunogenic cells of any one of claims 181-197, wherein the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

199. The population of hypoimmunogenic cells of any one of claims 181-198, wherein the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

200. The population of hypoimmunogenic cells of any one of claims 181-197, wherein the CAR comprises a (i) an anti-CD19 scFv; (ii) a CD8α hinge and transmembrane domain or functional variant thereof; (iii) a 4-1BB costimulatory domain or functional variant thereof; and (iv) a CD3ζ signaling domain.

201. The population of hypoimmunogenic cells of any one of claims 176-200, for use in treatment of a disorder in a patient, wherein the patient has previously received an initial population of such hypoimmunogenic cells.

202. The population of hypoimmunogenic cells of any one of claims 176-201, wherein the hypoimmunogenic cell is selected from the group consisting a pluripotent stem cell, an induced pluripotent stem cell, a T cell differentiated from an induced pluripotent stem cell, a primary T cell, and a cell derived from a primary T cell, and the hypoimmunogenic cell comprises exogenous CD47 and/or optionally a CAR, and wherein the hypoimmunogenic cell is optionally a B2M^{indel/indel} cell and/or optionally CIITA^{indel/indel} cell.

203. Use of a population of hypoimmunogenic cells comprising exogenous CD47 polypeptides and reduced expression of MHC class I and/or class II human leukocyte antigens, wherein the population of hypoimmunogenic cells comprises cells derived from primary T cells.

204. Use of a population of hypoimmunogenic cells comprising exogenous CD47 polypeptides and reduced expression of MHC class I and class II human leukocyte antigens, wherein the population of hypoimmunogenic cells comprises cells derived from primary T cells.

205. Use of a population of hypoimmunogenic cells comprising exogenous CD47 polypeptides and reduced levels of B2M and CIITA polypeptides, wherein the population of hypoimmunogenic cells comprises cells derived from primary T cells.

206. Use of a population of hypoimmunogenic cells comprising exogenous CD47 polypeptides, a genomic modification of the B2M gene, and a genomic modification of the CIITA gene, wherein the population of hypoimmunogenic cells comprises cells derived from primary T cells.

207. The use of the population of hypoimmunogenic cells of any one of claims 203-206, wherein the cells derived from primary T cells are derived from a pool of T cells comprising T cells from one or more subjects different from the patient.

208. The use of the population of hypoimmunogenic cells of any one of claims use of the 203-207, wherein the cells derived from primary T cells comprise a chimeric antigen receptor (CAR).

209. The use of the population of hypoimmunogenic cells of any one of claims 203-208, wherein the cells derived from primary T cells comprise reduced expression of an endogenous T cell receptor.

210. The use of the population of hypoimmunogenic cells of any one of claims 203-209, wherein the cells derived from primary T cells comprise reduced expression of cytotoxic T-lymphocyte-associated protein 4 (CTLA4) and/or programmed cell death (PD1).

211. The use of the population of hypoimmunogenic cells of any one of claims 208-210, wherein the chimeric antigen receptor (CAR) is selected from the group consisting of:
(a) a first generation CAR comprising an antigen binding domain, a transmembrane domain, and a signaling domain;
(b) a second generation CAR comprising an antigen binding domain, a transmembrane domain, and at least two signaling domains;
(c) a third generation CAR comprising an antigen binding domain, a transmembrane domain, and at least three signaling domains; and
(d) a fourth generation CAR comprising an antigen binding domain, a transmembrane domain, three or four signaling domains, and a domain which upon successful signaling of the CAR induces expression of a cytokine gene.

212. The use of the population of hypoimmunogenic cells of claim 211, wherein the antigen binding domain is selected from the group consisting of:
(a) an antigen binding domain targets an antigen characteristic of a neoplastic cell;
(b) an antigen binding domain that targets an antigen characteristic of a T cell,
(c) an antigen binding domain targets an antigen characteristic of an autoimmune or inflammatory disorder;
(d) an antigen binding domain that targets an antigen characteristic of senescent cells;
(e) an antigen binding domain that targets an antigen characteristic of an infectious disease; and
(f) an antigen binding domain that binds to a cell surface antigen of a cell.

213. The use of the population of hypoimmunogenic cells of claim 212, wherein the antigen binding domain is selected from the group consisting of an antibody, an antigen-binding portion thereof, an scFv, and a Fab.

214. The use of the population of hypoimmunogenic cells of claim 212 or 213, wherein the antigen binding domain binds to CD19, CD20, CD22, or BCMA.

215. The use of the population of hypoimmunogenic cells of any one of claims 211-214, wherein the transmembrane domain comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

216. The use of the population of hypoimmunogenic cells of any one of claims 211-215, wherein the signaling domain(s) comprises a costimulatory domain(s).

217. The use of the population of hypoimmunogenic cells of claim 216, wherein the costimulatory domains comprise two costimulatory domains that are not the same.

218. The use of the population of hypoimmunogenic cells of claim 216 or 217, wherein the costimulatory domain(s) enhances cytokine production, CAR T cell proliferation, and/or CAR T cell persistence during T cell activation.

219. The use of the population of hypoimmunogenic cells of any one of claims 211-218, wherein the cytokine gene is an endogenous or exogenous cytokine gene to the hypoimmunogenic cells.

220. The use of the population of hypoimmunogenic cells of claim 219, wherein the cytokine gene encodes a pro-inflammatory cytokine.

221. The use of the population of hypoimmunogenic cells of claim 220, wherein the pro-inflammatory cytokine is selected from the group consisting of IL-1, IL-2, IL-9, IL-12, IL-18, TNF, IFN-gamma, and a functional fragment thereof.

222. The use of the population of hypoimmunogenic cells of any one of claims 211-221, wherein the domain which upon successful signaling of the CAR induces expression of the cytokine gene comprises a transcription factor or functional domain or fragment thereof.

223. The use of the population of hypoimmunogenic cells of any one of claims 208-222, wherein the CAR comprises a CD3 zeta domain or an immunoreceptor tyrosine-based activation motif (ITAM), or functional variant thereof.

224. The use of the population of hypoimmunogenic cells of any one of claims 208-223, wherein the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

225. The use of the population of hypoimmunogenic cells of any one of claims 208-224, wherein the CAR comprises a (i) a CD3 zeta domain, or an immunoreceptor tyrosine-based activation motif (ITAM), or functional variant thereof; (ii) a CD28 domain or functional variant thereof; and (iii) a 4-1BB costimulatory domain, or a CD134 domain, or functional variant thereof.

226. The use of the population of hypoimmunogenic cells of any one of claims 208-225, wherein the CAR comprises a (i) a CD3 zeta domain, or an immunoreceptor tyrosine-based activation motif (ITAM), or functional variant thereof; (ii) a CD28 domain or functional variant thereof; (iii) a 4-1BB costimulatory domain, or a CD134 domain, or functional variant thereof; and (iv) a cytokine or costimulatory ligand transgene.

227. The use of the population of hypoimmunogenic cells of any one of claims 203-224, wherein the CAR comprises a (i) an anti-CD19 scFv; (ii) a CD8α hinge and transmembrane domain or functional variant thereof; (iii) a 4-1BB costimulatory domain or functional variant thereof; and (iv) a CD3ζ signaling domain.

228. The use of the population of hypoimmunogenic cells of any one of claims 203-227, for use in treatment of a disorder in a patient, wherein the patient has previously received an initial population of such hypoimmunogenic cells.

229. The use of the population of hypoimmunogenic cells of any one of claims 176-228, wherein the hypoimmunogenic cell is selected from the group consisting a pluripotent stem cell, an induced pluripotent stem cell, a T cell differentiated from an induced pluripotent stem cell, a primary T cell, and a cell derived from a primary T cell, and the hypoimmunogenic cell comprises exogenous CD47 and/or optionally a CAR, and wherein the hypoimmunogenic cell is optionally a B2M^{indel/indel} cell and/or optionally CIITA^{indel/indel} cell.

230. A method for treating a disorder in a patient comprising administering to the patient a therapeutically effective amount of a population of hypoimmunogenic T cells derived from primary T cells and which comprise exogenous CD47 polypeptides and exhibit reduced expression of MHC class I and/or class II human leukocyte antigens, wherein an initial population of such hypoimmunogenic T cells had previously been administered to the patient.

231. The method of claim 230, wherein the hypoimmunogenic T cells comprise reduced expression of MHC class I and class II human leukocyte antigens.

232. The method of claim 230 or 231, wherein the hypoimmunogenic T cells express the exogenous CD47 polypeptides and reduced expression levels of B2M and/or CIITA.

233. The method of any one of claims 230-232, wherein the hypoimmunogenic T cells express the exogenous CD47 polypeptides and reduced expression levels of B2M and CIITA.

234. The method of claim 230-233, wherein the hypoimmunogenic T cells comprise a chimeric antigen receptor.

235. The method of claim 234, wherein the chimeric antigen receptor (CAR) is selected from the group consisting of:
(a) a first generation CAR comprising an antigen binding domain, a transmembrane domain, and a signaling domain;
(b) a second generation CAR comprising an antigen binding domain, a transmembrane domain, and at least two signaling domains;
(c) a third generation CAR comprising an antigen binding domain, a transmembrane domain, and at least three signaling domains; and
(d) a fourth generation CAR comprising an antigen binding domain, a transmembrane domain, three or four signaling domains, and a domain which upon successful signaling of the CAR induces expression of a cytokine gene.

236. The method of claim 235, wherein the antigen binding domain is selected from the group consisting of:
(a) an antigen binding domain targets an antigen characteristic of a neoplastic cell;
(b) an antigen binding domain that targets an antigen characteristic of a T cell;
(c) an antigen binding domain targets an antigen characteristic of an autoimmune or inflammatory disorder;
(d) an antigen binding domain that targets an antigen characteristic of senescent cells;
(e) an antigen binding domain that targets an antigen characteristic of an infectious disease; and
(f) an antigen binding domain that binds to a cell surface antigen of a cell.

237. The method of claim 234 or 235, wherein the antigen binding domain is selected from the group consisting of an antibody, an antigen-binding portion thereof, an scFv, and a Fab.

238. The method of any one of claims 234-237, wherein the antigen binding domain binds to CD19, CD20, CD22, or BCMA.

239. The method of any one of claims 234-238, wherein the transmembrane domain comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

240. The method of any one of claims 234-239, wherein the signaling domain(s) comprises a costimulatory domain(s).

241. The method of claim 240, wherein the costimulatory domains comprise two costimulatory domains that are not the same.

242. The method of claim 240 or 241, wherein the costimulatory domain(s) enhances cytokine production, CAR T cell proliferation, and/or CAR T cell persistence during T cell activation.

243. The method of any one of claims 235-242, wherein the cytokine gene is an endogenous or exogenous cytokine gene to the hypoimmunogenic T cells.

244. The method of claim 243, wherein the cytokine gene encodes a pro-inflammatory cytokine.

245. The method of claim 244, wherein the pro-inflammatory cytokine is selected from the group consisting of IL-1, IL-2, IL-9, IL-12, IL-18, TNF, IFN-gamma, and a functional fragment thereof.

246. The method of any one of claims 233-245, wherein the domain which upon successful signaling of the CAR induces expression of the cytokine gene comprises a transcription factor or functional domain or fragment thereof.

247. The method of any one of claims 233-246, wherein the CAR comprises a CD3 zeta domain or an immunoreceptor tyrosine-based activation motif (ITAM), or functional variant thereof.

248. The method of any one of claims 233-247, wherein the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

249. The method of any one of claims 233-248, wherein the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

250. The method of any one of claims 233-249, wherein the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

251. The method of any one of claims 233-250, wherein the CAR comprises a (i) an anti-CD19 scFv; (ii) a CD8α hinge and transmembrane domain or functional variant thereof; (iii) a 4-1BB costimulatory domain or functional variant thereof; and (iv) a CD3ζ signaling domain or functional variant thereof.

252. The method of any one of claims 230-251, wherein the hypoimmunogenic T cells comprise reduced expression of an endogenous T cell receptor.

253. The method of any one of claims 230-252, wherein the hypoimmunogenic T cells comprise reduced expression of cytotoxic T-lymphocyte-associated protein 4 (CTLA4) and/or programmed cell death (PD1).

254. The method of any one of claims 230-253, wherein the population of the hypoimmunogenic T cells is administered at least 3 days or more after the initial administration, optionally at least 4 days, 5 days, 6 days, 7 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 1 month, 2 months, 3 months, 4 months, 5 months, 6 months, 12 months, 18 months, 24 months, 30 months, 36 months, 48 months, 54 months, or 60 months or more.

255. The method of any one of claims 230-254, wherein the population of the hypoimmunogenic T cells is administered at least 3 days to at least 7 days or more after the initial administration.

256. The method of any one of claims 230-255, wherein the population of the hypoimmunogenic T cells is administered least 1 month or more after the initial administration.

257. The method of any one of claims 230-256, wherein the population of the hypoimmunogenic T cells is administered at least 2 months or more after the initial administration.

258. The method of any one of claims 230-257, wherein upon the initial and/or subsequent administrations, the population of hypoimmunogenic T cells elicits a reduced level of immune activation or no immune activation in the patient.

259. The method of any one of claims 230-258, wherein upon the initial and/or subsequent administrations, the population of hypoimmunogenic T cells elicits a reduced level of systemic TH1 activation or no systemic TH1 activation in the patient.

260. The method of any one of claims 230-259, wherein upon the initial and/or subsequent administrations, the population of hypoimmunogenic T cells elicits a reduced level of immune activation of peripheral blood mononuclear cells (PBMCs) or no immune activation of PBMCs in the patient.

261. The method of any one of claims 230-260, wherein upon the initial and/or subsequent administrations, the population of hypoimmunogenic T cells elicits a reduced level of donor-specific IgG antibodies or no donor specific IgG antibodies against the hypoimmunogenic cells in the patient.

262. The method of any one of claims 230-261, wherein upon the initial and/or subsequent administrations, the population of hypoimmunogenic T cells elicits a reduced level of IgM and IgG antibody production or no IgM and IgG antibody production against the hypoimmunogenic cells in the patient.

263. The method of any one of claims 230-262, wherein upon administration, the population of hypoimmunogenic T cells elicits a reduced level of cytotoxic T cell killing or no cytotoxic T cell killing of the hypoimmunogenic T cells in the patient.

264. The method of any one of claims 230-263, wherein the population of hypoimmunogenic T cells of the initial administration are no longer present in the patient at the subsequent administration.

265. The method of any one of claims 230-264, wherein the patient is not administered an immunosuppressive agent at least 3 days or more before or after the initial administration of the population of hypoimmunogenic T cells.

266. The method of any one of claims 230-265, wherein the patient is not administered an immunosuppressive agent at least 3 days or more before or after the initial subsequent administration of the population of hypoimmunogenic T cells.

267. The method of any one of claims 230-266, wherein the hypoimmunogenic cell is a B2M^{indel/indel}, CIITA^{indel/indel} cell comprising the exogenous CD47 polypeptides and optionally a CAR.
